SEQUENCE LISTING (169 sequences)

SEQ ID NO: 1 - protein, 641 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMDLTAIYESLLSLSPDVPVPSDHGGTESSPGWGSSGPWSLSPSDSSPSGVTSRLPGRSTSLVEGRSCGWVPPPPGFAPLAPRLGPELSPSPTSPTATSTTPSRYKTELCRTFSESGRCRYGAKCQFAHGLGELRQANRHPKYKTELCHKFYLQGRCPYGSRCHFIHNPSEDLAAPGHPPVLRQSISFSGLPSGRRTSPPPPGLAGPSLSSSSFSPSSSPPPPGDLPLSPSAFSAAPGTPLARRDPTPVCCPSCRRATPISVWGPLGGLVRTPSVQSLGSDPDEYASSGSSLGGSDSPVFEAGVFAPPQPVAAPRRLPIFNRISVSEGGSGGSGGSGGSGGSGGSDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 2 - protein, 600 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMPAATVDHSQRICEVWACNLDEEMKKIRQVIRKYNYVAMDTEFPGVVARPIGEFRSNADYQYQLLRCNVDLLKIIQLGLTFMNEQGEYPPGTSTWQFNFKFNLTEDMYAQDSIELLTTSGIQFKKHEEEGIETQYFAELLMTSGVVLCEGVKWLSFHSGYDFGYLIKILTNSNLPEEELDFFEILRLFFPVIYDVKYLMKSCKNLKGGLQEVAEQLELERIGPQHQAGSDSLLTGMAFFKMREMFFEDHIDDAKYCGHLYGLGSGSSYVQNGTGNAYEEEANKQSGGSGGSGGSGGSGGSGGSDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 3 - protein, 836 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMPAATVDHSQRICEVWACNLDEEMKKIRQVIRKYNYVAMDTEFPGVVARPIGEFRSNADYQYQLLRCNVDLLKIIQLGLTFMNEQGEYPPGTSTWQFNFKFNLTEDMYAQDSIELLTTSGIQFKKHEEEGIETQYFAELLMTSGVVLCEGVKWLSFHSGYDFGYLIKILTNSNLPEEELDFFEILRLFFPVIYDVKYLMKSCKNLKGGLQEVAEQLELERIGPQHQAGSDSLLTGMAFFKMREMFFEDHIDDAKYCGHLYGLGSGSSYVQNGTGNAYEEEANKQSGGSGGSGGSGGSGGSGGSMDLTAIYESLLSLSPDVPVPSDHGGTESSPGWGSSGPWSLSPSDSSPSGVTSRLPGRSTSLVEGRSCGWVPPPPGFAPLAPRLGPELSPSPTSPTATSTTPSRYKTELCRTFSESGRCRYGAKCQFAHGLGELRQANRHPKYKTELCHKFYLQGRCPYGSRCHFIHNPSEDLAAPGHPPVLRQSISFSGLPSGRRTSPPPPGLAGPSLSSSSFSPSSSPPPPGDLPLSPSAFSAAPGTPLARRDPTPVCCPSCRRATPISVWGPLGGLVRTPSVQSLGSDPDEYASSGSSLGGSDSPVFEAGVFAPPQPVAAPRRLPIFNRISVSEGSDYKDDDDK

SEQ ID NO: 4 - protein, 627 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSTRSRYASDFEEIAVLGQGAFGQVVKARNALDSRYYAIKKIRHTEEKLSTILSEVMLLASLNHQYVVRYYAAWLEEDSMDENVFESTDEESDLSESSSDFEENDLLDQSSIFKNRTNHDLDNSNWDFISGSGYPDIVFENSSRDDENEDLDHDTSSTSSSESQDDTDKESKSIQNVPRRRNFVKPMTAVKKKSTLFIQMEYCENGTLYDLIHSENLNQQRDEYWRLFRQILEALSYIHSQGIIHRDLKPMNILIDESRNVKIGDFGLAKNVHRSLDILKLDSQNLPGSSDNLTSAIGTAMYVATEVLDGTGHYNEKIDMYSLGIIFFEMIYPFSTGMERVNILKKLRSVSIEFPPDFDDNKMKVEKKIIRLLIDHDPNKRPGARTLLNSGWLPVKHQDEVIKEALKSLSNPSSPWQQQVREGSDYKDDDDK

SEQ ID NO: 5 - protein, 324 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSGGSSCSQTPSRAIPTRRVALGDGVQLPPGDYSTAPGGTLFSTAPGGTRIIYDRKFLMECRNAPVAKAPPKDLPTIPGVTSPTSDEPPMQASQSHLHSSPEDKRAGGEESQFEMDIGSDYKDDDDK

SEQ ID NO: 6 - protein, 399 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMGTFDLWTDYLGLAHLVRALSGKEGPETRLSPQPEPEPMLEPVSALEPMPAPESVPVPGPKDQKRSLESSPAPERLCSFCKHNGESRAIYQSHVLKDEAGRVLCPILRDYVCPQCGATRERAHTRRFCPLTGQGYTSVYSHTTRNSAGKKLVRPDKAKTQDTGHRRGGGGGAGFRGAGKSEPSPSCSPSMSTGSDYKDDDDK

SEQ ID NO: 7 - protein, 848 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMATVEPETTPTPNPPTTEEEKTESNQEVANPEHYIKHPLQNRWALWFFKNDKSKTWQANLRLISKFDTVEDFWALYNHIQLSSNLMPGCDYSLFKDGIEPMWEDEKNKRGGRWLITLNKQQRRSDLDRFWLETLLCLIGESFDDYSDDVCGAVVNVRAKGDKIAIWTTECENREAVTHIGRVYKERLGLPPKIVIGYQSHADTATKSGSTTKNRFVVGGSGGSGGSGGSGGSGGSMSASQDSRSRDNGPDGMEPEGVIESNWNEIVDSFDDMNLSESLLRGIYAYGFEKPSAIQQRAILPCIKGYDVIAQAQSGTGKTATFAISILQQIELDLKATQALVLAPTRELAQQIQKVVMALGDYMGASCHACIGGTNVRAEVQKLQMEAPHIIVGTPGRVFDMLNRRYLSPKYIKMFVLDEADEMLSRGFKDQIYDIFQKLNSNTQVVLLSATMPSDVLEVTKKFMRDPIRILVKKEELTLEGIRQFYINVEREEWKLDTLCDLYETLTITQAVIFINTRRKVDWLTEKMHARDFTVSAMHGDMDQKERDVIMREFRSGSSRVLITTDLLARGIDVQQVSLVINYDLPTNRENYIHRIGRGGRFGRKGVAINMVTEEDKRTLRDIETFYNTSIEEMPLNVADLIGSDYKDDDDK

SEQ ID NO: 8 - protein, 1110 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSASQDSRSRDNGPDGMEPEGVIESNWNEIVDSFDDMNLSESLLRGIYAYGFEKPSAIQQRAILPCIKGYDVIAQAQSGTGKTATFAISILQQIELDLKATQALVLAPTRELAQQIQKVVMALGDYMGASCHACIGGTNVRAEVQKLQMEAPHIIVGTPGRVFDMLNRRYLSPKYIKMFVLDEADEMLSRGFKDQIYDIFQKLNSNTQVVLLSATMPSDVLEVTKKFMRDPIRILVKKEELTLEGIRQFYINVEREEWKLDTLCDLYETLTITQAVIFINTRRKVDWLTEKMHARDFTVSAMHGDMDQKERDVIMREFRSGSSRVLITTDLLARGIDVQQVSLVINYDLPTNRENYIHRIGRGGRFGRKGVAINMVTEEDKRTLRDIETFYNTSIEEMPLNVADLIGGSGGSGGSGGSGGSGGSMSDGFDRAPGAGRGRSRGLGRGGGGPEGGGFPNGAGPAERARHQPPQPKAPGFLQPPPLRQPRTTPPPGAQCEVPASPQRPSRPGALPEQTRPLRAPPSSQDKIPQQNSESAMAKPQVVVAPVLMSKLSVNAPEFYPSGYSSSYTESYEDGCEDYPTLSEYVQDFLNHLTEQPGSFETEIEQFAETLNGCVTTDDALQELVELIYQQATSIPNFSYMGARLCNYLSHHLTISPQSGNFRQLLLQRCRTEYEVKDQAAKGDEVTRKRFHAFVLFLGELYLNLEIKGTNGQVTRADILQVGLRELLNALFSNPMDDNLICAVKLLKLTGSVLEDAWKEKGKMDMEEIIQRIENVVLDANCSRDVKQMLLKLVELRSSNWGRVHATSTYREATPENDPNYFMNEPTFYTSDGVPFTAADPDYQEKYQELLEREDFFPDYEENGTDLSGAGDPYLDDIDDEMDPEIEEAYEKFCLESERKRKQGSDYKDDDDK

SEQ ID NO: 9 - protein, 1004 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSATSVDTQRTKGQDNKVQNGSLHQKDTVHDNDFEPYLTGQSNQSNSYPSMSDPYLSSYYPPSIGFPYSLNEAPWSTAGDPPIPYLTTYGQLSNGDHHFMHDAVFGQPGGLGNNIYQHRFNFFPENPAFSAWGTSGSQGQQTQSSAYGSSYTYPPSSLGGTVVDGQPGFHSDTLSKAPGMNSLEQGMVGLKIGDVSSSAVKTVGSVVSSVALTGVLSGNGGTNVNMPVSKPTSWAAIASKPAKPQPKMKTKSGPVMGGGLPPPPIKHNMDIGTWDNKGPVPKAPVPQQAPSPQAAPQPQQVAQPLPAQPPALAQPQYQSPQQPPQTRWVAPRNRNAAFGQSGGAGSDSNSPGNVQPNSAPSVESGGSGGSGGSGGSGGSGGSMSATSVDQRPKGQGNKVSVQNGSIHQKDAVNDDDFEPYLSSQTNQSNSYPPMSDPYMPSYYAPSIGFPYSLGEAAWSTAGDQPMPYLTTYGQMSNGEHHYIPDGVFSQPGALGNTPPFLGQHGFNFFPGNADFSTWGTSGSQGQSTQSSAYSSSYGYPPSSLGRAITDGQAGFGNDTLSKVPGISSIEQGMTGLKIGGDLTAAVTKTVGTALSSSGMTSIATNSVPPVSSAAPKPTSWAAIARKPAKPQPKLKPKGNVGIGGSAVPPPPIKHNMNIGTWDEKGSVVKAPPTQPVLPPQTIIQQPQPLIQPPPLVQSQLPQQQPQPPQPQQQQGPQPQAQPHQVQPQQQQLQNRWVAPRNRGAGFNQNNGAGSENFGLGVVPVSASPSSVEVHPVLEKLKAINNYNPKDFDWNLKNGSDYKDDDDK

SEQ ID NO: 10 - protein, 540 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSGDEMIFDPTMSKKKKKKKKPFMLDEEGDTQTEETQPSETKEVEPEPTEDKDLEADEEDTRKKDASDDLDDLNFFNQKKKKKKTKKIFDIDEAEEGVKDLKIESDVQEPTEPEDDLDIMLGNKKKKKKNVKFPDEDEILEKDEALEDEDNKKDDGISFSNQTGPAWAGSERDYTYEELLNRVFNIMREKNPDMVAGEKRKFVMKPPQVVRVGTKKTSFVNFTDICKLLHRQPKHLLAFLLAELGTSGSIDGNNQLVIKGRFQQKQIENVLRRYIKEYVTCHTCRSPDTILQKDTRLYFLQCETCHSRCSVASIKTGFQAVTGKRAQLRAKANGSDYKDDDDK

SEQ ID NO: 11 - protein, 413 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAKLGSDYKDDDDK

SEQ ID NO: 12 - protein, 462 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMDDREDLVYQAKLAEQAERYDEMVESMKKVAGMDVELTVEERNLLSVAYKNVIGARRASWRIISSIEQKEENKGGEDKLKMIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWTSDMQGDGEEQNKEALQDVEDENQGSDYKDDDDK

SEQ ID NO: 13 - protein, 606 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMAEEMVEAAGEDERELAAEMAAAFLNENLPESIFGAPKAGNGQWASVIRVMNPIQGNTLDLVQLEQNEAAFSVAVCRFSNIGEDWYVLVGVAKDLILNPRSVAGGFVYTYKLVNNGEKLEFLHKTPVEEVPAAIAPFQGRVLIGVGKLLRVYDLGKKKLLRKCENKHIANYISGIQTIGHRVIVSDVQESFIWVRYKRNENQLIIFADDTYPRWVTTASLLDYDTVAGADKFGNICVVRLPPNTNDEVDEDPTGNKALWDRGLLNGASQKAEVIMNYHVGETVLSLQKTTLIPGGSESLVYTTLSGGIGILVPFTSHEDHDFFQHVEMHLRSEHPPLCGRDHLSFRSYYFPVKNVIDGDLCEQFNSMEPNKQKNVSEELDRTPPEVSKKLEDIRTRYAFGSDYKDDDDK

SEQ ID NO: 14 - protein, 46 aa, synthetic construct
MGIINTLQKYYCRVRGGRCAVLSCLPKEEQIGKCSTRGRKCCRRKK

SEQ ID NO: 15 - protein, 18 aa, synthetic construct
GGSGGSGGSGGSGGSGGS

SEQ ID NO: 16 - protein, 102 aa, synthetic construct
MAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFV

SEQ ID NO: 17 - protein, 11 aa, synthetic construct
LQLPPLERLTL

SEQ ID NO: 18 - protein, 8 aa, synthetic construct
DYKDDDDK

SEQ ID NO: 19 - protein, 434 aa, synthetic construct
MDLTAIYESLLSLSPDVPVPSDHGGTESSPGWGSSGPWSLSPSDSSPSGVTSRLPGRSTSLVEGRSCGWVPPPPGFAPLAPRLGPELSPSPTSPTATSTTPSRYKTELCRTFSESGRCRYGAKCQFAHGLGELRQANRHPKYKTELCHKFYLQGRCPYGSRCHFIHNPSEDLAAPGHPPVLRQSISFSGLPSGRRTSPPPPGLAGPSLSSSSFSPSSSPPPPGDLPLSPSAFSAAPGTPLARRDPTPVCCPSCRRATPISVWGPLGGLVRTPSVQSLGSDPDEYASSGSSLGGSDSPVFEAGVFAPPQPVAAPRRLPIFNRISVSEGGSGGSGGSGGSGGSGGSDAMFGQPGALGSTPFLGQHGFNFFPSGIDFSAWGNNSSQGQSTQSSGYSSNYAYAPSSLGGAMIDGQSAFANETLNKAPGMNTIDQGMAA

SEQ ID NO: 20 - protein, 393 aa, synthetic construct
MPAATVDHSQRICEVWACNLDEEMKKIRQVIRKYNYVAMDTEFPGVVARPIGEFRSNADYQYQLLRCNVDLLKIIQLGLTFMNEQGEYPPGTSTWQFNFKFNLTEDMYAQDSIELLTTSGIQFKKHEEEGIETQYFAELLMTSGVVLCEGVKWLSFHSGYDFGYLIKILTNSNLPEEELDFFEILRLFFPVIYDVKYLMKSCKNLKGGLQEVAEQLELERIGPQHQAGSDSLLTGMAFFKMREMFFEDHIDDAKYCGHLYGLGSGSSYVQNGTGNAYEEEANKQSGGSGGSGGSGGSGGSGGSDAMFGQPGALGSTPFLGQHGFNFFPSGIDFSAWGNNSSQGQSTQSSGYSSNYAYAPSSLGGAMIDGQSAFANETLNKAPGMNTIDQGMAA

SEQ ID NO: 21 - protein, 629 aa, synthetic construct
MPAATVDHSQRICEVWACNLDEEMKKIRQVIRKYNYVAMDTEFPGVVARPIGEFRSNADYQYQLLRCNVDLLKIIQLGLTFMNEQGEYPPGTSTWQFNFKFNLTEDMYAQDSIELLTTSGIQFKKHEEEGIETQYFAELLMTSGVVLCEGVKWLSFHSGYDFGYLIKILTNSNLPEEELDFFEILRLFFPVIYDVKYLMKSCKNLKGGLQEVAEQLELERIGPQHQAGSDSLLTGMAFFKMREMFFEDHIDDAKYCGHLYGLGSGSSYVQNGTGNAYEEEANKQSGGSGGSGGSGGSGGSGGSMDLTAIYESLLSLSPDVPVPSDHGGTESSPGWGSSGPWSLSPSDSSPSGVTSRLPGRSTSLVEGRSCGWVPPPPGFAPLAPRLGPELSPSPTSPTATSTTPSRYKTELCRTFSESGRCRYGAKCQFAHGLGELRQANRHPKYKTELCHKFYLQGRCPYGSRCHFIHNPSEDLAAPGHPPVLRQSISFSGLPSGRRTSPPPPGLAGPSLSSSSFSPSSSPPPPGDLPLSPSAFSAAPGTPLARRDPTPVCCPSCRRATPISVWGPLGGLVRTPSVQSLGSDPDEYASSGSSLGGSDSPVFEAGVFAPPQPVAAPRRLPIFNRISVSE

SEQ ID NO: 22 - protein, 420 aa, synthetic construct
TRSRYASDFEEIAVLGQGAFGQVVKARNALDSRYYAIKKIRHTEEKLSTILSEVMLLASLNHQYVVRYYAAWLEEDSMDENVFESTDEESDLSESSSDFEENDLLDQSSIFKNRTNHDLDNSNWDFISGSGYPDIVFENSSRDDENEDLDHDTSSTSSSESQDDTDKESKSIQNVPRRRNFVKPMTAVKKKSTLFIQMEYCENGTLYDLIHSENLNQQRDEYWRLFRQILEALSYIHSQGIIHRDLKPMNILIDESRNVKIGDFGLAKNVHRSLDILKLDSQNLPGSSDNLTSAIGTAMYVATEVLDGTGHYNEKIDMYSLGIIFFEMIYPFSTGMERVNILKKLRSVSIEFPPDFDDNKMKVEKKIIRLLIDHDPNKRPGARTLLNSGWLPVKHQDEVIKEALKSLSNPSSPWQQQVRE

SEQ ID NO: 23 - protein, 117 aa, synthetic construct
MSGGSSCSQTPSRAIPTRRVALGDGVQLPPGDYSTAPGGTLFSTAPGGTRIIYDRKFLMECRNAPVAKAPPKDLPTIPGVTSPTSDEPPMQASQSHLHSSPEDKRAGGEESQFEMDI

SEQ ID NO: 24 - protein, 192 aa, synthetic construct
MGTFDLWTDYLGLAHLVRALSGKEGPETRLSPQPEPEPMLEPVSALEPMPAPESVPVPGPKDQKRSLESSPAPERLCSFCKHNGESRAIYQSHVLKDEAGRVLCPILRDYVCPQCGATRERAHTRRFCPLTGQGYTSVYSHTTRNSAGKKLVRPDKAKTQDTGHRRGGGGGAGFRGAGKSEPSPSCSPSMST

SEQ ID NO: 25 - protein, 641 aa, synthetic construct
MATVEPETTPTPNPPTTEEEKTESNQEVANPEHYIKHPLQNRWALWFFKNDKSKTWQANLRLISKFDTVEDFWALYNHIQLSSNLMPGCDYSLFKDGIEPMWEDEKNKRGGRWLITLNKQQRRSDLDRFWLETLLCLIGESFDDYSDDVCGAVVNVRAKGDKIAIWTTECENREAVTHIGRVYKERLGLPPKIVIGYQSHADTATKSGSTTKNRFVVGGSGGSGGSGGSGGSGGSMSASQDSRSRDNGPDGMEPEGVIESNWNEIVDSFDDMNLSESLLRGIYAYGFEKPSAIQQRAILPCIKGYDVIAQAQSGTGKTATFAISILQQIELDLKATQALVLAPTRELAQQIQKVVMALGDYMGASCHACIGGTNVRAEVQKLQMEAPHIIVGTPGRVFDMLNRRYLSPKYIKMFVLDEADEMLSRGFKDQIYDIFQKLNSNTQVVLLSATMPSDVLEVTKKFMRDPIRILVKKEELTLEGIRQFYINVEREEWKLDTLCDLYETLTITQAVIFINTRRKVDWLTEKMHARDFTVSAMHGDMDQKERDVIMREFRSGSSRVLITTDLLARGIDVQQVSLVINYDLPTNRENYIHRIGRGGRFGRKGVAINMVTEEDKRTLRDIETFYNTSIEEMPLNVADLI

SEQ ID NO: 26 - protein, 903 aa, synthetic construct
MSASQDSRSRDNGPDGMEPEGVIESNWNEIVDSFDDMNLSESLLRGIYAYGFEKPSAIQQRAILPCIKGYDVIAQAQSGTGKTATFAISILQQIELDLKATQALVLAPTRELAQQIQKVVMALGDYMGASCHACIGGTNVRAEVQKLQMEAPHIIVGTPGRVFDMLNRRYLSPKYIKMFVLDEADEMLSRGFKDQIYDIFQKLNSNTQVVLLSATMPSDVLEVTKKFMRDPIRILVKKEELTLEGIRQFYINVEREEWKLDTLCDLYETLTITQAVIFINTRRKVDWLTEKMHARDFTVSAMHGDMDQKERDVIMREFRSGSSRVLITTDLLARGIDVQQVSLVINYDLPTNRENYIHRIGRGGRFGRKGVAINMVTEEDKRTLRDIETFYNTSIEEMPLNVADLIGGSGGSGGSGGSGGSGGSMSDGFDRAPGAGRGRSRGLGRGGGGPEGGGFPNGAGPAERARHQPPQPKAPGFLQPPPLRQPRTTPPPGAQCEVPASPQRPSRPGALPEQTRPLRAPPSSQDKIPQQNSESAMAKPQVVVAPVLMSKLSVNAPEFYPSGYSSSYTESYEDGCEDYPTLSEYVQDFLNHLTEQPGSFETEIEQFAETLNGCVTTDDALQELVELIYQQATSIPNFSYMGARLCNYLSHHLTISPQSGNFRQLLLQRCRTEYEVKDQAAKGDEVTRKRFHAFVLFLGELYLNLEIKGTNGQVTRADILQVGLRELLNALFSNPMDDNLICAVKLLKLTGSVLEDAWKEKGKMDMEEIIQRIENVVLDANCSRDVKQMLLKLVELRSSNWGRVHATSTYREATPENDPNYFMNEPTFYTSDGVPFTAADPDYQEKYQELLEREDFFPDYEENGTDLSGAGDPYLDDIDDEMDPEIEEAYEKFCLESERKRKQ

SEQ ID NO: 27 - protein, 797 aa, synthetic construct
MSATSVDTQRTKGQDNKVQNGSLHQKDTVHDNDFEPYLTGQSNQSNSYPSMSDPYLSSYYPPSIGFPYSLNEAPWSTAGDPPIPYLTTYGQLSNGDHHFMHDAVFGQPGGLGNNIYQHRFNFFPENPAFSAWGTSGSQGQQTQSSAYGSSYTYPPSSLGGTVVDGQPGFHSDTLSKAPGMNSLEQGMVGLKIGDVSSSAVKTVGSVVSSVALTGVLSGNGGTNVNMPVSKPTSWAAIASKPAKPQPKMKTKSGPVMGGGLPPPPIKHNMDIGTWDNKGPVPKAPVPQQAPSPQAAPQPQQVAQPLPAQPPALAQPQYQSPQQPPQTRWVAPRNRNAAFGQSGGAGSDSNSPGNVQPNSAPSVESGGSGGSGGSGGSGGSGGSMSATSVDQRPKGQGNKVSVQNGSIHQKDAVNDDDFEPYLSSQTNQSNSYPPMSDPYMPSYYAPSIGFPYSLGEAAWSTAGDQPMPYLTTYGQMSNGEHHYIPDGVFSQPGALGNTPPFLGQHGFNFFPGNADFSTWGTSGSQGQSTQSSAYSSSYGYPPSSLGRAITDGQAGFGNDTLSKVPGISSIEQGMTGLKIGGDLTAAVTKTVGTALSSSGMTSIATNSVPPVSSAAPKPTSWAAIARKPAKPQPKLKPKGNVGIGGSAVPPPPIKHNMNIGTWDEKGSVVKAPPTQPVLPPQTIIQQPQPLIQPPPLVQSQLPQQQPQPPQPQQQQGPQPQAQPHQVQPQQQQLQNRWVAPRNRGAGFNQNNGAGSENFGLGVVPVSASPSSVEVHPVLEKLKAINNYNPKDFDWNLKN

SEQ ID NO: 28 - protein, 333 aa, synthetic construct
MSGDEMIFDPTMSKKKKKKKKPFMLDEEGDTQTEETQPSETKEVEPEPTEDKDLEADEEDTRKKDASDDLDDLNFFNQKKKKKKTKKIFDIDEAEEGVKDLKIESDVQEPTEPEDDLDIMLGNKKKKKKNVKFPDEDEILEKDEALEDEDNKKDDGISFSNQTGPAWAGSERDYTYEELLNRVFNIMREKNPDMVAGEKRKFVMKPPQVVRVGTKKTSFVNFTDICKLLHRQPKHLLAFLLAELGTSGSIDGNNQLVIKGRFQQKQIENVLRRYIKEYVTCHTCRSPDTILQKDTRLYFLQCETCHSRCSVASIKTGFQAVTGKRAQLRAKAN

SEQ ID NO: 29 - protein, 206 aa, synthetic construct
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAKL

SEQ ID NO: 30 - protein, 255 aa, synthetic construct
MDDREDLVYQAKLAEQAERYDEMVESMKKVAGMDVELTVEERNLLSVAYKNVIGARRASWRIISSIEQKEENKGGEDKLKMIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWTSDMQGDGEEQNKEALQDVEDENQ

SEQ ID NO: 31 - protein, 399 aa, synthetic construct
MAEEMVEAAGEDERELAAEMAAAFLNENLPESIFGAPKAGNGQWASVIRVMNPIQGNTLDLVQLEQNEAAFSVAVCRFSNIGEDWYVLVGVAKDLILNPRSVAGGFVYTYKLVNNGEKLEFLHKTPVEEVPAAIAPFQGRVLIGVGKLLRVYDLGKKKLLRKCENKHIANYISGIQTIGHRVIVSDVQESFIWVRYKRNENQLIIFADDTYPRWVTTASLLDYDTVAGADKFGNICVVRLPPNTNDEVDEDPTGNKALWDRGLLNGASQKAEVIMNYHVGETVLSLQKTTLIPGGSESLVYTTLSGGIGILVPFTSHEDHDFFQHVEMHLRSEHPPLCGRDHLSFRSYYFPVKNVIDGDLCEQFNSMEPNKQKNVSEELDRTPPEVSKKLEDIRTRYAF

SEQ ID NO: 32 - protein, 326 aa, synthetic construct
MDLTAIYESLLSLSPDVPVPSDHGGTESSPGWGSSGPWSLSPSDSSPSGVTSRLPGRSTSLVEGRSCGWVPPPPGFAPLAPRLGPELSPSPTSPTATSTTPSRYKTELCRTFSESGRCRYGAKCQFAHGLGELRQANRHPKYKTELCHKFYLQGRCPYGSRCHFIHNPSEDLAAPGHPPVLRQSISFSGLPSGRRTSPPPPGLAGPSLSSSSFSPSSSPPPPGDLPLSPSAFSAAPGTPLARRDPTPVCCPSCRRATPISVWGPLGGLVRTPSVQSLGSDPDEYASSGSSLGGSDSPVFEAGVFAPPQPVAAPRRLPIFNRISVSE

SEQ ID NO: 33 - protein, 90 aa, synthetic construct
DAMFGQPGALGSTPFLGQHGFNFFPSGIDFSAWGNNSSQGQSTQSSGYSSNYAYAPSSLGGAMIDGQSAFANETLNKAPGMNTIDQGMAA

SEQ ID NO: 34 - protein, 285 aa, synthetic construct
MPAATVDHSQRICEVWACNLDEEMKKIRQVIRKYNYVAMDTEFPGVVARPIGEFRSNADYQYQLLRCNVDLLKIIQLGLTFMNEQGEYPPGTSTWQFNFKFNLTEDMYAQDSIELLTTSGIQFKKHEEEGIETQYFAELLMTSGVVLCEGVKWLSFHSGYDFGYLIKILTNSNLPEEELDFFEILRLFFPVIYDVKYLMKSCKNLKGGLQEVAEQLELERIGPQHQAGSDSLLTGMAFFKMREMFFEDHIDDAKYCGHLYGLGSGSSYVQNGTGNAYEEEANKQS

SEQ ID NO: 35 - protein, 217 aa, synthetic construct [sequence omitted]

SEQ ID NO: 36 - protein, 406 aa, synthetic construct
MSASQDSRSRDNGPDGMEPEGVIESNWNEIVDSFDDMNLSESLLRGIYAYGFEKPSAIQQRAILPCIKGYDVIAQAQSGTGKTATFAISILQQIELDLKATQALVLAPTRELAQQIQKVVMALGDYMGASCHACIGGTNVRAEVQKLQMEAPHIIVGTPGRVFDMLNRRYLSPKYIKMFVLDEADEMLSRGFKDQIYDIFQKLNSNTQVVLLSATMPSDVLEVTKKFMRDPIRILVKKEELTLEGIRQFYINVEREEWKLDTLCDLYETLTITQAVIFINTRRKVDWLTEKMHARDFTVSAMHGDMDQKERDVIMREFRSGSSRVLITTDLLARGIDVQQVSLVINYDLPTNRENYIHRIGRGGRFGRKGVAINMVTEEDKRTLRDIETFYNTSIEEMPLNVADLI

SEQ ID NO: 37 - protein, 479 aa, synthetic construct
MSDGFDRAPGAGRGRSRGLGRGGGGPEGGGFPNGAGPAERARHQPPQPKAPGFLQPPPLRQPRTTPPPGAQCEVPASPQRPSRPGALPEQTRPLRAPPSSQDKIPQQNSESAMAKPQVVVAPVLMSKLSVNAPEFYPSGYSSSYTESYEDGCEDYPTLSEYVQDFLNHLTEQPGSFETEIEQFAETLNGCVTTDDALQELVELIYQQATSIPNFSYMGARLCNYLSHHLTISPQSGNFRQLLLQRCRTEYEVKDQAAKGDEVTRKRFHAFVLFLGELYLNLEIKGTNGQVTRADILQVGLRELLNALFSNPMDDNLICAVKLLKLTGSVLEDAWKEKGKMDMEEIIQRIENVVLDANCSRDVKQMLLKLVELRSSNWGRVHATSTYREATPENDPNYFMNEPTFYTSDGVPFTAADPDYQEKYQELLEREDFFPDYEENGTDLSGAGDPYLDDIDDEMDPEIEEAYEKFCLESERKRKQ

SEQ ID NO: 38 - protein, 364 aa, synthetic construct
MSATSVDTQRTKGQDNKVQNGSLHQKDTVHDNDFEPYLTGQSNQSNSYPSMSDPYLSSYYPPSIGFPYSLNEAPWSTAGDPPIPYLTTYGQLSNGDHHFMHDAVFGQPGGLGNNIYQHRFNFFPENPAFSAWGTSGSQGQQTQSSAYGSSYTYPPSSLGGTVVDGQPGFHSDTLSKAPGMNSLEQGMVGLKIGDVSSSAVKTVGSVVSSVALTGVLSGNGGTNVNMPVSKPTSWAAIASKPAKPQPKMKTKSGPVMGGGLPPPPIKHNMDIGTWDNKGPVPKAPVPQQAPSPQAAPQPQQVAQPLPAQPPALAQPQYQSPQQPPQTRWVAPRNRNAAFGQSGGAGSDSNSPGNVQPNSAPSVES

SEQ ID NO: 39 - protein, 415 aa, synthetic construct
MSATSVDQRPKGQGNKVSVQNGSIHQKDAVNDDDFEPYLSSQTNQSNSYPPMSDPYMPSYYAPSIGFPYSLGEAAWSTAGDQPMPYLTTYGQMSNGEHHYIPDGVFSQPGALGNTPPFLGQHGFNFFPGNADFSTWGTSGSQGQSTQSSAYSSSYGYPPSSLGRAITDGQAGFGNDTLSKVPGISSIEQGMTGLKIGGDLTAAVTKTVGTALSSSGMTSIATNSVPPVSSAAPKPTSWAAIARKPAKPQPKLKPKGNVGIGGSAVPPPPIKHNMNIGTWDEKGSVVKAPPTQPVLPPQTIIQQPQPLIQPPPLVQSQLPQQQPQPPQPQQQQGPQPQAQPHQVQPQQQQLQNRWVAPRNRGAGFNQNNGAGSENFGLGVVPVSASPSSVEVHPVLEKLKAINNYNPKDFDWNLKN

SEQ ID NO: 40 - RNA, 31 nt, synthetic construct
  source 1..31: mol_type = other RNA
ggccagatctgagcctgggagctctctggcc

SEQ ID NO: 41 - RNA, 26 nt, synthetic construct
  source 1..26: mol_type = other RNA
ccaaaggctcttctcagagccaccca

SEQ ID NO: 42 - RNA, 21 nt, synthetic construct
  source 1..21: mol_type = other RNA
aaacatgaggattacccatgt

SEQ ID NO: 43 - RNA, 21 nt, synthetic construct
  source 1..21: mol_type = other RNA
aaacatgaggatcacccatgt

SEQ ID NO: 44 - RNA, 15 nt, synthetic construct
  source 1..15: mol_type = other RNA
gccctgaagaagggc

SEQ ID NO: 45 - RNA, 15 nt, synthetic construct
  source 1..15: mol_type = other RNA
gccctgaaaaagggc

SEQ ID NO: 46 - RNA, 25 nt, synthetic construct
  source 1..25: mol_type = other RNA
taaggagtttatatggaaaccctta

SEQ ID NO: 47 - RNA, 21 nt, synthetic construct
  source 1..21: mol_type = other RNA
aaacatgaggattacccatgt

SEQ ID NO: 48 - RNA, 21 nt, synthetic construct
  source 1..21: mol_type = other RNA
aaacatgaggatcacccatgt

SEQ ID NO: 49 - RNA, 20 nt, synthetic construct
  source 1..20: mol_type = other RNA
atgcatgtctaagacagcat

SEQ ID NO: 50 - RNA, 21 nt, synthetic construct
  source 1..21: mol_type = other RNA
aaacatgaggattacccatgt

SEQ ID NO: 51 - RNA, 21 nt, synthetic construct
  source 1..21: mol_type = other RNA
aaacatgaggatcacccatgt

SEQ ID NO: 52 - RNA, 23 nt, synthetic construct
  source 1..23: mol_type = other RNA
aaaacataaggaaaacctatgtt

SEQ ID NO: 53 - RNA, 28 nt, synthetic construct
  source 1..28: mol_type = other RNA
ggctcgtgtagctcattagctccgagcc

SEQ ID NO: 54 - RNA, 43 nt, synthetic construct
  source 1..43: mol_type = other RNA
ggcgtccctcccgaagctgcgcgctcggtcgaacaggacgacc

SEQ ID NO: 55 - DNA, 40 bp, synthetic construct
  source 1..40: mol_type = other DNA
ctctctgattttgaggaaaagaaattctgaagcccacatt

SEQ ID NO: 56 - RNA, 22 nt, synthetic construct
  source 1..22: mol_type = other RNA
ggccgaaatcccgaatgaggcc

SEQ ID NO: 57 - RNA, 21 nt, synthetic construct
  source 1..21: mol_type = other RNA
ggatgcctcccgagtgcatcc

SEQ ID NO: 58 - protein, 90 aa, synthetic construct
MDDPSFLTGRSTYAKRRRARRMNVCKCGAILHNNKDCRSSTISGHKLDRLRFVKEGRVALEGETPVYRTWVKWVETEYHINILETSDDEE

SEQ ID NO: 59 - protein, 76 aa, synthetic construct
MRVTLSSKPQALATPNKEEHGKRKKKGKGLGKKRDPCLRKYKDFCIHGECKYVKELRAPSCICHPGYHGERCHGLS

SEQ ID NO: 60 - protein, 282 aa, synthetic construct
MAVPETRPNHTIYINNLNEKIKKDELKKSLYAIFSQFGQILDILVSRSLKMRGQAFVIFKEVSSATNALRSMQGFPFYDKPMRIQYAKTDSDIIAKMKGTFVERDRKREKRKPKSQETPATKKAVQGGGATPVVGAVQGPVPGMPPMTQAPRIMHHMPGQPPYMPPPGMIPPPGLAPGQIPPGAMPPQQLMPGQMPPAQPLSENPPNHILFLTNLPEETNELMLSMLFNQFPGFKEVRLVPGRHDIAFVEFDNEVQAGAARDALQGFKITQNNAMKISFAKK

SEQ ID NO: 61 - protein, 314 aa, synthetic construct
QLLRDNTTGSNSSLNTSLPSPGAWPYSASDNSFTNVHSTSAKFPDYKSTWSPDPIGHNPTHLSNKMWKNHISSRNTTPLPRPPPGLTNPKPSSPWSSTAPRSVRGWGTQDSRLASASTWSDGGSVRPSYWLVLHNLTPQIDGSTLRTICMQHGPLLTFHLNLTQGTALIRYSTKQEAAKAQTALHMCVLGNTTILAEFATDDEVSRFLAQAQPPTPAATPSAPAAGWQSLETGQNQSDPVGPALNLFGGSTGLGQWSSSAGGSSGADLAGASLWGPPNYSSSLWGVPTVEDPHRMGSPAPLLPGDLLGGGSDSI

SEQ ID NO: 62 - protein, 579 aa, synthetic construct
MSAANPETPNSTISREASTQSSSAAASQGWVLPEGKIVPNTVFVGGIDARMDETEIGSCFGRYGSVKEVKIITNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQSAANPETPNSTISREASTQSSSAAASQGWVLPEGKIVPNTVFVGGIDARMDETEIGSCFGRYGSVKEVKIITNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQAYSAYPHSPGQVITGCQLLVYNYQEYPTYPDSAFQVTTGYQLPVYNYQPFPAYPRSPFQVTAGYQLPVYNYQAFPAYPNSPFQVATGYQFPVYNYQPFPAYPSSPFQVTAGYQLPVYNYQAFPAYPNSPFQVATGYQFPVYNYQAFPAYPNSPVQVTTGYQLPVYNYQAFPAYPSSPFQVTTGYQLPVYNYQAFPAYPNSAVQVTTGYQFHVYNYQMPPQCPVGEQRRNLWTEAYKWWYLVCLIQRRD

SEQ ID NO: 63 - protein, 489 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMAVPETRPNHTIYINNLNEKIKKDELKKSLYAIFSQFGQILDILVSRSLKMRGQAFVIFKEVSSATNALRSMQGFPFYDKPMRIQYAKTDSDIIAKMKGTFVERDRKREKRKPKSQETPATKKAVQGGGATPVVGAVQGPVPGMPPMTQAPRIMHHMPGQPPYMPPPGMIPPPGLAPGQIPPGAMPPQQLMPGQMPPAQPLSENPPNHILFLTNLPEETNELMLSMLFNQFPGFKEVRLVPGRHDIAFVEFDNEVQAGAARDALQGFKITQNNAMKISFAKKGSDYKDDDDK

SEQ ID NO: 64 - protein, 521 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSQLLRDNTTGSNSSLNTSLPSPGAWPYSASDNSFTNVHSTSAKFPDYKSTWSPDPIGHNPTHLSNKMWKNHISSRNTTPLPRPPPGLTNPKPSSPWSSTAPRSVRGWGTQDSRLASASTWSDGGSVRPSYWLVLHNLTPQIDGSTLRTICMQHGPLLTFHLNLTQGTALIRYSTKQEAAKAQTALHMCVLGNTTILAEFATDDEVSRFLAQAQPPTPAATPSAPAAGWQSLETGQNQSDPVGPALNLFGGSTGLGQWSSSAGGSSGADLAGASLWGPPNYSSSLWGVPTVEDPHRMGSPAPLLPGDLLGGGSDSIGSDYKDDDDK

SEQ ID NO: 65 - protein, 786 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSAANPETPNSTISREASTQSSSAAASQGWVLPEGKIVPNTVFVGGIDARMDETEIGSCFGRYGSVKEVKIITNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQSAANPETPNSTISREASTQSSSAAASQGWVLPEGKIVPNTVFVGGIDARMDETEIGSCFGRYGSVKEVKIITNRTGVSKGYGFVSFVNDVDVQKIVGSQIHFHGKKLKLGPAIRKQKLCARHVQPRPLVVNPPPPPQFQNVWRNPNTETYLQPQITPNPVTQHVQAYSAYPHSPGQVITGCQLLVYNYQEYPTYPDSAFQVTTGYQLPVYNYQPFPAYPRSPFQVTAGYQLPVYNYQAFPAYPNSPFQVATGYQFPVYNYQPFPAYPSSPFQVTAGYQLPVYNYQAFPAYPNSPFQVATGYQFPVYNYQAFPAYPNSPVQVTTGYQLPVYNYQAFPAYPSSPFQVTTGYQLPVYNYQAFPAYPNSAVQVTTGYQFHVYNYQMPPQCPVGEQRRNLWTEAYKWWYLVCLIQRRDGSDYKDDDDK

SEQ ID NO: 66 - protein, 531 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAEGSDYKDDDDK

SEQ ID NO: 67 - protein, 952 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAEGGSGGSGGSGGSGGSGGSLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTPGSDYKDDDDK

SEQ ID NO: 68 - protein, 952 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAEGGSGGSGGSGGSGGSGGSLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTPGSDYKDDDDK

SEQ ID NO: 69 - protein, 324 aa, synthetic construct
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE

SEQ ID NO: 70 - protein, 403 aa, synthetic construct
LHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 71 - protein, 403 aa, synthetic construct
LHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 72 - protein, 745 aa, synthetic construct
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAEGGSGGSGGSGGSGGSGGSLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 73 - protein, 745 aa, synthetic construct
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAEGGSGGSGGSGGSGGSGGSLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 74 - protein, 539 aa, synthetic construct
MAELTVEVRGSNGAFYKGFIKDVHEDSLTVVFENNWQPERQVPFNEVRLPPPPDIKKEISEGDEVEVYSRANDQEPCGWWLAKVRMMKGEFYVIEYAACDATYNEIVTFERLRPVNQNKTVKKNTFFKCTVDVPEDLREACANENAHKDFKKAVGACRIFYHPETTQLMILSASEATVKRVNILSDMHLRSIRTKLMLMSRNEEATKHLECTKQLAAAFHEEFVVREDLMGLAIGTHGSNIQQARKVPGVTAIELDEDTGTFRIYGESADAVKKARGFLEFVEDFIQVPRNLVGKVIGKNGKVIQEIVDKSGVVRVRIEGDNENKLPREDGMVPFVFVGTKESIGNVQVLLEYHIAYLKEVEQLRMERLQIDEQLRQIGSRSYSGRGRGRRGPNYTSGYGTNSELSNPSETESERKDELSDWSLAGEDDRDSRHQRDSRRRPGGRGRSVSGGRGRGGPRGGKSSISSVLKDPDSNPYSLLDNTESDQTADTDASESHHSTNRRRRSRRRRTDEDAVLMDGMTESDTASVNENGLGKRCD

SEQ ID NO: 75 - protein, 90 aa, synthetic construct
MADRAPRQPTSLGSSPQRCGSPPSAPPPPPDRGERIEDCLAPLCPPVVGGRGGCALSRFLPVDLSLLRSSRTAAVELVAGGVCRSPSQEN

SEQ ID NO: 76 - protein, 245 aa, synthetic construct
MNNKFDALKDDDSGDHDQNEENSTQKDGEKEKTERDKNQSSSKRKAVVPGPAEHPLQYNYTFWYSRRTPGRPTSSQSYEQNIKQIGTFASVEQFWRFYSHMVRPGDLTGHSDFHLFKEGIKPMWEDDANKNGGKWIIRLRKGLASRCWENLILAMLGEQFMVGEEICGAVVSVRFQEDIISIWNKTASDQATTARIRDTLRRVLNLPPNTIMEYKTHTDSIKMPGRLGPQRLLFQNLWKPRLNVP

SEQ ID NO: 77 - protein, 746 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMAELTVEVRGSNGAFYKGFIKDVHEDSLTVVFENNWQPERQVPFNEVRLPPPPDIKKEISEGDEVEVYSRANDQEPCGWWLAKVRMMKGEFYVIEYAACDATYNEIVTFERLRPVNQNKTVKKNTFFKCTVDVPEDLREACANENAHKDFKKAVGACRIFYHPETTQLMILSASEATVKRVNILSDMHLRSIRTKLMLMSRNEEATKHLECTKQLAAAFHEEFVVREDLMGLAIGTHGSNIQQARKVPGVTAIELDEDTGTFRIYGESADAVKKARGFLEFVEDFIQVPRNLVGKVIGKNGKVIQEIVDKSGVVRVRIEGDNENKLPREDGMVPFVFVGTKESIGNVQVLLEYHIAYLKEVEQLRMERLQIDEQLRQIGSRSYSGRGRGRRGPNYTSGYGTNSELSNPSETESERKDELSDWSLAGEDDRDSRHQRDSRRRPGGRGRSVSGGRGRGGPRGGKSSISSVLKDPDSNPYSLLDNTESDQTADTDASESHHSTNRRRRSRRRRTDEDAVLMDGMTESDTASVNENGLGKRCDGSDYKDDDDK

SEQ ID NO: 78 - protein, 297 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMADRAPRQPTSLGSSPQRCGSPPSAPPPPPDRGERIEDCLAPLCPPVVGGRGGCALSRFLPVDLSLLRSSRTAAVELVAGGVCRSPSQENGSDYKDDDDK

SEQ ID NO: 79 - protein, 452 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMNNKFDALKDDDSGDHDQNEENSTQKDGEKEKTERDKNQSSSKRKAVVPGPAEHPLQYNYTFWYSRRTPGRPTSSQSYEQNIKQIGTFASVEQFWRFYSHMVRPGDLTGHSDFHLFKEGIKPMWEDDANKNGGKWIIRLRKGLASRCWENLILAMLGEQFMVGEEICGAVVSVRFQEDIISIWNKTASDQATTARIRDTLRRVLNLPPNTIMEYKTHTDSIKMPGRLGPQRLLFQNLWKPRLNVPGSDYKDDDDK

SEQ ID NO: 80 - protein, 571 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSATSVDTQRTKGQDNKVQNGSLHQKDTVHDNDFEPYLTGQSNQSNSYPSMSDPYLSSYYPPSIGFPYSLNEAPWSTAGDPPIPYLTTYGQLSNGDHHFMHDAVFGQPGGLGNNIYQHRFNFFPENPAFSAWGTSGSQGQQTQSSAYGSSYTYPPSSLGGTVVDGQPGFHSDTLSKAPGMNSLEQGMVGLKIGDVSSSAVKTVGSVVSSVALTGVLSGNGGTNVNMPVSKPTSWAAIASKPAKPQPKMKTKSGPVMGGGLPPPPIKHNMDIGTWDNKGPVPKAPVPQQAPSPQAAPQPQQVAQPLPAQPPALAQPQYQSPQQPPQTRWVAPRNRNAAFGQSGGAGSDSNSPGNVQPNSAPSVESGSDYKDDDDK

SEQ ID NO: 81 - protein, 424 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMATVEPETTPTPNPPTTEEEKTESNQEVANPEHYIKHPLQNRWALWFFKNDKSKTWQANLRLISKFDTVEDFWALYNHIQLSSNLMPGCDYSLFKDGIEPMWEDEKNKRGGRWLITLNKQQRRSDLDRFWLETLLCLIGESFDDYSDDVCGAVVNVRAKGDKIAIWTTECENREAVTHIGRVYKERLGLPPKIVIGYQSHADTATKSGSTTKNRFVVGSDYKDDDDK

SEQ ID NO: 82 - protein, 613 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGGSGGSGGSGGSGGSGGSLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTPKRPAATKKAGQAKKKKGSDYKDDDDK

SEQ ID NO: 83 - DNA, 22 bp, synthetic construct
  source 1..22: mol_type = other DNA
cgtccaacactgctactcctca

SEQ ID NO: 84 - DNA, 22 bp, synthetic construct
  source 1..22: mol_type = other DNA
gcctttggtgagagtgaagagc

SEQ ID NO: 85 - DNA, 23 bp, synthetic construct
  source 1..23: mol_type = other DNA
catcactgccacccagaagactg

SEQ ID NO: 86 - DNA, 23 bp, synthetic construct
  source 1..23: mol_type = other DNA
atgccagtgagcttcccgttcag

SEQ ID NO: 87 - DNA, 40 bp, synthetic construct
  source 1..40: mol_type = other DNA
tgacagcccacatggcattccacttatcactggcatcctt

SEQ ID NO: 88 - DNA, 40 bp, synthetic construct
  source 1..40: mol_type = other DNA
gtggctgggagttgggctcgggatcagaggagcggtgcct

SEQ ID NO: 89 - DNA, 40 bp, synthetic construct
  source 1..40: mol_type = other DNA
cagaagaacaggaacagagccaggacgctgaagatgacag

SEQ ID NO: 90 - DNA, 40 bp, synthetic construct
  source 1..40: mol_type = other DNA
atcacatagatgataccactgaggagggctaggggaaagg

SEQ ID NO: 91 - DNA, 40 bp, synthetic construct
  source 1..40: mol_type = other DNA
gagacgaagagcaacactagcaccgcgatgtgcaggaaca

SEQ ID NO: 92 - DNA, 40 bp, synthetic construct
  source 1..40: mol_type = other DNA
cgatgtgcaggaacaggatccccaacaagagtaggagcat

SEQ ID NO: 93 - DNA, 40 bp, synthetic construct
  source 1..40: mol_type = other DNA
aatccagtgatgtaaaaccggccgcctttggtgagagtga

SEQ ID NO: 94 - DNA, 40 bp, synthetic construct
  source 1..40: mol_type = other DNA
atgccactcactgtgcctcactgtgtagatggccgctgca

SEQ ID NO: 95 - DNA, 40 bp, synthetic construct
  source 1..40: mol_type = other DNA
tcagtcgtgtgtccattgcccacgagccattggctgacga

SEQ ID NO: 96 - DNA, 40 bp, synthetic construct
  source 1..40: mol_type = other DNA
agactgcagccattcgctcactgatgaggagtagcagtgt

SEQ ID NO: 97 - DNA, 40 bp, synthetic construct
  source 1..40: mol_type = other DNA
ctgaagatgacagacaggatcatggtggcctggacagact

SEQ ID NO: 98 - DNA, 40 bp, synthetic construct
  source 1..40: mol_type = other DNA
gcgaagccataggagtagtcagtgttgacatgccactcac

SEQ ID NO: 99 - DNA, 40 bp, synthetic construct
  source 1..40: mol_type = other DNA
cgcgctcagagcctagacggacggtgcgtcgtcgggcgcc

SEQ ID NO: 100 - DNA, 40 bp, synthetic construct
  source 1..40: mol_type = other DNA
ccacagggagggaggaaggaaaccagagaacaaaaccaac

SEQ ID NO: 101 - DNA, 40 bp, synthetic construct
  source 1..40: mol_type = other DNA
ttggtggccaatacaagtcatcgccagacagtccttggag

SEQ ID NO: 102 - DNA, 40 bp, synthetic construct
  source 1..40: mol_type = other DNA
cagttggtataaaatcagagaaagcaagcaaaaacaagaa

SEQ ID NO: 103 - DNA, 40 bp, synthetic construct
  source 1..40: mol_type = other DNA
gatttatttattgtagtaataacaacagcctagctaggta

SEQ ID NO: 104 - DNA, 40 bp, synthetic construct
  source 1..40: mol_type = other DNA
gatattatatacatcttcaatcaacagcaatccccactca

SEQ ID NO: 105 - DNA, 40 bp, synthetic construct
  source 1..40: mol_type = other DNA
caaaacaaagatactgagcttggttatagtgttaggatgt

SEQ ID NO: 106 - DNA, 40 bp, synthetic construct
  source 1..40: mol_type = other DNA
cttgtctctgacttctcatttaaacgtgtggccccagtac

SEQ ID NO: 107 - DNA, 40 bp, synthetic construct
  source 1..40: mol_type = other DNA
tgcactgggtcacccatagtgtaaaggtcttcaactgtct

SEQ ID NO: 108 - DNA, 40 bp, synthetic construct
  source 1..40: mol_type = other DNA
gctcagcggagtttctgcccggccaaacagcgtaacccct

SEQ ID NO: 109 - DNA, 40 bp, synthetic construct
  source 1..40: mol_type = other DNA
ctggcggcaagttctgctcagcggagtttctgcccggcca

SEQ ID NO: 110 - DNA, 40 bp, synthetic construct
  source 1..40: mol_type = other DNA
gggtgaagagttggcagaagaacaggaacagagacagaat

SEQ ID NO: 111 - DNA, 41 bp, synthetic construct
  source 1..41: mol_type = other DNA
ttttcccttcctcccttccctatgtacgctcagagcctcag

SEQ ID NO: 112 - DNA, 40 bp, synthetic construct
  source 1..40: mol_type = other DNA
cctatgtacgctcagagcctcagacagaccgtctgggcgc

SEQ ID NO: 113 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 114 - protein, 344 aa, synthetic construct
MQLEIKVALNFIISYLYNKLPRRRADLFGEELERLLKKKYEGHWYPEKPLKGSGFRCVHIGEMVDPVVELAAKRSGLAVEDVRANVPEELSVWIDPFEVSYQIGEKGAVKVLYLDDSEGCGAPELDKEIKSSFNPDAQVFVPIGSQDSSLSNSPSPSFGQSPSPTFIPRSAQPITFTTASFAATKFGSTKMKKGGGAASGGGVASSGAGGQQPPQQPRMARSPTNSLLKHKSLSLSMHSLNFITANPAPQSQLSPNAKEFVYNGGGSPSLFFDAADGQGSGTPGPFGGSGAGTCNSSSFDMAQVFGGGANSLFLEKTPFVEGLSYNLNTMQYPSQQFQPVVLAN

SEQ ID NO: 115 - protein, 120 aa, synthetic construct
MQDGMYQRFLRQHVHPEETGGSDRYCNLMMQRRKMTLYHCKRFNTFIHEDIWNIRSICSTTNIQCKNGKMNCHEGVVKVTDCRDTGSSRAPNCRYRAIASTRRVVIACEGNPQVPVHFDG

SEQ ID NO: 116 - protein, 406 aa, synthetic construct
WKPQSSHWGAALKDLHRIYRPMIGKLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 117 - protein, 123 aa, synthetic construct
ETSAEKFERQHMDADSSPASNSNYCNKMMKSRSMTTDRCKPVNTFIHEPLADVQAVCSQTNVACKNGQTNCYQSKAAMRITDCRETGSSKYPDCAYTTTEEEKHIIVACEGNPYVPVHFDATV

SEQ ID NO: 118 - protein, 129 aa, synthetic construct [sequence omitted]

SEQ ID NO: 119 - protein, 114 aa, synthetic construct [sequence omitted]

SEQ ID NO: 120 - protein, 137 aa, synthetic construct
MSSLIRRVISTAKAPGAIGPYSQAVLVDRTIYISGQIGMDPSSGQLVSGGVAEEAKQALKNMGEILKAAGCDFTNVVKTTVLLADINDFNTVNEIYKQYFKSNFPARAAYQVAALPKGSRIEIEAVAIQGPLTTASL

SEQ ID NO: 121 - protein, 199 aa, synthetic construct
MENFRKVRSEEAPAGCGAEGGGPGSGPFADLAPGAVHMRVKEGSKIRNLMAFATASMAQPATRAIVFSGCGRATTKTVTCAEILKRRLAGLHQVTRLRYRSVREVWQSLPPGPTQGQTPGEPAASLSVLKNVPGLAILLSKDALDPRQPGYQPPNPHPGPSSPPAAPASKRSLGEPAAGEGSAKRSQPEPGVADEDQTA

SEQ ID NO: 122 - DNA, 40 bp, synthetic construct
  source 1..40: mol_type = other DNA
taatgaattcctttacaccacactgtcgtcgaatggccac

SEQ ID NO: 123 - DNA, 40 bp, synthetic construct
  source 1..40: mol_type = other DNA
tcttctcagccactgttgccacaccatgcaccactccctc

SEQ ID NO: 124 - DNA, 40 bp, synthetic construct
  source 1..40: mol_type = other DNA
agaaggcatttcataagcctcattgtcaggatccacaggc

SEQ ID NO: 125 - DNA, 40 bp, synthetic construct
  source 1..40: mol_type = other DNA
ttaacatcgtagattgaagccacaaaatccacagcacaca

SEQ ID NO: 126 - DNA, 40 bp, synthetic construct
  source 1..40: mol_type = other DNA
tttcaataattaatcacagccacttaaggaaccagtgcat

SEQ ID NO: 127 - DNA, 40 bp, synthetic construct
  source 1..40: mol_type = other DNA
cgaatattatttattgtcagaaaggtacagcattcacacc

SEQ ID NO: 128 - DNA, 40 bp, synthetic construct
  source 1..40: mol_type = other DNA
gcaacatttaaaggaggccatgaaatttttatcagtctag

SEQ ID NO: 129 - DNA, 40 bp, synthetic construct
  source 1..40: mol_type = other DNA
cagagtttttcaccatcaagtttattttcagcacccatgg

SEQ ID NO: 130 - DNA, 40 bp, synthetic construct
  source 1..40: mol_type = other DNA
ctactagtgacttttagaaatgactatgccccagaataat

SEQ ID NO: 131 - DNA, 40 bp, synthetic construct
  source 1..40: mol_type = other DNA
tagatttaaaatatatcctaaccgccactttctaaccttc

SEQ ID NO: 132 - DNA, 21 bp, synthetic construct
  source 1..21: mol_type = other DNA
caacagtggctgagaagacca

SEQ ID NO: 133 - DNA, 22 bp, synthetic construct
  source 1..22: mol_type = other DNA
gctccttcttcattcttgccca

SEQ ID NO: 134 - DNA, 18 bp, synthetic construct
  source 1..18: mol_type = other DNA
caccaactgggacgacat

SEQ ID NO: 135 - DNA, 18 bp, synthetic construct
  source 1..18: mol_type = other DNA
acagcctggatagcaacg

SEQ ID NO: 136 - protein, 636 aa, synthetic construct
MNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTV

SEQ ID NO: 137 - protein, 889 aa, synthetic construct
EPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 138 - protein, 394 aa, synthetic construct
EPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAK

SEQ ID NO: 139 - protein, 283 aa, synthetic construct
MQTDSLSPSPNPVSPVPLNNPTSAPRYGTVIPNRIFVGGIDFKTNESDLRKFFSQYGSVKEVKIVNDRAGVSKGYGFVTFETQEDAQKILQEAEKLNYKDKKLNIGPAIRKQQVGIPRSSIMPAAGTMYLTTSTGYPYTYHNGVAYFHTPEVTSVPPPWPSRSVCSSPVMVAQPIYQQPAYHYQATTQYLPGQWQWSVPQPSASSAPFLYLQPSEVIYQPVEIAQDGGCVPPPLSLMETSVPEPYSDHGVQATYHQVYAPSAITMPAPVMQPEPIKTVWSIHY

SEQ ID NO: 140 - protein, 595 aa, synthetic construct [sequence omitted]

SEQ ID NO: 141 - protein, 331 aa, synthetic construct
MDTGVIEGGLNVTLTIRLLMHGKEVGSIIGKKGESVKKMREESGARINISEGNCPERIITLAGPTNAIFKAFAMIIDKLEEDISSSMTNSTAASRPPVTLRLVVPASQCGSLIGKGGCKIKEIRESTGAQVQVAGDMLPNSTERAITIAGIPQSIIECVKQICVVMLESPPKGVTIPYRPKPSSSPVIFAGGQAYTIQGQYAIPQPDLTKLHQLAMQQSHFPMTHGNTGFSGIESSSPEVKGYWAGLDASAQTTSHELTIPNDLIGCIIGRQGAKINEIRQMSGAQIKIANPVEGSTDRQVTITGSAASISLAQYLINVRLSSETGGMGSS

SEQ ID NO: 142 - protein, 326 aa, synthetic construct
MSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPITVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

SEQ ID NO: 143 - protein, 305 aa, synthetic construct
MDLSGVKKKSLLGVKENNKKSSTRAPSPTKRKDRSDEKSKDRSKDKGATKESSEKDRGRDKTRKRRSASSGSSSTRSRSSSTSSSGSSTSTGSSSGSSSSSASSRSGSSSTSRSSSSSSSSGSPSPSRRRHDNRRRSRSKSKPPKRDEKERKRRSPSPKPTKVHIGRLTRNVTKDHIMEIFSTYGKIKMIDMPVERMHPHLSKGYAYVEFENPDEAEKALKHMDGGQIDGQEITATAVLAPWPRPPPRRFSPPRRMLPPPPMWRRSPPRMRRRSRSPRRRSPVRRRSRSPGRRRHRSRSSSNSSR

SEQ ID NO: 144 - DNA, 40 bp, synthetic construct
  source 1..40: mol_type = other DNA
gccggtagaactacgtaagctcagaagaggccacactgtt

SEQ ID NO: 145 - DNA, 40 bp, synthetic construct
  source 1..40: mol_type = other DNA
gagtatgacacgacagtttaaagaactacaagccctcaga

SEQ ID NO: 146 - DNA, 40 bp, synthetic construct
  source 1..40: mol_type = other DNA
taaattcttcattttaccagcaactgctgacatcaaagtc

SEQ ID NO: 147 - DNA, 40 bp, synthetic construct
  source 1..40: mol_type = other DNA
ttaatcatattcaaccactcacattttggctatgttaagt

SEQ ID NO: 148 - protein, 445 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSDYKDDDDK

SEQ ID NO: 149 - protein, 398 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAAGSDYKDDDDK

SEQ ID NO: 150 - protein, 492 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMPAATVDHSQRICEVWACNLDEEMKKIRQVIRKYNYVAMDTEFPGVVARPIGEFRSNADYQYQLLRCNVDLLKIIQLGLTFMNEQGEYPPGTSTWQFNFKFNLTEDMYAQDSIELLTTSGIQFKKHEEEGIETQYFAELLMTSGVVLCEGVKWLSFHSGYDFGYLIKILTNSNLPEEELDFFEILRLFFPVIYDVKYLMKSCKNLKGGLQEVAEQLELERIGPQHQAGSDSLLTGMAFFKMREMFFEDHIDDAKYCGHLYGLGSGSSYVQNGTGNAYEEEANKQSGSDYKDDDDK

SEQ ID NO: 151 - protein, 533 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMDLTAIYESLLSLSPDVPVPSDHGGTESSPGWGSSGPWSLSPSDSSPSGVTSRLPGRSTSLVEGRSCGWVPPPPGFAPLAPRLGPELSPSPTSPTATSTTPSRYKTELCRTFSESGRCRYGAKCQFAHGLGELRQANRHPKYKTELCHKFYLQGRCPYGSRCHFIHNPSEDLAAPGHPPVLRQSISFSGLPSGRRTSPPPPGLAGPSLSSSSFSPSSSPPPPGDLPLSPSAFSAAPGTPLARRDPTPVCCPSCRRATPISVWGPLGGLVRTPSVQSLGSDPDEYASSGSSLGGSDSPVFEAGVFAPPQPVAAPRRLPIFNRISVSEGSDYKDDDDK

SEQ ID NO: 152 - protein, 551 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMQLEIKVALNFIISYLYNKLPRRRADLFGEELERLLKKKYEGHWYPEKPLKGSGFRCVHIGEMVDPVVELAAKRSGLAVEDVRANVPEELSVWIDPFEVSYQIGEKGAVKVLYLDDSEGCGAPELDKEIKSSFNPDAQVFVPIGSQDSSLSNSPSPSFGQSPSPTFIPRSAQPITFTTASFAATKFGSTKMKKGGGAASGGGVASSGAGGQQPPQQPRMARSPTNSLLKHKSLSLSMHSLNFITANPAPQSQLSPNAKEFVYNGGGSPSLFFDAADGQGSGTPGPFGGSGAGTCNSSSFDMAQVFGGGANSLFLEKTPFVEGLSYNLNTMQYPSQQFQPVVLANGSDYKDDDDK

SEQ ID NO: 153 - protein, 327 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMQDGMYQRFLRQHVHPEETGGSDRYCNLMMQRRKMTLYHCKRFNTFIHEDIWNIRSICSTTNIQCKNGKMNCHEGVVKVTDCRDTGSSRAPNCRYRAIASTRRVVIACEGNPQVPVHFDGGSDYKDDDDK

SEQ ID NO: 154 - protein, 613 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSWKPQSSHWGAALKDLHRIYRPMIGKLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGCGSDYKDDDDK

SEQ ID NO: 155 - protein, 330 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSETSAEKFERQHMDADSSPASNSNYCNKMMKSRSMTTDRCKPVNTFIHEPLADVQAVCSQTNVACKNGQTNCYQSKAAMRITDCRETGSSKYPDCAYTTTEEEKHIIVACEGNPYVPVHFDATVGSDYKDDDDK

SEQ ID NO: 156 - protein, 388 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVGGSDYKDDDDK

SEQ ID NO: 157 - protein, 336 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTGSDYKDDDDK

SEQ ID NO: 158 - protein, 321 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSCDIPQSTNCGGNVYSNDDINTAIQGALDDVANGDRPDNYPHQYYDEASEDITLCCGSGPWSEFPLVYNGPYYSSRDNYVSPGPDRVIYQTNTGEFCATVTHTGAASYDGFTQCSGSDYKDDDDK

SEQ ID NO: 159 - protein, 344 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSSLIRRVISTAKAPGAIGPYSQAVLVDRTIYISGQIGMDPSSGQLVSGGVAEEAKQALKNMGEILKAAGCDFTNVVKTTVLLADINDFNTVNEIYKQYFKSNFPARAAYQVAALPKGSRIEIEAVAIQGPLTTASLGSDYKDDDDK

SEQ ID NO: 160 - protein, 406 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMENFRKVRSEEAPAGCGAEGGGPGSGPFADLAPGAVHMRVKEGSKIRNLMAFATASMAQPATRAIVFSGCGRATTKTVTCAEILKRRLAGLHQVTRLRYRSVREVWQSLPPGPTQGQTPGEPAASLSVLKNVPGLAILLSKDALDPRQPGYQPPNPHPGPSSPPAAPASKRSLGEPAAGEGSAKRSQPEPGVADEDQTAGSDYKDDDDK

SEQ ID NO: 161 - protein, 613 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSASQDSRSRDNGPDGMEPEGVIESNWNEIVDSFDDMNLSESLLRGIYAYGFEKPSAIQQRAILPCIKGYDVIAQAQSGTGKTATFAISILQQIELDLKATQALVLAPTRELAQQIQKVVMALGDYMGASCHACIGGTNVRAEVQKLQMEAPHIIVGTPGRVFDMLNRRYLSPKYIKMFVLDEADEMLSRGFKDQIYDIFQKLNSNTQVVLLSATMPSDVLEVTKKFMRDPIRILVKKEELTLEGIRQFYINVEREEWKLDTLCDLYETLTITQAVIFINTRRKVDWLTEKMHARDFTVSAMHGDMDQKERDVIMREFRSGSSRVLITTDLLARGIDVQQVSLVINYDLPTNRENYIHRIGRGGRFGRKGVAINMVTEEDKRTLRDIETFYNTSIEEMPLNVADLIGSDYKDDDDK

SEQ ID NO: 162 - protein, 843 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTVGSDYKDDDDK

SEQ ID NO: 163 - protein, 1096 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHNGSDYKDDDDK

SEQ ID NO: 164 - protein, 601 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKGSDYKDDDDK

SEQ ID NO: 165 - protein, 490 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMQTDSLSPSPNPVSPVPLNNPTSAPRYGTVIPNRIFVGGIDFKTNESDLRKFFSQYGSVKEVKIVNDRAGVSKGYGFVTFETQEDAQKILQEAEKLNYKDKKLNIGPAIRKQQVGIPRSSIMPAAGTMYLTTSTGYPYTYHNGVAYFHTPEVTSVPPPWPSRSVCSSPVMVAQPIYQQPAYHYQATTQYLPGQWQWSVPQPSASSAPFLYLQPSEVIYQPVEIAQDGGCVPPPLSLMETSVPEPYSDHGVQATYHQVYAPSAITMPAPVMQPEPIKTVWSIHYGSDYKDDDDK

SEQ ID NO: 166 - protein, 802 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATVGSDYKDDDDK

SEQ ID NO: 167 - protein, 538 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMDTGVIEGGLNVTLTIRLLMHGKEVGSIIGKKGESVKKMREESGARINISEGNCPERIITLAGPTNAIFKAFAMIIDKLEEDISSSMTNSTAASRPPVTLRLVVPASQCGSLIGKGGCKIKEIRESTGAQVQVAGDMLPNSTERAITIAGIPQSIIECVKQICVVMLESPPKGVTIPYRPKPSSSPVIFAGGQAYTIQGQYAIPQPDLTKLHQLAMQQSHFPMTHGNTGFSGIESSSPEVKGYWAGLDASAQTTSHELTIPNDLIGCIIGRQGAKINEIRQMSGAQIKIANPVEGSTDRQVTITGSAASISLAQYLINVRLSSETGGMGSSGSDYKDDDDK

SEQ ID NO: 168 - protein, 533 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPITVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHKGSDYKDDDDK

SEQ ID NO: 169 - protein, 512 aa, synthetic construct
MGIINTLQKYYCRVRGGRCAVLSCLPKEEQIGKCSTRGRKCCRRKKGGSGGSGGSGGSGGSGGSMAVPETRPNHTIYINNLNSKIKKDELKKSLYAIFSQFGQILDILVPRQRTPRGQAFVIFKEVSSATNALRSMQGFPFYDKPMRIQYAKTDKRIPAKMKGTFVGSLQLPPLERLTLGGSGGSGGSGGSGGSGGSMDLSGVKKKSLLGVKENNKKSSTRAPSPTKRKDRSDEKSKDRSKDKGATKESSEKDRGRDKTRKRRSASSGSSSTRSRSSSTSSSGSSTSTGSSSGSSSSSASSRSGSSSTSRSSSSSSSSGSPSPSRRRHDNRRRSRSKSKPPKRDEKERKRRSPSPKPTKVHIGRLTRNVTKDHIMEIFSTYGKIKMIDMPVERMHPHLSKGYAYVEFENPDEAEKALKHMDGGQIDGQEITATAVLAPWPRPPPRRFSPPRRMLPPPPMWRRSPPRMRRRSRSPRRRSPVRRRSRSPGRRRHRSRSSSNSSRGSDYKDDDDK

(19) United States
(12) Patent Application Publication
(10) Pub. No.: US 2025/0283062 A1
(43) Pub. Date: Sep. 11, 2025

(54) SYSTEMS AND METHODS FOR MODULATING RNA

(71) Applicant: THE UNIVERSITY OF CHICAGO, Chicago, IL (US)

(72) Inventors: Bryan DICKINSON, Chicago, IL (US); Krysten JONES, Chicago, IL (US); Riley William SINNOTT, Chicago, IL (US)

(73) Assignee: THE UNIVERSITY OF CHICAGO, Chicago, IL (US)

(21) Appl. No.: 18/861,526

(22) PCT Filed: May 1, 2023
(86) PCT No.: PCT/US2023/066438

§ 371 Date: Oct. 29, 2024

Related U.S. Application Data
(60) Provisional application No. 63/337,481, filed on May 2, 2022.

(51) Int. Cl. C12N 9/22; C12N 15/88

(52) U.S. Cl. CPC C12N 9/22; C12N 15/88

(57) ABSTRACT

Aspects of the disclosure relate to a Effector system comprising at least one of each: i) a RNA hairpin binding domain; ii) a RNA targeting molecule comprising a RNA targeting region and at least one hairpin structure, wherein the hairpin structure of the RNA targeting molecule specifically binds to i; and iii) a Effector domain.

### CROSS-REFERENCE TO RELATED APPLICATIONS

This application claims the benefit of priority to U.S. Provisional Patent Application Ser. No. 63/337,481, filed May 2, 2022, which is hereby incorporated by reference in its entirety.

### STATEMENT OF GOVERNMENT SUPPORT

This invention was made with government support under grant number GM119840 awarded by the National Institutes of Health. The government has certain rights in the invention.

### SEQUENCE LISTING

The application contains a Sequence Listing prepared in compliance with ST.26 format and is hereby incorporated by reference in its entirety. Said Sequence Listing, created on May 1, 2023 is named ARCDP0734WO.xml and is 194,423 bytes in size.

### BACKGROUND OF THE INVENTION

### 1. Field of the Invention

The present invention relates generally to the field of chemistry and medicine. More particularly, it concerns the use of a system for modulating RNA.

### 2. Description of Related Art

Programmable nucleic acid-binding proteins have revolutionized genome studies and editing technologies (Chandrasegaran and Carroll, 2016; Filipovska et al., 2011; Gootenberg et al., 2018; Hilton et al., 2015; Joung and Sander, 2012; Kearns et al., 2015; Strutt et al., 2018) and are opening up new therapeutic opportunities to treat human diseases (Liao et al., 2017; Monteys et al., 2017). In particular, the CRISPR/Cas9 system, which evolved as a bacterial immune defense mechanism, has transformed the ability to study and manipulate cellular DNA site-specifically (Cong et al., 2013; Jiang et al., 2013; O'Connell et al., 2014; Wiedenheft et al., 2012). A key advantage of CRISPR/Cas systems compared to previous methods (Desjarlais and Berg, 1993; Hockemeyer et al., 2011; Joung and Sander, 2012; Schierling et al., 2012) is that they are easily programmable to target virtually any locus of interest. The CRISPR/Cas system is a ribonucleoprotein complex that uses base pair interactions of a displayed guide RNA (gRNA) to interact with a target nucleic acid sequence. The simple nature of base pair-guided targeting opens up the possibility to program systems to interact with a defined nucleic acid sequence by simply changing the nucleic acid sequence on the guiding strand.

While targeting DNA directly will have profound clinical ramifications, diseases that involve subtle alterations to many genes will be challenging to target using DNA editing technologies (Fuxman Bass et al., 2015). Additionally, the potential side effects or risks of permanent genetic alteration might not be tolerated. For example, the genes one may want to target to activate an enhanced wound healing response are likely targets that could pose a risk for cancer development, making permanent DNA-based strategy risky. Targeting information flow at the RNA level presents several opportunities for therapeutic intervention, including but not limited to the ability to halt treatment if side effects emerge, the ability to target genes that would be too risky to alter at the DNA level, and the ability to manipulate gene expression without permanent alterations to the host genome. While inhibiting or enhancing transcription at the genome level provides one possibility for controlling gene expression (Du et al., 2017; Fuxman Bass et al., 2015; Qi et al., 2013), recently discovered RNA epitranscriptomic regulatory mechanisms offer a broad range of processes to target, including editing, degradation, transport, and translation of RNA transcripts (Nishikura, 2010; Roundtree et al., 2017; Zhao et al., 2017). Although the mechanisms and consequences of this epitranscriptomic regulatory layer are just beginning to be uncovered, it is apparent that the information flow through RNA is tightly regulated, offering many new opportunities for both basic research discoveries as well as therapeutic development.

Programmable RNA-targeting tools analogous to the dCas9 DNA-targeting systems hold great promise for studying the mechanisms of epitranscriptomic regulation and for therapeutic applications. The current tools for RNA targeting involve the delivery of large complexes and pose immunogenicity issues. From a basic science perspective, the large size of the delivery vehicle could lead to potential perturbations to the RNA under interrogation, convoluting the study of RNA regulatory mechanisms. From a translational perspective, the large size presents challenges for viral packaging or direct protein delivery. Additionally, while DNA-editing therapies will likely consist of a one-time, irreversible treatment, RNA-targeting therapies will need to be continually administered, making delivery concerns especially important. Moreover, it was recently discovered that 85% of people already have circulating antibodies to CRISPR/Cas proteins (Kim et al., 2018; Wagner et al., 2018), suggesting immunogenicity issues may prove problematic in clinical applications. Therefore, there is a need in the art for improved systems that can target RNA and be delivered efficiently without activating an immune response.

### SUMMARY OF THE INVENTION

To overcome the large size and microbial-derived nature of current RNA-targeting systems, the inventors present a CRISPR/Cas-inspired RNA targeting system (CIRTS), a general method for engineering programmable RNA effector systems. Similar to CRISPR/Cas-based systems, CIRTS is a ribonucleoprotein complex that uses Watson-Crick-Franklin base pair interactions to deliver protein cargo site-selectively in the transcriptome. The inventors show they can easily engineer CIRTS that deliver a range of regulatory proteins to transcripts, including nucleases for degradation, deadenylation regulatory machinery for degradation, or translational activation machinery for enhanced protein production. However, CIRTS complexes are up to 5-fold smaller than the smallest current CRISPR/Cas systems and can be engineered entirely from human parts.

Aspects of the disclosure relate to a polypeptide comprising an effector domain wherein the effector domain comprises the amino acid sequence of one of SEQ ID NOs: 19-39, 60-62, 69-76, 113-121, or 136-143 or comprises an amino acid sequence with at least 80% sequence identity to one of SEQ ID NOs: 19-39, 60-62, 69-76, 113-121, or 136-143 or a functional portion thereof; and wherein the polypeptide further comprises one or more of a stabilizer domain, linker, RNA hairpin binding domain, nuclear export signal, and molecular tag. In some aspects, the effector domain comprises an amino acid sequence having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOs: 19-39, 60-62, 69-76, 113-121, or 136-143 or a functional portion thereof. Further aspects relate to a polypeptide comprising an effector domain, wherein the effector domain comprises a fusion between at least two polypeptides, wherein each polypeptide is independently selected from a polypeptide comprising an amino acid sequence of one of SEQ ID NOs: 22-24, 28-39, 60-62, 69-76, 113-121, or 136-143 or a polypeptide having an amino acid sequence with at least 80% sequence identity to one of SEQ ID NOs: 22-24, 28-39, 60-62, 69-76, 113-121, or 136-143 or a functional portion thereof. In some aspects, the effector domain comprises a fusion between at least two polypeptides, wherein each polypeptide is independently selected from a polypeptide that has an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOs: 22-24, 28-39, 60-62, 69-76, 113-121, or 136-143 or a functional portion thereof.

Further aspects of the disclosure relate to a polypeptide comprising an effector domain, wherein the effector domain comprises a fusion between exactly, at least, or at most 2, 3, 4, 5, or 6 polypeptides, wherein each polypeptide is independently selected from a polypeptide comprising an amino acid sequence of one of SEQ ID NOs: 22-24, 28-39, 60-62, 69-76, 113-121, or 136-143 or a polypeptide having an amino acid sequence with at least 80% sequence identity to one of SEQ ID NOs: 22-24, 28-39, 60-62, 69-76, 113-121, or 136-143 or a functional portion thereof. In some aspects, the effector domain comprises a fusion between exactly, at least, or at most 2, 3, 4, 5, or 6 polypeptides, wherein each polypeptide is independently selected from a polypeptide that has an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOs: 22-24, 28-39, 60-62, 69-76, 113-121, or 136-143 or a functional portion thereof.

Further aspects relate to nucleic acids encoding the polypeptides of the disclosure. Nucleic acids also include a nucleic acid encoding a RNA targeting molecule comprising a RNA targeting region and at least one hairpin structure, wherein the hairpin structure of the RNA targeting molecule specifically binds to the RNA hairpin binding domain of the polypeptide. Also disclosed are expression vectors comprising the nucleic acids of the disclosure and host cells comprising the nucleic acid or expression vector of the disclosure. Yet further aspects relate to a delivery vehicle comprising a polypeptide, nucleic acid, or expression vector of the disclosure. Also described are compositions comprising a polypeptide, nucleic acid, expression vector, host cell, or delivery vehicle of the disclosure. Further aspects relate to a method of making a polypeptide comprising transferring a nucleic acid or expression vector of the disclosure into a cell. The method may further comprise isolating the expressed polypeptide. Further aspects relate to a method of modulating at least one target RNA comprising contacting the target RNA with a polypeptide, delivery vehicle, or composition of the disclosure. Also described is a method for modulating at least one target RNA in a subject, the method comprising administering the polypeptide, delivery vehicle, or composition of the disclosure to the subject.

Further aspects relate to a cell or progeny thereof comprising modulated target RNA, wherein the target RNA has been modulated according to the methods of the disclosure. Yet further aspects relate to a multicellular organism comprising one or more cells of the disclosure. Also described is a plant or animal comprising one or more cells or the disclosure. Further aspects relate to a kit comprising a polypeptide, nucleic acid, expression vector, host cell, delivery vehicle, or composition of the disclosure.

Polypeptides provided and/or utilized in accordance with the present disclosure may comprise or further comprise one or more linkers. The linkers may be between different regions or domains of the polypeptide. In some aspects, the effector domain comprises a linker between the two fused polypeptides. For example, the linker may be tween an effector domain and a molecular tag, between an effector domain and a RNA hairpin binding domain, and/or between a stabilizer domain and an RNA hairpin binding domain. In some aspects, the linker comprises or consists of glycine and serine residues. The linker may comprise, may comprise at least, or may comprise at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 44, 45, 46, 47, 48, 49, or 50 amino acids (or any derivable range therein). In some aspects the linker comprises or consists of GS. In some aspects, the linker has an amino acid sequence that comprises or consists of SEQ ID NO:15. Alternative linkers, including glycine-serine linkers, that are employable in methods, compositions, delivery systems, polypeptides, and nucleic acids of the disclosure are known in the art and described herein. The polypeptides may be ones that do not have a linker between different regions or domains of the polypeptide. The polypeptides may exclude having a linker between an effector domain and a molecular tag, between an effector domain and a RNA hairpin binding domain, and/or between a stabilizer domain and an RNA hairpin binding domain.

In some aspects, the polypeptide has an amino acid sequence that is or comprises one of SEQ ID NOs: 32-39 or 69-76, or a functional portion thereof, or an amino acid sequence that shares at least 80% sequence identity to one of SEQ ID NOs: 32-39 or 69-76, or a functional portion thereof. In some aspects, the polypeptide is independently selected from a polypeptide having, having at least, or having at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOs: 32-39 or 69-76, or a functional portion thereof.

In some aspects, the effector domain comprises a fusion between a polypeptide comprising an amino acid sequence with at least 80% sequence identity to SEQ ID NO:32 or a functional portion thereof and a polypeptide comprising an amino acid sequence with at least 80% sequence identity to SEQ ID NO:33 or a functional portion thereof. In some aspects, the effector domain comprises a fusion between a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:32 or a functional portion thereof and a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:33 or a functional portion thereof. In some aspects, the polypeptide comprising the amino acid sequence of SEQ ID NO: 33 is amino-proximal to the polypeptide of SEQ ID NO:32. In some aspects, the effector domain comprises a fusion between a polypeptide having the amino acid sequence of SEQ ID NO:32 and a polypeptide having the amino acid sequence of SEQ ID NO:33. In some aspects, the effector domain comprises, from amino-proximal to carboxy-proximal end, a polypeptide having the amino acid sequence of of SEQ ID NO:32, a linker, and a polypeptide having the amino acid sequence of SEQ ID NO:33.

In some aspects, the effector domain comprises a fusion between a polypeptide comprising an amino acid sequence that has at least 80% sequence identity to SEQ ID NO:34 or a functional portion thereof and a polypeptide comprising an amino acid sequence that has at least 80% sequence identity to SEQ ID NO:33 or a functional portion thereof. In some aspects, the effector domain comprises a fusion between a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:34 or a functional portion thereof and a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:33 or a functional portion thereof. The polypeptide comprising the amino acid sequence of SEQ ID NO: 34 may be amino-proximal to the polypeptide comprising the amino acid sequence of SEQ ID NO: 33. In some aspects, the polypeptide comprising the amino acid sequence of SEQ ID NO: 33 is amino-proximal to the polypeptide comprising the amino acid sequence of SEQ ID NO: 34. In some aspects, the effector domain comprises a fusion between a polypeptide having the amino acid sequence of SEQ ID NO:34 and a polypeptide having the amino acid sequence of SEQ ID NO:33. In some aspects, the effector domain comprises, from amino-proximal to carboxy-proximal end, a polypeptide having the amino acid sequence of SEQ ID NO:34, a linker, and a polypeptide having the amino acid sequence of SEQ ID NO:33.

In some aspects, the effector domain comprises a fusion between a polypeptide comprising an amino acid sequence that has at least 80% sequence identity to SEQ ID NO:34 or a functional portion thereof and a polypeptide comprising an amino acid sequence that has at least 80% sequence identity to SEQ ID NO:32 or a functional portion thereof. In some aspects, the effector domain comprises a fusion between a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:34 or a functional portion thereof and a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:32 or a functional portion thereof. The polypeptide comprising the amino acid sequence of SEQ ID NO: 34 may be amino-proximal to the polypeptide comprising the amino acid sequence of SEQ ID NO: 32. In some aspects, the polypeptide comprising the amino acid sequence of SEQ ID NO: 32 is amino-proximal to the polypeptide comprising the amino acid sequence of SEQ ID NO: 34. In some aspects, the effector domain comprises a fusion between a polypeptide having the amino acid sequence of SEQ ID NO:34 and a polypeptide having the amino acid sequence of SEQ ID NO:32. In some aspects, the effector domain comprises, from amino-proximal to carboxy-proximal end, a polypeptide having the amino acid sequence of SEQ ID NO:34, a linker, and a polypeptide having the amino acid sequence of SEQ ID NO:32.

In some aspects, the effector domain comprises a fusion between a polypeptide comprising an amino acid sequence that has at least 80% sequence identity to SEQ ID NO:35 or a functional portion thereof and a polypeptide comprising an amino acid sequence that has at least 80% sequence identity to SEQ ID NO:36 or a functional portion thereof. In some aspects, the effector domain comprises a fusion between a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:35 or a functional portion thereof and a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:36 or a functional portion thereof. The polypeptide comprising the amino acid sequence of SEQ ID NO: 35 may be amino-proximal to the polypeptide comprising the amino acid sequence of SEQ ID NO: 36. In some aspects, the polypeptide comprising the amino acid sequence of SEQ ID NO: 36 is amino-proximal to the polypeptide comprising the amino acid sequence of SEQ ID NO: 35. In some aspects, the effector domain comprises a fusion between a polypeptide having the amino acid sequence of SEQ ID NO:35 and a polypeptide having the amino acid sequence of SEQ ID NO:36. In some aspects, the effector domain comprises, from amino-proximal to carboxy-proximal end, a polypeptide having the amino acid sequence of SEQ ID NO:35, a linker, and a polypeptide having the amino acid sequence of SEQ ID NO:36.

In some aspects, the effector domain comprises a fusion between a polypeptide comprising an amino acid sequence that has at least 80% sequence identity to SEQ ID NO:36 or a functional portion thereof and a polypeptide comprising an amino acid sequence that has at least 80% sequence identity to SEQ ID NO:37 or a functional portion thereof. In some aspects, the effector domain comprises a fusion between a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:36 or a functional portion thereof and a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:37 or a functional portion thereof. The polypeptide comprising the amino acid sequence of SEQ ID NO: 36 may be amino-proximal to the polypeptide comprising the amino acid sequence of SEQ ID NO: 37. In some aspects, the polypeptide comprising the amino acid sequence of SEQ ID NO: 37 is amino-proximal to the polypeptide comprising the amino acid sequence of SEQ ID NO: 36. In some aspects, the effector domain comprises a fusion between a polypeptide having the amino acid sequence of SEQ ID NO:36 and a polypeptide having the amino acid sequence of SEQ ID NO:37. In some aspects, the effector domain comprises, from amino-proximal to carboxy-proximal end, a polypeptide having the amino acid sequence of SEQ ID NO:36, a linker, and a polypeptide having the amino acid sequence of SEQ ID NO:37.

In some aspects, the effector domain comprises a fusion between a polypeptide comprising an amino acid sequence that has at least 80% sequence identity to SEQ ID NO:38 or a functional portion thereof and a polypeptide comprising an amino acid sequence that has at least 80% sequence identity to SEQ ID NO:39 or a functional portion thereof. In some aspects, the effector domain comprises a fusion between a polypeptide that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:38 or a functional portion thereof and a polypeptide that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:39 or a functional portion thereof. The polypeptide comprising the amino acid sequence of SEQ ID NO:38 may be amino-proximal to the polypeptide comprising the amino acid sequence of SEQ ID NO:39. In some aspects, the polypeptide comprising the amino acid sequence of SEQ ID NO:39 is amino-proximal to the polypeptide comprising the amino acid sequence of SEQ ID NO:38. In some aspects, the effector domain comprises a fusion between a polypeptide having the amino acid sequence of SEQ ID NO:38 and a polypeptide having the amino acid sequence of SEQ ID NO:39. In some aspects, the effector domain comprises, from amino-proximal to carboxy-proximal end, a polypeptide having the amino acid sequence of SEQ ID NO:38, a linker, and a polypeptide having the amino acid sequence of SEQ ID NO:39.

In some aspects, the effector domain comprises a fusion between a polypeptide comprising an amino acid sequence that has at least 80% sequence identity to SEQ ID NO:69 or a functional portion thereof and a polypeptide comprising an amino acid sequence that has at least 80% sequence identity to SEQ ID NO:70 or a functional portion thereof. In some aspects, the effector domain comprises a fusion between a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:69 or a functional portion thereof and a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:70 or a functional portion thereof. The polypeptide comprising the amino acid sequence of SEQ ID NO: 69 may be amino-proximal to the polypeptide comprising the amino acid sequence of SEQ ID NO: 70. In some aspects, the polypeptide comprising the amino acid sequence of SEQ ID NO: 70 is amino-proximal to the polypeptide comprising the amino acid sequence of SEQ ID NO: 69. In some aspects, the effector domain comprises a fusion between a polypeptide comprising the amino acid sequence of SEQ ID NO:69 and a polypeptide comprising the amino acid sequence of SEQ ID NO:70. In some aspects, the effector domain comprises, from amino-proximal to carboxy-proximal end, a polypeptide comprising the amino acid sequence of SEQ ID NO: 69, a linker, and a polypeptide comprising the amino acid sequence of SEQ ID NO:70.

In some aspects, the effector domain comprises a fusion between a polypeptide comprising an amino acid sequence that has at least 80% sequence identity to SEQ ID NO:69 or a functional portion thereof and a polypeptide comprising an amino acid sequence that has at least 80% sequence identity to SEQ ID NO:71 or a functional portion thereof. In some aspects, the effector domain comprises a fusion between a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:69 or a functional portion thereof and a polypeptide comprising an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:71 or a functional portion thereof. The polypeptide comprising the amino acid sequence of SEQ ID NO: 69 may be amino-proximal to the polypeptide comprising the amino acid sequence of SEQ ID NO: 71. In some aspects, the polypeptide comprising the amino acid sequence of SEQ ID NO: 71 is amino-proximal to the polypeptide comprising the amino acid sequence of SEQ ID NO: 69. In some aspects, the effector domain comprises a fusion between a polypeptide comprising the amino acid sequence of SEQ ID NO:69 and a polypeptide comprising the amino acid sequence of SEQ ID NO:71. In some aspects, the effector domain comprises, from amino-proximal to carboxy-proximal end, a polypeptide comprising the amino acid sequence of SEQ ID NO: 69, a linker, and a polypeptide comprising the amino acid sequence of SEQ ID NO:71.

In some aspects, the effector domain comprises a polypeptide with the amino acid sequence of one of SEQ ID NOs: 19-21, 25-27, 72, or 73, or a polypeptide with an amino acid sequence having at least 80% sequence identity to one of SEQ ID NOs: 19-21, 25-27, 72, or 73, or a functional portion thereof. The effector domain may also comprise a polypeptide with an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOs: 19-21, 25-27, 72, or 73 or a functional portion thereof.

The polypeptides of the disclosure may comprise or further comprise one or more of a stabilizer domain, linker, RNA hairpin binding domain, nuclear export signal, and molecular tag. In some aspects, the polypeptide comprises one or more RNA hairpin binding domain(s). In some aspects, the polypeptide comprises, comprises at least, or comprises at most 1, 2, 3, 4, 5, or 6 RNA hairpin binding domains. The RNA hairpin binding domain may comprise the amino acid sequence of SEQ ID NO:16 or a polypeptide with at least 80% sequence identity to a polypeptide having the amino acid sequence of SEQ ID NO:16 or a functional portion thereof. In some aspects, the RNA hairpin binding domain comprises an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:16 or a functional portion thereof. The polypeptides of the disclosure may comprise or further comprise one or more stabilizer domains. The polypeptides may comprise, comprise at least, or comprise at most 1, 2, 3, 4, 5, or 6 RNA stabilizer domains. The stabilizer domain may comprise the amino acid sequence of SEQ ID NO: 14, 58, or 59 or a polypeptide having an amino acid sequence with at least 80% sequence identity to SEQ ID NO:14, 58, or 59 or a functional portion thereof. The stabilizer domain may comprise SEQ ID NO:14. The stabilizer domain may comprise SEQ ID NO:58 or 59 or a polypeptide having an amino acid sequence with at least 80% sequence identity to SEQ ID NO: 58 or 59 or a functional portion thereof. In some aspects, the stabilizer domain comprises an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:14 or a functional portion thereof. In some aspects, the stabilizer domain may comprises an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:58 or 59 or a functional portion thereof. The polypeptides of the disclosure may comprise or further comprise one or more nuclear export signals (NESs). The polypeptides may comprise, comprise at least, or comprise at most 1, 2, 3, 4, 5, or 6 NESs. The NES may comprise the amino acid sequence of SEQ ID NO:17 or a polypeptide having an amino acid sequence with at least 80% sequence identity to SEQ ID NO:17 or a functional portion thereof. In some aspects, the NES comprises an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:17 or a functional portion thereof. The polypeptides of the disclosure may comprise or further comprise one or more molecular tags. The polypeptides may comprise, comprise at least, or comprise at most 1, 2, 3, 4, 5, or 6 molecular tags. The molecular tag may comprise the amino acid sequence of SEQ ID NO:18 or a polypeptide comprising an amino acid sequence with at least 80% sequence identity to SEQ ID NO:18 or a functional portion thereof. In some aspects, the NES comprises an amino acid sequence that has, has at least, or has at most 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:18 or a functional portion thereof. In some aspects, the molecular tag comprises a FLAG tag.

In aspects of the disclosure, the hairpin binding domain is amino-proximal to the effector domain. In aspects of the disclosure, the stabilizer domain is amino-proximal to the RNA hairpin binding domain and/or effector domain. In aspects of the disclosure, the nuclear export signal is amino-proximal to the effector domain. In aspects of the disclosure, the molecular tag is carboxy-proximal to the effector domain. In aspects of the disclosure, the hairpin binding domain is carboxy-proximal to the effector domain. In aspects of the disclosure, the stabilizer domain is carboxy-proximal to the RNA hairpin binding domain and/or effector domain. In aspects of the disclosure, the nuclear export signal is carboxy-proximal to the effector domain. In aspects of the disclosure, the molecular tag is amino-proximal to the effector domain. In some aspects, the polypeptide comprises, from N-terminus to the C-terminus, a stabilizer domain, a glycine serine linker, a RNA hairpin binding region, a nuclear export signal, an effector domain, and a molecular tag. In some aspects, the polypeptide comprises the amino acid sequence of one of SEQ ID NOs: 1-13, 63-68, 77-82, or 148-169, or an amino acid with at least 80% sequence identity to one of SEQ ID NOs: 1-13, 63-68, 77-82, or 148-169 or a functional portion thereof. The polypeptide may comprise an amino acid sequence that has, has at least, or has at most 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOs: 1-13, 63-68, 77-82, or 148-169 or a functional portion thereof. In some aspects, the polypeptide comprises an amino acid sequence that has at least one substitution relative to SEQ ID NOs: 1-13, 63-68, 77-82, or 148-169. In some aspects, the polypeptide has an amino acid sequence that comprises, comprises at least, or comprises at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 substitutions relative one of SEQ ID NOs: 1-13, 63-68, 77-82, or 148-169.

The delivery vehicles of the disclosure may comprise liposome(s), particle(s), exosome(s), microvesicle(s), a gene-gun or one or more nucleic acid vector(s). In some aspects, the delivery vehicle or composition of the disclosure comprises or further comprises a RNA targeting molecule comprising a RNA targeting region and at least one hairpin structure, wherein the hairpin structure of the RNA targeting molecule specifically binds to the RNA hairpin binding domain of the polypeptide. In some aspects, the RNA targeting molecule comprises a TAR hairpin scaffold or a human SLBP hairpin scaffold. In some aspects, the RNA targeting molecule comprises SEQ ID NO:40 or 41. The RNA targeting region may hybridizes to the 5′UTR of a RNA. The RNA targeting region may hybridize to the 3′UTR of a RNA. The RNA targeting region may hybridize to the 5′UTR or 3′UTR of a mammalian mRNA. In some aspects, the RNA targeting region comprises a nucleic acid sequence of one of SEQ ID NOs: 55, 83-86, 88-112, 122-131, or 144-147 or a nucleic acid sequence having at least 80% sequence identity to one of SEQ ID NOs: 55, 83-86, 88-112, 122-131, or 144-147. The RNA targeting region may comprise a nucleic acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity (or any derivable range therein) to one of SEQ ID NOs: 55, 83-86, 88-112, 122-131, or 144-147. The RNA targeting molecule may comprise at least one of SEQ ID NO:40-54, 56, 57, or the sequence UCCCGA. In some aspects, the RNA targeting molecule comprises, comprises at least, or comprises at most 1, 2, 3, 4, 5, 6, 7, or 8 hairpin structures (or any derivable range therein), wherein each hairpin structure is independently chosen from one of one of SEQ ID NO:40-54, 56, 57, or the sequence UCCCGA.

Modulating at least one target RNA may comprise cleaving, demethylating, methylating, activating translation, repressing translation, promoting degradation, or binding to the RNA. Modulating at least one target RNA may exclude cleaving, demethylating, methylating, activating translation, repressing translation, promoting degradation, or binding to the RNA. In some aspects, at least two target RNAs are modulated. In some aspects, at least 2, 3, 4, 5, 6, 7, 8, 9, 10, 15, 20, or 25 (or any derivable range therein) target RNAs are modulated. The multiple RNAs may be modulated by the same effector domain or by an effector domain with the same activity. In some aspects, the different target RNAs are modulated with a different activity, such as by cleaving, demethylating, methylating, activating translation, repressing translation, promoting degradation, or binding to the RNA. The effector domain may be one that does not bind to RNA. In some aspects, the effector domain comprises a polypeptide that does not have RNA binding activity. In some aspects, the effector domain does not bind to modified RNA. In some aspects, the effector domain does not bind to m6A modified RNA.

A stabilizer domain, a glycine serine linker, a RNA hairpin binding region, a nuclear export signal, an effector domain, a molecular tag, a hairpin structure, and/or RNA targeting region may exclude one or more of SEQ ID NOs: 1-169.

The term “RNA hairpin” refers to a RNA molecule with stem-loop intramolecular base pairing. A hairpin can occur when two regions of the same strand, usually complementary in nucleotide sequence when read in opposite directions, base-pair to form a double helix that ends in an unpaired loop. The disclosure relates to engineered RNA targeting molecules comprising a RNA targeting region and one or more hairpins. Accordingly, the engineered RNA molecules of the disclosure are chimeric molecules that are non-naturally occurring.

The term “RNA targeting region” refers to a region of the RNA that is capable of hybridizing to a target RNA. The target RNA may be a disease associated RNA or one that is a modulation target according to the current systems and methods.

The “effector domain” refers to a peptide or polypeptide that has activity directed to RNA. Examples of activity include methylation activity, RNA-binding activity, nuclease activity, and translational activation or repression activity.

In some aspects, the RNA hairpin binding domain and the effector domain are operably linked. The term “operably linked” refers to two proteins that are linked through either covalent or non-covalent interactions. For example, the two proteins may be covalently linked through a peptide bond. In some aspects, the proteins are non-covalently linked. One or more proteins of the disclosure may be operably linked to another protein through linkage to a pair of accessory proteins that have a strong affinity for each other. Such accessory proteins are known in the art. For example, the SunTag is one such system that includes an antibody with a strong affinity for a peptide. One protein, polypeptide, or domain of the disclosure may be linked to a SunTag peptide and another protein, polypeptide, or domain of the disclosure may be linked to an antibody to allow operable linkage of the two proteins, polypeptides, or domains through the interaction of the SunTag peptide and antibody. Further examples include biotin and avidin/streptavidin and spytag and spycatcher.

In some aspects, the system is inducible by providing the effector domain and the hairpin binding domain as two unlinked polypeptides that become linked upon the presence of a stimulant. The induction may be, for example, by light induction or by chemical induction. Such inducibility allows for activation of the RNA regulation at a desired moment in time. In some aspects, the effector domain is covalently linked to a first dimerization domain and the RNA hairpin binding domain is covalently linked to a second dimerization domain and wherein the first and second dimerization domain are capable of dimerizing to form a non-covalent or covalent linkage. In some aspects, the dimerization is inducible. In some aspects, the dimerization is induced through binding of the dimerization domains to a ligand. The term inducible refers to dimerization that is formed in response to a stimulus, such as a ligand, a chemical, a temperature change, or light, for example.

Light inducibility is for instance achieved by designing a fusion complex wherein the first and second dimerization domains comprise CRY2 PHR and CIBN. This system is particularly useful for light induction of protein interactions in living cells and is further described in Konermann S, et al. Nature. 2013; 500:472-476, which is herein incorporated by reference.

Suitable dimerization domains and corresponding ligands are known in the art. For example, Liang, F. S., Ho, W. Q., and Crabtree, G. R. (2011). Engineering the ABA plant stress pathway for regulation of induced proximity. Sci. Signal. 4, rs2, which is incorporated by reference, describes suitable dimerization/ligand systems that are useful in aspects of the disclosure. In some aspects, one of the first or second dimerization domain comprises PYR/PYR1-like (PYL1), the other of the first or second domain comprises ABA insensitive 1 (ABI1), and the ligand comprises abscisic acid (ABA) or derivatives or fragments thereof. The dimerization domain may be a fragment or portion of the whole protein and may be a substituted or modified. In some aspects, the first and/or second dimerization domain comprises FKBP12 and the ligand comprises FK1012 or derivatives or fragments thereof. In some aspects, one of the first or second dimerization domain comprises FK506 binding protein (FKBP), the other of the first or second domain comprises FKBP-Rap binding domain of mammalian target of Rap mTOR (Frb), and the ligand comprises rapamycin (Rap) or derivatives or fragments thereof.

Derivatives refer to modified ligands and domains that retain binding or have enhanced binding to their dimerization domain or ligand, respectively. Fragments refer to contiguous portions of the dimerization domains that retain binding to the ligand. In some aspects, the dimerization domain may be a modified fragment.

In some aspects, the domains of the disclosure are human or are human-derived. In some aspects, the polypeptide is non-immunogenic. A human protein, polypeptide, domain, or nucleic acid refers to a protein, polypeptide, domain, or nucleic acid that is from the human genome, although it may be produced recombinantly in non-human systems. The term “human-derived” refers to a protein, polypeptide, domain, or nucleic acid that is a variant or fragment of a protein, polypeptide, domain, or nucleic acid from the human genome, although it may be produced recombinantly in non-human systems. In some aspects, the fusion protein, conjugate, system, or parts thereof, such as parts i, ii, and/or iii are non-immunogenic and/or non-toxic when expressed in or administered to humans.

In some aspects, the nucleic acids or polypeptides of the disclosure are synthetic, are non-natural, and/or do not occur naturally in nature. The polypeptides and methods of the disclosure may exclude a Cas9 enzyme or a polypeptide that comprises or comprises at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 amino acids that have or have at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to a Cas9 protein. Nucleic acids and methods of the disclosure exclude nucleic acids that bind to a Cas9 protein.

In some aspects, the RNA targeting molecule comprises exactly one hairpin. In some aspects, the RNA targeting molecule comprises at least one hairpin. In some aspects, the RNA targeting molecule comprises exactly two hairpins. In some aspects, the RNA targeting molecule comprises at least two hairpins. In some aspects, the RNA targeting molecule comprises exactly three hairpins. In some aspects, the RNA targeting molecule comprises at least three hairpins. In some aspects, the RNA targeting molecule comprises exactly four hairpins. In some aspects, the RNA targeting molecule comprises at least four hairpins. In some aspects, the RNA targeting molecule comprises exactly five hairpins. In some aspects, the RNA targeting molecule comprises at least five hairpins. In some aspects, the RNA targeting molecule comprises 1˜4 hairpins. In some aspects, the RNA targeting molecule comprises 1-3 hairpins. In some aspects, the RNA targeting molecule comprises 1-2 hairpins. In some aspects, the RNA targeting molecule comprises 2-4 hairpins. In some aspects, the RNA targeting molecule comprises 2-3 hairpins. In some aspects, the RNA targeting molecule comprises at least, at most, or exactly 1, 2, 3, 4, 5, or 6 hairpins (or any range derivable therein). In some aspects, the RNA targeting molecule comprises at least one hairpin that does not bind to the RNA hairpin binding protein and at least one hairpin that binds to the RNA hairpin binding protein. In some aspects, the RNA targeting molecule binds to more than one RNA binding protein. In some aspects, the RNA targeting molecule comprises two, three, or four hairpin structures and binds to at least two RNA binding proteins. In some aspects, the effector system comprises at least two regulatory domains, wherein each regulatory domain binds to a different RNA binding molecule.

In some aspects, the RNA targeting molecule comprises one or more modified nucleotides. In some aspects, the modified nucleotides comprise a modification such as a phosphorothioate, locked nucleotides, ethylene bridged nucleotides, peptide nucleic acids, 5′E-VP, or is modified to a morpholino. In some aspects, the modification includes one described herein.

In some aspects, the effector domain comprises a nuclease, methylase, demethylase, translational activator, translational repressor, single-stranded RNA cleavage activity, double-stranded RNA cleavage activity, or RNA binding activity. In some aspects, the effector domain comprises an activity described herein.

In some aspects, the effector domain increases translation of a target RNA (e.g., by recruiting the translational machinery). In some aspects, the effector domain increases degradation of a target RNA. In some aspects, the effector domain modifies the localization of a target RNA. In some aspects, the effector domain modifies the processing of the target RNA (e.g., by active nuclease activity on the target or by triggering endogenous epitranscriptomic regulatory pathways).

In some aspects, the vectors of the disclosure may comprise or further comprise a regulatory element operably linked to the nucleotide of the disclosure. Regulatory elements, in addition to a NLS and NES, as previously described, also include promoters, polyadenylation signals, enhancers, etc. Other regulatory elements are known in the art and described herein and may be used in the aspects of the disclosure. In some aspects, the one or more nucleic acid vectors are optimized for expression in an eukaryotic cell. In some aspects, the expression of the domains, RNA, or polypeptides in the cell or from a vector is constitutive. In some aspects, the expression of the domains, RNA, or polypeptides in the cell or from a vector is conditional. In some aspects, the expression vector further comprises a nucleic acid encoding a RNA targeting molecule. In some aspects, one or more of the vectors are viral vectors. In some aspects, the one or more vectors comprise one or more retroviral, lentiviral, adenoviral, adeno-associated or herpes simplex viral vectors. In some aspects, one or more of the vectors are non-viral vectors. In some aspects, the system or composition is non-viral, which denotes that it does not contain any viral components.

In some aspects, there is a system or kit comprising one or more of the following components: a polypeptide comprising an effector domain, a polypeptide comprising a RNA binding domain, a polypeptide comprising a stabilizer, a nucleic acid encoding for a effector domain, a nucleic acid encoding for a RNA binding domain, a nucleic acid encoding a stabilizer, a nucleic acid encoding a RNA targeting molecule comprising a RNA targeting region and at least one hairpin structure; a conjugate of the disclosure; a vector of the disclosure, a fusion protein of the disclosure, a recombinant host cell, an expression construct, an engineered viral vector, or an engineered attenuated virus. In certain aspects, a polypeptide of the disclosure is under the control of a heterologous promoter. It is specifically contemplated that any protein or polypeptide function that are used in aspects, may be used a nucleic acid encoding that protein or polypeptide function. Also, any and all polypeptides, proteins, nucleic acid molecules may be contained within a cell or other living organism, such as a virus (for instance, a phage).

A kit may include one or more components that are separate or together in a suitable container means, such as a sterile, non-reactive container. In some aspects, cells or viruses are provided that contain one or more nucleic acid constructs that encode the polypeptides of the disclosure. The term “promoter” is used according to its ordinary meaning to those in the field of molecular biology; it generally refers to a site on a nucleic acid in which a polymerase can bind to initiate transcription. In specific aspects, the promoter is recognized by a T7 RNA polymerase.

The compositions, vectors, systems, methods, and proteins of the disclosure are useful for a variety of clinical and research-related applications. The aspects of the disclosure may be useful for the treatment of a disease or condition, such as cancer or autoimmunity. In some aspects, the methods and compositions are for the acute treatment of a disease or condition. In some aspects, the methods and compositions are useful for the temporary modulation of RNA. In some aspects exclude permanent modification of gene activity. In some aspects, the methods and compositions are safer due to the acute modulation of RNA and/or due to the ability to control the expression of the system in vivo.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments and aspects are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B: gRNA screen across mPMP22 measuring gRNA-dependent RNA decay by RT-qPCR in NIH/3T3 cells. Significant changes in degradation were observed depending on where the transcript is targeted as well as the effectors and CIRTS localization tag used. (A) CIRTS-YTHDF2 (1-200aa) with nuclear export tag (NES). (B) CIRTS-Pin nuclease with nuclear localization tag (NLS). All guides represent 3 or more biological replicates.

FIG. 2. PMP22 activation. Targeting PMP22 with eIF4E and Y1-based CIRTS activators with 5 gRNAs. g15 (targets 5′UTR) and g17 (targets 3′UTR) activate PMP22 expression, especially with an eIF4E-based CIRTS system.

FIG. 3. p53 RNA editing. FIG. 3 demonstrates that a CIRTS-ADAR editor delivered to cells along with gRNAs can edit p53 mismatch sites and the highest editing is observed when the mismatch occurs 15 nucleotides from the 5′ end of the gRNA sequence. CIRTS-ADAR editor delivered to cells along with a p53 reporter vector. Guide tiling along the mismatch site and monitoring editing efficiency by Sanger sequencing RT-qPCR products. HEK293T cells with CIRTS-ADAR and 2TAR guides with lipofectamine 2000 (P53 R175H reporter (12 ng)+CIRTS-ADAR (125 ng)+guide (100 ng)).

FIG. 4: CIRTS degrader panel targeting the PMP22 transcript in NIH-3T3 cells measured by RT-qPCR. Each CIRTS degrader is normalized to a gRNA-matched, CIRTS-GFP negative control. Data are mean±SEM; n=3 or more biological replicates.

FIG. 5: CIRTS Y2-based degrader can decrease SNCA transcript levels. Values shown as mean±SEM from n=3 biological replicates. Student's t-test: * p<0.05 and ** p<0.01

FIG. 6A-B: Dual luciferase reporter system for assessing translational activators targeting native UTR contexts. A) Dual luciferase reporter expresses two separate luciferase transcripts. Only one (Firefly luciferase, Fluc) will carry the target UTR sequence. B) CIRTS activators directed by gRNA complementary to the target UTR are expected to specifically increase Fluc translation, and not that of Nano luciferase (Nluc).

FIG. 7: CIRTS activators that boost Fluc expression when targeted to the PMP22 3′ UTR and normalized to an effector matched NT gRNA control. Data are mean±SEM; n=3 or more biological replicates.

FIG. 8: CIRTS-Y1 activators can boost protein levels in JAG1 and SNC1a reporter systems. Data are mean±SEM; n=3 biological replicates. Significance determined by Student's t-test.

FIG. 9: CIRTS-Y1 increases SCN1a proteins levels in Neuro 2a cells when directed by a gRNA complimentary to the SCN1a transcript 3′ UTR (g1) compared to a non-targeting gRNA (NT). Band intensity quantification completed in EmpiriaStudio. n=2 biological replicates.

### DETAILED DESCRIPTION

Epitranscriptomic regulation controls information flow through the central dogma and provides unique opportunities for manipulating cell state at the RNA level. However, both fundamental mechanistic studies and potential translational applications are impeded by a lack of effective methods to target specific RNAs with effector proteins. Here, the inventors present the design and validation of a CRISPR/Cas-inspired RNA targeting system (CIRTS), a new strategy for constructing programmable systems. The inventors show that CIRTS is a simple and generalizable approach to deliver a range of effector proteins, including nucleases, degradation machinery, and translational activators, to target transcripts. CIRTS complexes are not only smaller than naturally-occurring CRISPR/Cas programmable RNA binding systems, but can be built entirely from human parts. The small size and human-derived nature of CIRTS provides a less perturbative method for fundamental RNA regulatory studies as well as a potential strategy to avoid immune issues when applied to epitranscriptome-modulating therapies.

### I. RNA Effector Domains

The RNA effector domain may be a polypeptide or functional portion thereof of any one of SEQ ID NOs: 19-39, 60-62, 69-76, 113-121, 136-143 and combinations and fusion proteins thereof. RNA effector domains are described herein. The Table below exemplifies functions of the RNA effector domain.

| Description | Exemplary Function | Seq ID |
|---|---|---|
| ZFP36 | ZFP36 (ZFP36 Ring Finger Protein) is a Zinc-finger RNA-binding | 32 |
| | protein that destabilizes several cytoplasmic AU-rich element (ARE)- | |
| | containing mRNA transcripts by promoting their poly(A) tail removal | |
| | or deadenylation, and hence providing a mechanism for attenuating | |
| | protein synthesis | |
| YTHDF2 | YTH N6-Methyladenosine RNA Binding Protein 2 (YTHDF2) | 33 |
| | specifically recognizes and binds N6-methyladenosine | |
| CNOT7 | CCR4-NOT Transcription Complex Subunit 7 (CNOT7) has 3′-5′ | 34 |
| | poly(A) exoribonuclease activity for poly(A) RNA substrate. | |
| GCN2 | General control nonderepressible 2 (GCN2) phosphorylates eIF2α | 22 |
| | causing it to function as a competitive inhibitor of eIF2B and therefore | |
| | decreasing translation. | |
| 4EBP1 | Eukaryotic translation initiation factor 4E-binding protein 1 (4EBP1) is | 23 |
| | a repressor of translation initiation that regulates EIF4E activity by | |
| | preventing its assembly into the elF4F complex: hypophosphorylated | |
| | form competes with EIF4G1/EIF4G3 and strongly binds to EIF4E, | |
| | leading to repress translation. | |
| NANOS3 | NANOS3 is a zinc-finger RNA-binding protein that interacts with the | 24 |
| | ubiquitously expressed protein Pumilio. The NANOS3-Pumilio | |
| | complex binds to the 3′ UTR of target mRNA and represses | |
| | translation. | |
| EIF4E | Eukaryotic translation initiation factor 4E (eIF4E) recognizes and binds | 35 |
| | the 7-methylguanosine-containing mRNA cap during an early step in | |
| | the initiation of protein synthesis and facilitates ribosome binding by | |
| | inducing the unwinding of the mRNAs secondary structures | |
| EIF4A | Eukaryotic initiation factor 4A-I (eIF4A-I) is an ATP-dependent RNA | 36 |
| | helicase which is a subunit of the eIF4F complex involved in cap | |
| | recognition and is required for mRNA binding to ribosome. | |
| PAIP | Polyadenylate-binding protein-interacting protein 1 (PAIP) acts as a | 37 |
| | coactivator in the regulation of translation initiation of poly(A)- | |
| | containing mRNAs. Its stimulatory activity on translation can be | |
| | mediated via its action on PABPC1. | |
| YTHDF1 | Specifically recognizes and binds N6-methyladenosine (m6A)- | 38 |
| | containing mRNAs, and promotes mRNA translation efficiency. | |
| YTHDF3 | Specifically recognizes and binds N6-methyladenosine (m6A)- | 39 |
| | containing RNAs and promotes RNA translation efficiency. | |
| EIF2S2 | Functions in the early steps of protein synthesis by forming a ternary | 28 |
| | complex with GTP and initiator tRNA. This complex binds to a 40S | |
| | ribosomal subunit, followed by mRNA binding to form a 43S | |
| | preinitiation complex. Junction of the 60S ribosomal subunit to form | |
| | the 80S initiation complex is preceded by hydrolysis of the GTP bound | |
| | to eIF-2 and release of an eIF-2-GDP binary complex. | |
| HSPB1 | Heat shock protein B1 has been demonstrated to bind eIF4E and | 29 |
| | repress translation, but it has also been identified in screens to | |
| | positively regulation mRNA stability and translation. | |
| YWHAE | YWHAE is a member of the 14-3-3 protein family that has been | 30 |
| | implicated in various regulatory processes and has been identified in | |
| | screens to positively regulation mRNA stability and translation. | |
| SF3B3 | Involved in pre-mRNA splicing as a component of the splicing factor | 31 |
| | SF3B complex, a constituent of the spliceosome. SF3B complex is | |
| | required for “A” complex assembly formed by the stable binding of U2 | |
| | snRNP to the branchpoint sequence (BPS) in pre-mRNA. | |
| SNRPA | Component of the spliceosomal U1 snRNP, which is essential for | 60 |
| | recognition of the pre-mRNA 5′ splice-site and the subsequent | |
| | assembly of the spliceosome. U1 snRNP is the first snRNP to interact | |
| | with pre-mRNA. | |
| TNRC6B | Trinucleotide repeat-containing gene 6B (TNRC6B) plays a role in | 61 |
| | RNA-mediated transcription degradation and can recruit deadenylase | |
| | complexes to destabilize target mRNA. | |
| DAZ4 | Deleted in azoospermia protein 4 (DAZ4) is an RNA-binding protein | 62 |
| | that binds to the 3′ UTR of target mRNA and increases their translation | |
| | by interacting with PABP. | |
| YB-1 | YB-1 is a DNA- and RNA-binding protein involved in various | 69 |
| | processes, such as translational repression, RNA stabilization, mRNA | |
| | splicing, DNA repair and transcription regulation. YB-1 can act as a | |
| | RNA-binding protein: binds preferentially to the 5′-[CU]CUGCG-3′ | |
| | RNA motif and can specifically recognizes mRNA transcripts modified | |
| | by C5-methylcytosine (m5C). YB-1 can promote mRNA stabilization: | |
| | acts by binding to m5C-containing mRNAs and recruiting the mRNA | |
| | stability maintainer ELAVL1, thereby preventing mRNA decay. YB-1 | |
| | can contribute to the regulation of translation by modulating the | |
| | interaction between the mRNA and eukaryotic initiation factors (By | |
| | similarity). YB-1 can play a role in RNA composition of extracellular | |
| | exosomes by defining the sorting of small non-coding RNAs, such as | |
| | tRNAs, Y RNAs, Vault RNAs and miRNAs. YB-1 may sort RNAs in | |
| | exosomes by recognizing and binding C5-methylcytosine (m5C)- | |
| | containing RNAs. YB-1 is also involved in pre-mRNA alternative | |
| | splicing regulation: binds to splice sites in pre-mRNA and regulates | |
| | splice site selection. | |
| ADAR2 | ADAR2 (E488Q) features a point mutation compared to ADAR2 that | 70 |
| (E488Q) | increases its base editing catalytic rate. | |
| ADAR2 | Catalyzes the hydrolytic deamination of adenosine to inosine in | 71 |
| (wt) | double-stranded RNA (dsRNA) referred to as A-to-I RNA editing. | |
| FXR1 | Fragile X-related-1 (FXR1) binds to AU-rich elements and stabilizes | 74 |
| | the expression of target mRNA. FXR1 has also been demonstrated to | |
| | interact with eIF4A and eIF4E. | |
| APPLE | APPLE is a micropeptide that is encoded by a non-coding RNA | 75 |
| | transcript that promotes translation by facilitating the PABP-eIF4G | |
| | interaction. | |
| eIF4E2 | Similar to eIF4E, eIF4E2 also binds the 7-methylguanosine-containing | 76 |
| | mRNA cap, but unlike eIF4E, cannot recruit eIF4G. Therefore, eIF4E2 | |
| | inhibits translation by preventing eIF4F complex formation and | |
| | translation initiation. | |

### II. RNA Hairpin Binding Domains and Hairpin Structures

Various RNA hairpin binding domains and hairpin structures that they bind are known in the art and can be used in the systems, compositions, fusion proteins, kits, vectors, and methods of the disclosure. For example, aspects include a RNA hairpin binding domain and hairpin structure according to the following table (Table 1), which lists proteins comprising RNA hairpin binding domains and the hairpin structure to which they specifically bind”

TABLE 1
RNA hairpin binding domain

| RNA hairpin | |
|---|---|
| binding domain | Hairpin structure |
| MS2 coat protein | AAACAUGAGGAUUACCCAUGU (SEQ ID NO: 42) |
| (MCP) | AAACAUGAGGAUCACCCAUGU (SEQ ID NO: 43) |
| (bacteriophage) | |
| λ N | GCCCUGAAGAAGGGC (SEQ ID NO: 44); |
| (bacteriophage) | GCCCUGAAAAAGGGC (SEQ ID NO: 45) |
| PP7 | UAAGGAGUUUAUAUGGAAACCCUUA (SEQ ID NO: 46) |
| (Pseudomonas | |
| aeroginosa) | |
| Qβ | AAACAUGAGGAUUACCCAUGU (SEQ ID NO: 47); |
| | AAACAUGAGGAUCACCCAUGU (SEQ ID NO: 48); |
| | AUGCAUGUCUAAGACAGCAU (SEQ ID NO: 49) |
| GA | AAACAUGAGGAUUACCCAUGU (SEQ ID NO: 50); |
| | AAACAUGAGGAUCACCCAUGU (SEQ ID NO: 51); |
| | AAAACAUAAGGAAAACCUAUGUU (SEQ ID NO: 52) |
| Bovine | GGCUCGUGUAGCUCAUUAGCUCCGAGCC (SEQ ID NO: 53) |
| immunodeficiency | |
| virus (BIV) | |
| transactivator of | |
| transcription (tat) | |
| KU70 (HUMAN) | GGCGUCCCUCCCGAAGCUGCGCGCUCGGUCGAACAGGACG |
| | ACC (SEQ ID NO: 54) |
| NUCLEOLIN | UCCCGA; |
| (HUMAN) | GGCCGAAAUCCCGAAUGAGGCC (SEQ ID NO: 56); |
| | GGAUGCCUCCCGAGUGCAUCC (SEQ ID NO: 57). |

It is contemplated that multiple RNA hairpin binding domains and/or the Effector domain may be used in a multiplexed fashion by using RNA hairpin binding domains that bind to different hairpin structures to target multiple different RNAs in the same cell. Different targeted RNAs may be modulated in the same or in different ways. For example, one RNA may be modulation with translational activation, while a second RNA may be modulated with translational repression in the same cell. Therefore, the systems of the disclosure can be used in a multiplexed fashion for the modulation of at least 2, 3, 4, 5, 6, 7, 8, 9, 10 or more RNAs in one cell, tissue, or organisms.

### III. Stabilizer Domain

Exemplary stabilizer domains include those in the table below as well as variants of those represented by the amino acid sequence below.

| Description | Amino Acid Sequence |
|---|---|
| ORF5 | MDDPSFLTGRSTYAKRRRARRMNVCKCGAILHNNKDCRSSTISGHKL |
| | DRLRFVKEGRVALEGETPVYRTWVKWVETEYHINILETSDDEE (SEQ |
| | ID NO: 58) |
| HBEGF | MRVTLSSKPQALATPNKEEHGKRKKKGKGLGKKRDPCLRKYKDFCI |
| | HGECKYVKELRAPSCICHPGYHGERCHGLS (SEQ ID NO: 59) |
| β-defensin | MGIINTLQKYYCRVRGGRCAVLSCLPKEEQIGKCSTRGRKCCRRKK |
| | (SEQ ID NO: 14) |

Stabilizer proteins are proteins that can bind to the gRNA sequence non-specifically, in some embodiments via charge-based interactions, and can be used to stabilize and protect the guiding RNA prior to target engagement.

### IV. Nucleic Acids

In certain aspects, there are recombinant nucleic acids encoding the proteins, polypeptides, regulatory domains, or RNA targeting molecules described herein.

As used in this application, the term “polynucleotide” refers to a nucleic acid molecule that either is recombinant or has been isolated free of total genomic nucleic acid. Included within the term “polynucleotide” are oligonucleotides (nucleic acids 100 residues or fewer in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein (see above).

In particular aspects, there are isolated nucleic acid segments and recombinant vectors incorporating nucleic acid sequences that encode a polypeptides (e.g., a polymerase, RNA polymerase, one or more truncated polymerase domains or interaction components that are polypeptides) that drive gene transcription dependent on polymerase activity from the polymerase domains when the interaction components interact. The term “recombinant” may be used in conjunction with a polypeptide or the name of a specific polypeptide, and this generally refers to a polypeptide produced from a nucleic acid molecule that has been manipulated in vitro or that is a replication product of such a molecule . . .

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

### A. Vectors

Polypeptides may be encoded by a nucleic acid molecule. The nucleic acid molecule can be in the form of a nucleic acid vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Vectors may be used in a host cell to produce a polymerase, RNA polymerase, one or more truncated polymerase domains or interaction components that are fused, attached or linked to the one or more truncated RNA polymerase domains.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

### B. Cells

The disclosure provides methods for modifying a target RNA of interest, in particular in prokaryotic cells, eukaryotic cells, tissues, organs, or organisms, more in particular in mammalian cells, tissues, organs, or organisms. The target RNA may be comprised in a nucleic acid molecule within a cell. In some aspects, the target RNA is in a eukaryotic cell, such as a mammalian cell or a plant cell. The mammalian cell many be a human, non-human primate, bovine, porcine, rodent or mouse cell. The cell may be a non-mammalian eukaryotic cell such as poultry, fish or shrimp. The plant cell may be of a crop plant such as cassava, corn, sorghum, wheat, or rice. The plant cell may also be of an algae, tree or vegetable. The modulation of the RNA induced in the cell by the methods, systems, and compositions of the disclosure may be such that the cell and progeny of the cell are altered for improved production of biologic products such as an antibody, starch, alcohol or other desired cellular output. The modulation of the RNA induced in the cell may be such that the cell and progeny of the cell include an alteration that changes the biologic product produced.

The mammalian cell may be a human or non-human mammal, e.g., primate, bovine, ovine, porcine, canine, rodent, Leporidae such as monkey, cow, sheep, pig, dog, rabbit, rat or mouse cell. The cell may be a non-mammalian eukaryotic cell such as poultry bird (e.g., chicken), vertebrate fish (e.g., salmon) or shellfish (e.g., oyster, clam, lobster, shrimp) cell. The cell may also be a plant cell. The plant cell may be of a monocot or dicot or of a crop or grain plant such as cassava, corn, sorghum, soybean, wheat, oat or rice. The plant cell may also be of an algae, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.).

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

### C. Expression Systems

Numerous expression systems exist that comprise at least a part or all of the compositions discussed above. Prokaryote- and/or eukaryote-based systems can be employed for use with an aspect to produce nucleic acid sequences, or their cognate polypeptides, proteins and peptides. For example, the vectors, fusion proteins, RNA hairpin binding proteins, RNA targeting molecules, Effector domain, and accessory proteins of the disclosure may utilize an expression system, such as an inducible or constitutive expression system. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems, other examples of expression systems include STRATAGENER's COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### D. Conjugation of Nucleic Acids and Polypeptides

Aspects of the disclosure relate to the conjugation of nucleic acids to polypeptides. Methods of conjugation of nucleic acids to polypeptides are known in the art and include those described below. Aspects of the disclosure relate to methods of making nucleic acid-polypeptide molecules and the molecules themselves wherein the nucleic acid has been conjugated to the polypeptide by way of a method described herein. One such example includes click chemistry. The “click reaction”, also known as “click chemistry” is a name often used to describe a stepwise variant of the Huisgen 1,3-dipolar cycloaddition of azides and alkynes to yield 1,2,3-triazole. This reaction is carried out under ambient conditions, or under mild microwave irradiation, typically in the presence of a Cu(I) catalyst, and with exclusive regioselectivity for the 1,4-disubstituted triazole product when mediated by catalytic amounts of Cu(I) salts [V. Rostovtsev, L. G. Green, V. V. Fokin, K. B. Sharpless, Angew. Chem. Int. Ed. 2002, 41, 2596; H. C. Kolb, M. Finn, K. B. Sharpless, Angew Chem., Int. Ed. 2001, 40, 2004].

In other conjugation methods, a mutant form of the human DNA repair protein 06-alkylguanine-DNA alkyltransferase reacts rapidly and specifically with 06-benzylguanin (BG) and also with derivatives that carry a large moiety linked to the benzyl group. With guanine as the leaving group, the benzyl moiety becomes covalently attached to a cysteine in the active site of the enzyme. The enzyme has also been mutagenized to become specific for 06-benzylcytosine (BC) in a similar manner. These enzyme domains (about 20 kDa) are commercially available as SNAP and CLIP tags, respectively.

A further conjugation method utilizes the Halo tag. The Halo tag makes use of a chemical reaction orthogonal to eukaryotes, i.e. the dehalogenation of haloalkane ligands, thus, leading to highly specific covalent labelling of the tag, and therefore protein, in both live and fixed cells.

### E. Nucleic Acid Modifications

The oligonucleotides of the disclosure, such as the RNA targeting molecules and other nucleic acids described herein may have modifications, for example that increase the stability of the nucleic acid. In some aspects, the RNA targeting molecule is an oligonucleotide analog. The term “oligonucleotide analog” refers to compounds which function like oligonucleotides but which have non-naturally occurring portions (e.g., that include one or more non-natural residues or linkages). Oligonucleotide analogs can have altered sugar moieties, altered base moieties or altered inter-sugar linkages. The term “oligomers” is intended to encompass oligonucleotides, oligonucleotide analogs or oligonucleosides. Thus, in speaking of “oligomers” reference is made to a series of nucleosides or nucleoside analogs that are joined via either natural phosphodiester bonds or other linkages, including the four atom linkers. Although the linkage generally is from the 3′ carbon of one nucleoside to the 5′ carbon of a second nucleoside, the term “oligomer” can also include other linkages such as 2′-5′ linkages.

Oligonucleotide analogs also can include other modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true nucleic acid species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

The present disclosure concerns modified oligonucleotides, i.e., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound. Preferred linkages have structure CH2--RA--NR1 CH2, CH2--NR1--RA--CH2, RA--NR1--CH2--CH2, CH2--CH2--NR1--RA, CH2--CH2--RA--NR1, or NR1--RA--CH2--CH2 where RA is O or NR2.

Modifications to the nucleic acid molecules of the disclosure may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon is modified at its terminal 3′ site, while a “downstream” synthon is modified at its terminal 5′ site.

Oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass [53], TentaGel Support—an aminopolyethyleneglycol derivatized support or Poros—a copolymer of polystyrene/divinylbenzene. Attachment and cleavage of nucleotides and oligonucleotides can be effected via standard procedures [55]. As used herein, the term solid support further includes any linkers (e.g., long chain alkyl amines and succinyl residues) used to bind a growing oligonucleoside to a stationary phase such as CPG.

### 1. Locked Nucleotides

In some aspects, the nucleic acid of the disclosure, such as the RNA targeting molecule comprises a locked nucleic acid. A locked nucleic acid (LNA or Ln), also referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. Such oligomers are synthesized chemically and are commercially available. The locked ribose conformation enhances base stacking and backbone pre-organization. This significantly increases the hybridization properties (melting temperature) of oligonucleotides.

### 2. Ethylene Bridged Nucleotides

In some aspects, the nucleic acid of the disclosure, such as the RNA targeting molecule comprises one or more ethylene bridged nucleotides. Ethylene-bridged nucleic acids (ENA or En) are modified nucleotides with a 2′-0, 4′C ethylene linkage. Like locked nucleotides, these nucleotides also restrict the sugar puckering to the N-conformation of RNA.

### 3. Peptide Nucleic Acids

In some aspects, the nucleic acid of the disclosure, such as the RNA targeting molecule comprises one or more peptide nucleic acids. Peptide nucleic acids (PNA or Pn) mimic the behavior of DNA and binds complementary nucleic acid strands. The term, “peptide,” when used herein may also refer to a peptide nucleic acid. PNA is an artificially synthesized polymer similar to DNA or RNA. DNA and RNA have a deoxyribose and ribose sugar backbone, respectively, whereas PNA's backbone is composed of repeating N-(2-aminoethyl)-glycine units linked by peptide bonds. The various purine and pyrimidine bases are linked to the backbone by a methylene bridge (—CH2—) and a carbonyl group (—(C═O)—). PNAs are depicted like peptides, with the N-terminus at the first (left) position and the C-terminus at the last (right) position.

Since the backbone of PNAs contains no charged phosphate groups, the binding between PNA/DNA strands is stronger than between DNA/DNA strands due to the lack of electrostatic repulsion. PNAs are not easily recognized by either nucleases or proteases, making them resistant to degradation by enzymes. PNAs are also stable over a wide pH range. In some aspects, the PNAs described herein have improved cytosolic delivery over other PNAs.

### 4. 5′ (E)-Vinyl-Phosphonate (VP) Modification

In some aspects, the nucleic acid of the disclosure, such as the RNA targeting molecule comprises one or more 5′ (E)-vinyl-phosphonate (VP) modifications. 5′-Vinyl-phosphonate modifications (metabolically stable phosphate mimics) have been reported to enhance the metabolic stability and the potency of oligonucleotides.

### 5. Morpholinos

In some aspects, the nucleic acid of the disclosure, such as the RNA targeting molecule comprises a morpholino. Morpholinos are synthetic molecules that are the product of a redesign of natural nucleic acid structure. Usually 25 bases in length, they bind to complementary sequences of RNA or single-stranded DNA by standard nucleic acid base-pairing. In terms of structure, the difference between Morpholinos and DNA is that, while Morpholinos have standard nucleic acid bases, those bases are bound to methylenemorpholine rings linked through phosphorodiamidate groups instead of phosphates. The figure compares the structures of the two strands depicted there, one of RNA and the other of a Morpholino. Replacement of anionic phosphates with the uncharged phosphorodiamidate groups eliminates ionization in the usual physiological pH range, so Morpholinos in organisms or cells are uncharged molecules. The entire backbone of a Morpholino is made from these modified subunits.

### V. Delivery Vehicles

The current disclosure contemplates several delivery systems compatible with nucleic acids that provide for roughly uniform distribution and have controllable rates of release. A variety of different media are described below that are useful in creating nucleic acid delivery systems. It is not intended that any one medium or carrier is limiting to the present invention. Note that any medium or carrier may be combined with another medium or carrier; for example, in one aspect a polymer microparticle carrier attached to a compound may be combined with a gel medium.

Carriers or mediums contemplated by this disclosure comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly (ortho esters), cyanoacrylates, gelatin-resorcinol-aldehyde type bioadhesives, polyacrylic acid and copolymers and block copolymers thereof.

a. Microparticles

Some aspects of the present disclosure contemplate a delivery system comprising a microparticle. Preferably, microparticles comprise liposomes, nanoparticles, microspheres, nanospheres, microcapsules, and nanocapsules. Preferably, some microparticles contemplated by the present invention comprise poly (lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, pseudo-poly (amino acids), polyhydroxybutyrate-related copolymers, polyanhydrides, polymethylmethacrylate, poly (ethylene oxide), lecithin and phospholipids.

### B. Liposomes

One aspect of the disclosure contemplates liposomes capable of attaching and releasing nucleic acids conjugates, polypeptides, and fusion proteins as described herein. Liposomes are microscopic spherical lipid bilayers surrounding an aqueous core that are made from amphiphilic molecules such as phospholipids. For example, a liposome may trap a nucleic acid between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phospholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One aspect of the present invention contemplates an ultra high-shear technology to refine liposome production, resulting in stable, unilamellar (single layer) liposomes having specifically designed structural characteristics. These unique properties of liposomes allow the simultaneous storage of normally immiscible compounds and the capability of their controlled release.

In some aspects, the disclosure contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

One aspect of the present disclosure contemplates a delivery system comprising liposomes that provides controlled release of at least one molecule described herein. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

### C. Microspheres, Microparticles and Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease™, Alkerme's: Cambridge, Mass.). For example, a freeze dried medium comprising at least one therapeutic agent is homogenized in a suitable solvent and sprayed to manufacture microspheres in the range of 20 to 90 μm Techniques are then followed that maintain sustained release integrity during phases of purification, encapsulation and storage. Scott et al., Improving Protein Therapeutics With Sustained Release Formulations, Nature Biotechnology, Volume 16:153-157 (1998). Modification of the microsphere composition by the use of biodegradable polymers can provide an ability to control the rate of nucleic acid release. Miller et al., Degradation Rates of Oral Resorbable Implants {Polylactates and Polyglycolates: Rate Modification and Changes in PLA/PGA Copolymer Ratios, J. Biomed. Mater. Res., Vol. 11:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a nucleic acid is added to the biodegradable polymer metal salt solution. The weight ratio of a nucleic acid to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and nucleic acid is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and nucleic acid mixture are: i) phase separation during a gradual addition of a coacervating agent; ii) an in-water drying method or phase separation method, where an antiflocculant is added to prevent particle agglomeration and iii) by a spray-drying method.

In one aspect, the present invention contemplates a medium comprising a microsphere or microcapsule capable of delivering a controlled release of a nucleic acid for a duration of approximately between 1 day and 6 months. In one aspect, the microsphere or microparticle may be colored to allow the medical practitioner the ability to see the medium clearly as it is dispensed. In another aspect, the microsphere or microcapsule may be clear. In another aspect, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere™ (Macromed) is a controlled release microsphere system. These particular microsphere's are available in uniform sizes ranging between 5-500 μm and composed of biocompatible and biodegradable polymers. Specific polymer compositions of a microsphere can control the nucleic acid release rate such that custom-designed microspheres are possible, including effective management of the burst effect. ProMaxx™ (Epic Therapeutics, Inc.) is a protein-matrix delivery system. The system is aqueous in nature and is adaptable to standard pharmaceutical delivery models. In particular, ProMaxx™ are bioerodible protein microspheres that deliver both small and macromolecular drugs, and may be customized regarding both microsphere size and desired release characteristics.

In one aspect, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated nucleic acid, therefore, will be selected for the pH environment in which dissolution is desired and stored in a pH preselected to maintain stability. Examples of pH sensitive material useful as encapsulants are Eudragit™ L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one aspect, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one aspect, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a nucleic acid is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

In one aspect, the present invention contemplates microparticles formed by spray-drying a composition comprising fibrinogen or thrombin with a nucleic acid. Preferably, these microparticles are soluble and the selected protein (i.e., fibrinogen or thrombin) creates the walls of the microparticles. Consequently, the nucleic acids are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area.

One having skill in the art will understand that the shape of the microspheres need not be exactly spherical; only as very small particles capable of being sprayed or spread into or onto a surgical site (i.e., either open or closed). In one aspect, microparticles are comprised of a biocompatible and/or biodegradable material selected from the group consisting of polylactide, polyglycolide and copolymers of lactide/glycolide (PLGA), hyaluronic acid, modified polysaccharides and any other well known material.

### VI. Proteinaceous and Nucleic Acid Compositions

The polypeptides or polynucleotides of the disclosure, such as the CIRT fusion proteins, stabilizer polypeptide, linker, RNA hairpin binding domain, NES, effector domain, tag, NLS, RNA targeting molecule, hairpin region of the RNA targeting molecule, helical region, or targeting region of the RNA targeting molecule may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOs: 1-169.

The polypeptides or polynucleotides of the disclosure, such as the CIRT fusion proteins, stabilizer polypeptide, linker, RNA hairpin binding domain, NES, effector domain, tag, NLS, RNA targeting molecule, hairpin region of the RNA targeting molecule, helical region, or targeting region of the RNA targeting molecule may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of SEQ ID NOs: 1-169.

In some aspects, the polypeptide or nucleic acid may comprise amino acids or nucleotide 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) of SEQ ID NOs: 1-169.

In some aspects, the polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids or nucleic acids of SEQ ID NOs: 1-169.

In some aspects, the polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOs: 1-169 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NOs: 1-169.

In other aspects, the stabilizer polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90, or any derivable range therein of any one of SEQ ID NOs: 14, 58, or 59.

In some aspects, the stabilizer polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 (or any derivable range therein) contiguous amino acids of any one of SEQ ID NOs: 14, 58, or 59.

In some aspects, the stabilizer polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 (or any derivable range therein) contiguous amino acids of any one of SEQ ID NOs: 14, 58, or 59 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of any one of SEQ ID NOs: 14, 58, or 59.

In some aspects, the RNA hairpin binding domain may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, or 102, (or any derivable range therein) of SEQ ID NO:16.

In some aspects, the RNA hairpin binding domain may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, or 102 (or any derivable range therein) contiguous amino acids of SEQ ID NO:16.

In some aspects, the RNA hairpin binding domain may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, or 102 (or any derivable range therein) contiguous amino acids of SEQ ID NO:16. that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NO:16.

In some aspects, the effector domain may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, or 364 (or any derivable range therein) of SEQ ID NOs: 19-39, 60-62, 69-76, or 136-143.

In some aspects, the effector domain may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, or 364 (or any derivable range therein) contiguous amino acids SEQ ID NOs: 19-39, 60-62, 69-76, 113-121, or 136-143.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 of any of SEQ ID NOs: 1-169 and comprising 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 contiguous nucleotides or amino acids of any of SEQ ID NOs: 1-169.

In some aspects, the effector domain may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, or 364 (or any derivable range therein) contiguous amino acids of SEQ ID NOs: 19-39, 60-62, 69-76, 113-121, or 136-143 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NOs: 19-39, 60-62, 69-76, 113-121, or 136-143.

In some aspects, the hairpin structure, such as the stem, loop, or both stem and loop, of the RNA targeting molecule may comprise nucleic acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43 (or any derivable range therein) of SEQ ID NOs: 40-57.

In some aspects, the hairpin structure, such as the stem, loop, or both stem and loop, of the RNA targeting molecule may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43 (or any derivable range therein) contiguous nucleic acids of SEQ ID NOs: 40-57.

In some aspects, the hairpin structure, such as the stem, loop, or both stem and loop, of the RNA targeting molecule may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43 (or any derivable range therein) contiguous nucleic acids of SEQ ID NOs: 40-57 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar or identical with one of SEQ ID NOs: 40-57.

In some aspects, the stabilizer polypeptide may have one or more substitutions that reduce or eliminate binding to endogenous proteins. In some aspects, the stabilizer polypeptide may have one or more substitutions that reduce or eliminate an activity directed to an endogenous protein.

The polypeptides and nucleic acids of the disclosure, such as the CIRT proteins, stabilizer domain, linker, RNA hairpin binding domain, NES, effector domain, molecular tag, RNA hairpin region of the RNA targeting molecule, may comprise, comprise at least, or comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions.

The substitution may be at amino acid position or nucleic acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 of one of SEQ ID NO:1-169.

The substitution at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 of SEQ ID NOs: 1-169 may be a substitution with an alanine, arginine, asparagine, aspartic acid, cysteine, glutamine, glutamic acid, glycine, histidine, isoleucine, leusine, lysine, methionine, phenylalanine, proline, serine, threonine, tryptophan, tyrosine, or valine.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other aspects, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific aspects, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One aspect includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### VII. Sequences

| | | SEQ |
|---|---|---|
| RNA targeting molecule- | | ID |
| hairpin domain | Sequence | NO: |
| TAR hairpin Scaffold | ggccagaucugagccugggagcucucuggcc | 40 |
| Human SLBP hairpin | ccaaaggcucuucucagagccaccca | 41 |
| scaffold | | |

| Description | Sequence | Seq ID |
|---|---|---|
| B-defensin | MGIINTLQKYYCRVRGGRCAVLSCLPKEEQIGKCSTRGRKCCR | 14 |
| | RKK | |
| Linker | GGSGGSGGSGGSGGSGGS | 15 |
| TBP6.7 | MAVPETRPNHTIYINNLNSKIKKDELKKSLYAIFSQFGQILDILVP | 16 |
| | RQRTPRGQAFVIFKEVSSATNALRSMQGFPFYDKPMRIQYAKT | |
| | DKRIPAKMKGTFV | |
| linker | GS | |
| HIV NES | LQLPPLERLTL | 17 |
| FLAG | DYKDDDDK | 18 |
| CIRT-1 | MGIINTLQKYYCRVRGGRCAVLSCLPKEEQIGKCSTRGRKCCR | 1 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMDLTAIYESLLSLSPDVPVPS | |
| | DHGGTESSPGWGSSGPWSLSPSDSSPSGVTSRLPGRSTSLVEGR | |
| | SCGWVPPPPGFAPLAPRLGPELSPSPTSPTATSTTPSRYKTELCR | |
| | TFSESGRCRYGAKCQFAHGLGELRQANRHPKYKTELCHKFYLQ | |
| | GRCPYGSRCHFIHNPSEDLAAPGHPPVLRQSISFSGLPSGRRTSPP | |
| | PPGLAGPSLSSSSFSPSSSPPPPGDLPLSPSAFSAAPGTPLARRDPT | |
| | PVCCPSCRRATPISVWGPLGGLVRTPSVQSLGSDPDEYASSGSS | |
| | LGGSDSPVFEAGVFAPPQPVAAPRRLPIFNRISVSEGGSGGSGGS | |
| | GGSGGSGGSDAMFGQPGALGSTPFLGQHGFNFFPSGIDFSAWG | |
| | NNSSQGQSTQSSGYSSNYAYAPSSLGGAMIDGQSAFANETLNK | |
| | APGMNTIDQGMAAGSDYKDDDDK* | |
| ZFP36 | MDLTAIYESLLSLSPDVPVPSDHGGTESSPGWGSSGPWSLSPSD | 32 |
| | SSPSGVTSRLPGRSTSLVEGRSCGWVPPPPGFAPLAPRLGPELSP | |
| | SPTSPTATSTTPSRYKTELCRTFSESGRCRYGAKCQFAHGLGEL | |
| | RQANRHPKYKTELCHKFYLQGRCPYGSRCHFIHNPSEDLAAPG | |
| | HPPVLRQSISFSGLPSGRRTSPPPPGLAGPSLSSSSFSPSSSPPPPG | |
| | DLPLSPSAFSAAPGTPLARRDPTPVCCPSCRRATPISVWGPLGGL | |
| | VRTPSVQSLGSDPDEYASSGSSLGGSDSPVFEAGVFAPPQPVAA | |
| | PRRLPIFNRISVSE | |
| YTHDF2 | DAMFGQPGALGSTPFLGQHGFNFFPSGIDFSAWGNNSSQGQST | 33 |
| | QSSGYSSNYAYAPSSLGGAMIDGQSAFANETLNKAPGMNTIDQ | |
| | GMAA | |
| ZFP36- | MDLTAIYESLLSLSPDVPVPSDHGGTESSPGWGSSGPWSLSPSD | 19 |
| YTHDF2 | SSPSGVTSRLPGRSTSLVEGRSCGWVPPPPGFAPLAPRLGPELSP | |
| | SPTSPTATSTTPSRYKTELCRTFSESGRCRYGAKCQFAHGLGEL | |
| | RQANRHPKYKTELCHKFYLQGRCPYGSRCHFIHNPSEDLAAPG | |
| | HPPVLRQSISFSGLPSGRRTSPPPPGLAGPSLSSSSFSPSSSPPPPG | |
| | DLPLSPSAFSAAPGTPLARRDPTPVCCPSCRRATPISVWGPLGGL | |
| | VRTPSVQSLGSDPDEYASSGSSLGGSDSPVFEAGVFAPPQPVAA | |
| | PRRLPIFNRISVSEGGSGGSGGSGGSGGSGGSDAMFGQPGALGS | |
| | TPFLGQHGFNFFPSGIDFSAWGNNSSQGQSTQSSGYSSNYAYAP | |
| | SSLGGAMIDGQSAFANETLNKAPGMNTIDQGMAA | |
| CIRT-2 | MGIINTLQKYYCRVRGGRCAVLSCLPKEEQIGKCSTRGRKCCR | 2 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMPAATVDHSQRICEVWACNL | |
| | DEEMKKIRQVIRKYNYVAMDTEFPGVVARPIGEFRSNADYQYQ | |
| | LLRCNVDLLKIIQLGLTFMNEQGEYPPGTSTWQFNFKFNLTEDM | |
| | YAQDSIELLTTSGIQFKKHEEEGIETQYFAELLMTSGVVLCEGV | |
| | KWLSFHSGYDFGYLIKILTNSNLPEEELDFFEILRLFFPVIYDVKY | |
| | LMKSCKNLKGGLQEVAEQLELERIGPQHQAGSDSLLTGMAFFK | |
| | MREMFFEDHIDDAKYCGHLYGLGSGSSYVQNGTGNAYEEEAN | |
| | KQSGGSGGSGGSGGSGGSGGSDAMFGQPGALGSTPFLGQHGF | |
| | NFFPSGIDFSAWGNNSSQGQSTQSSGYSSNYAYAPSSLGGAMID | |
| | GQSAFANETLNKAPGMNTIDQGMAAGSDYKDDDDK* | |
| CNOT7 | MPAATVDHSQRICEVWACNLDEEMKKIRQVIRKYNYVAMDTE | 34 |
| | FPGVVARPIGEFRSNADYQYQLLRCNVDLLKIIQLGLTFMNEQG | |
| | EYPPGTSTWQFNFKFNLTEDMYAQDSIELLTTSGIQFKKHEEEGI | |
| | ETQYFAELLMTSGVVLCEGVKWLSFHSGYDFGYLIKILTNSNLP | |
| | EEELDFFEILRLFFPVIYDVKYLMKSCKNLKGGLQEVAEQLELE | |
| | RIGPQHQAGSDSLLTGMAFFKMREMFFEDHIDDAKYCGHLYGL | |
| | GSGSSYVQNGTGNAYEEEANKQS | |
| YTHDF2 | DAMFGQPGALGSTPFLGQHGFNFFPSGIDFSAWGNNSSQGQST | 33 |
| | QSSGYSSNYAYAPSSLGGAMIDGQSAFANETLNKAPGMNTIDQ | |
| | GMAA | |
| CNOT7- | MPAATVDHSQRICEVWACNLDEEMKKIRQVIRKYNYVAMDTE | 20 |
| YTHDF2 | FPGVVARPIGEFRSNADYQYQLLRCNVDLLKIIQLGLTFMNEQG | |
| | EYPPGTSTWQFNFKFNLTEDMYAQDSIELLTTSGIQFKKHEEEGI | |
| | ETQYFAELLMTSGVVLCEGVKWLSFHSGYDFGYLIKILTNSNLP | |
| | EEELDFFEILRLFFPVIYDVKYLMKSCKNLKGGLQEVAEQLELE | |
| | RIGPQHQAGSDSLLTGMAFFKMREMFFEDHIDDAKYCGHLYGL | |
| | GSGSSYVQNGTGNAYEEEANKQSGGSGGSGGSGGSGGSGGSD | |
| | AMFGQPGALGSTPFLGQHGFNFFPSGIDFSAWGNNSSQGQSTQS | |
| | SGYSSNYAYAPSSLGGAMIDGQSAFANETLNKAPGMNTIDQG | |
| | MAA | |
| CIRT-3 | MGIINTLQKYYCRVRGGRCAVLSCLPKEEQIGKCSTRGRKCCR | 3 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMPAATVDHSQRICEVWACNL | |
| | DEEMKKIRQVIRKYNYVAMDTEFPGVVARPIGEFRSNADYQYQ | |
| | LLRCNVDLLKIIQLGLTFMNEQGEYPPGTSTWQFNFKFNLTEDM | |
| | YAQDSIELLTTSGIQFKKHEEEGIETQYFAELLMTSGVVLCEGV | |
| | KWLSFHSGYDFGYLIKILTNSNLPEEELDFFEILRLFFPVIYDVKY | |
| | LMKSCKNLKGGLQEVAEQLELERIGPQHQAGSDSLLTGMAFFK | |
| | MREMFFEDHIDDAKYCGHLYGLGSGSSYVQNGTGNAYEEEAN | |
| | KQSGGSGGSGGSGGSGGSGGSMDLTAIYESLLSLSPDVPVPSDH | |
| | GGTESSPGWGSSGPWSLSPSDSSPSGVTSRLPGRSTSLVEGRSCG | |
| | WVPPPPGFAPLAPRLGPELSPSPTSPTATSTTPSRYKTELCRTFSE | |
| | SGRCRYGAKCQFAHGLGELRQANRHPKYKTELCHKFYLQGRC | |
| | PYGSRCHFIHNPSEDLAAPGHPPVLRQSISFSGLPSGRRTSPPPPG | |
| | LAGPSLSSSSFSPSSSPPPPGDLPLSPSAFSAAPGTPLARRDPTPVC | |
| | CPSCRRATPISVWGPLGGLVRTPSVQSLGSDPDEYASSGSSLGG | |
| | SDSPVFEAGVFAPPQPVAAPRRLPIFNRISVSEGSDYKDDDDK* | |
| CNOT7 | MPAATVDHSQRICEVWACNLDEEMKKIRQVIRKYNYVAMDTE | 34 |
| | FPGVVARPIGEFRSNADYQYQLLRCNVDLLKIIQLGLTFMNEQG | |
| | EYPPGTSTWQFNFKFNLTEDMYAQDSIELLTTSGIQFKKHEEEGI | |
| | ETQYFAELLMTSGVVLCEGVKWLSFHSGYDFGYLIKILTNSNLP | |
| | EEELDFFEILRLFFPVIYDVKYLMKSCKNLKGGLQEVAEQLELE | |
| | RIGPQHQAGSDSLLTGMAFFKMREMFFEDHIDDAKYCGHLYGL | |
| | GSGSSYVQNGTGNAYEEEANKQS | |
| ZFP36 | MDLTAIYESLLSLSPDVPVPSDHGGTESSPGWGSSGPWSLSPSD | 32 |
| | SSPSGVTSRLPGRSTSLVEGRSCGWVPPPPGFAPLAPRLGPELSP | |
| | SPTSPTATSTTPSRYKTELCRTFSESGRCRYGAKCQFAHGLGEL | |
| | RQANRHPKYKTELCHKFYLQGRCPYGSRCHFIHNPSEDLAAPG | |
| | HPPVLRQSISFSGLPSGRRTSPPPPGLAGPSLSSSSFSPSSSPPPPG | |
| | DLPLSPSAFSAAPGTPLARRDPTPVCCPSCRRATPISVWGPLGGL | |
| | VRTPSVQSLGSDPDEYASSGSSLGGSDSPVFEAGVFAPPQPVAA | |
| | PRRLPIFNRISVSE | |
| CNOT7- | MPAATVDHSQRICEVWACNLDEEMKKIRQVIRKYNYVAMDTE | 21 |
| ZFP36 | FPGVVARPIGEFRSNADYQYQLLRCNVDLLKIIQLGLTFMNEQG | |
| | EYPPGTSTWQFNFKFNLTEDMYAQDSIELLTTSGIQFKKHEEEGI | |
| | ETQYFAELLMTSGVVLCEGVKWLSFHSGYDFGYLIKILTNSNLP | |
| | EEELDFFEILRLFFPVIYDVKYLMKSCKNLKGGLQEVAEQLELE | |
| | RIGPQHQAGSDSLLTGMAFFKMREMFFEDHIDDAKYCGHLYGL | |
| | GSGSSYVQNGTGNAYEEEANKQSGGSGGSGGSGGSGGSGGSM | |
| | DLTAIYESLLSLSPDVPVPSDHGGTESSPGWGSSGPWSLSPSDSS | |
| | PSGVTSRLPGRSTSLVEGRSCGWVPPPPGFAPLAPRLGPELSPSP | |
| | TSPTATSTTPSRYKTELCRTFSESGRCRYGAKCQFAHGLGELRQ | |
| | ANRHPKYKTELCHKFYLQGRCPYGSRCHFIHNPSEDLAAPGHP | |
| | PVLRQSISFSGLPSGRRTSPPPPGLAGPSLSSSSFSPSSSPPPPGDLP | |
| | LSPSAFSAAPGTPLARRDPTPVCCPSCRRATPISVWGPLGGLVRT | |
| | PSVQSLGSDPDEYASSGSSLGGSDSPVFEAGVFAPPQPVAAPRR | |
| | LPIFNRISVSE | |
| CIRT-4 | MGIINTLQKYYCRVRGGRCAVLSCLPKEEQIGKCSTRGRKCCR | 4 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSTRSRYASDFEEIAVLGQGAFG | |
| | QVVKARNALDSRYYAIKKIRHTEEKLSTILSEVMLLASLNHQYV | |
| | VRYYAAWLEEDSMDENVFESTDEESDLSESSSDFEENDLLDQSS | |
| | IFKNRTNHDLDNSNWDFISGSGYPDIVFENSSRDDENEDLDHDT | |
| | SSTSSSESQDDTDKESKSIQNVPRRRNFVKPMTAVKKKSTLFIQ | |
| | MEYCENGTLYDLIHSENLNQQRDEYWRLFRQILEALSYIHSQGII | |
| | HRDLKPMNILIDESRNVKIGDFGLAKNVHRSLDILKLDSQNLPG | |
| | SSDNLTSAIGTAMYVATEVLDGTGHYNEKIDMYSLGIIFFEMIY | |
| | PFSTGMERVNILKKLRSVSIEFPPDFDDNKMKVEKKIIRLLIDHD | |
| | PNKRPGARTLLNSGWLPVKHQDEVIKEALKSLSNPSSPWQQQV | |
| | REGSDYKDDDDK* | |
| GCN2 | TRSRYASDFEEIAVLGQGAFGQVVKARNALDSRYYAIKKIRHTE | 22 |
| | EKLSTILSEVMLLASLNHQYVVRYYAAWLEEDSMDENVFESTD | |
| | EESDLSESSSDFEENDLLDQSSIFKNRTNHDLDNSNWDFISGSGY | |
| | PDIVFENSSRDDENEDLDHDTSSTSSSESQDDTDKESKSIQNVPR | |
| | RRNFVKPMTAVKKKSTLFIQMEYCENGTLYDLIHSENLNQQRD | |
| | EYWRLFRQILEALSYIHSQGIIHRDLKPMNILIDESRNVKIGDFGL | |
| | AKNVHRSLDILKLDSQNLPGSSDNLTSAIGTAMYVATEVLDGT | |
| | GHYNEKIDMYSLGIIFFEMIYPFSTGMERVNILKKLRSVSIEFPPD | |
| | FDDNKMKVEKKIIRLLIDHDPNKRPGARTLLNSGWLPVKHQDE | |
| | VIKEALKSLSNPSSPWQQQVRE | |
| CIRT-5 | MGIINTLQKYYCRVRGGRCAVLSCLPKEEQIGKCSTRGRKCCR | 5 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMSGGSSCSQTPSRAIPTRRVA | |
| | LGDGVQLPPGDYSTAPGGTLFSTAPGGTRIIYDRKFLMECRNAP | |
| | VAKAPPKDLPTIPGVTSPTSDEPPMQASQSHLHSSPEDKRAGGE | |
| | ESQFEMDIGSDYKDDDDK* | |
| 4EBP1 | MSGGSSCSQTPSRAIPTRRVALGDGVQLPPGDYSTAPGGTLFST | 23 |
| | APGGTRIIYDRKFLMECRNAPVAKAPPKDLPTIPGVTSPTSDEPP | |
| | MQASQSHLHSSPEDKRAGGEESQFEMDI | |
| CIRT-6 | MGIINTLQKYYCRVRGGRCAVLSCLPKEEQIGKCSTRGRKCCR | 6 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMGTFDLWTDYLGLAHLVRA | |
| | LSGKEGPETRLSPQPEPEPMLEPVSALEPMPAPESVPVPGPKDQ | |
| | KRSLESSPAPERLCSFCKHNGESRAIYQSHVLKDEAGRVLCPILR | |
| | DYVCPQCGATRERAHTRRFCPLTGQGYTSVYSHTTRNSAGKKL | |
| | VRPDKAKTQDTGHRRGGGGGAGFRGAGKSEPSPSCSPSMSTGS | |
| | DYKDDDDK* | |
| NANOS3 | MGTFDLWTDYLGLAHLVRALSGKEGPETRLSPQPEPEPMLEPV | 24 |
| | SALEPMPAPESVPVPGPKDQKRSLESSPAPERLCSFCKHNGESR | |
| | AIYQSHVLKDEAGRVLCPILRDYVCPQCGATRERAHTRRFCPLT | |
| | GQGYTSVYSHTTRNSAGKKLVRPDKAKTQDTGHRRGGGGGA | |
| | GFRGAGKSEPSPSCSPSMST | |
| CIRT-7 | MGIINTLQKYYCRVRGGRCAVLSCLPKEEQIGKCSTRGRKCCR | 7 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMATVEPETTPTPNPPTTEEEK | |
| | TESNQEVANPEHYIKHPLQNRWALWFFKNDKSKTWQANLRLIS | |
| | KFDTVEDFWALYNHIQLSSNLMPGCDYSLFKDGIEPMWEDEKN | |
| | KRGGRWLITLNKQQRRSDLDRFWLETLLCLIGESFDDYSDDVC | |
| | GAVVNVRAKGDKIAIWTTECENREAVTHIGRVYKERLGLPPKI | |
| | VIGYQSHADTATKSGSTTKNRFVVGGSGGSGGSGGSGGSGGSM | |
| | SASQDSRSRDNGPDGMEPEGVIESNWNEIVDSFDDMNLSESLLR | |
| | GIYAYGFEKPSAIQQRAILPCIKGYDVIAQAQSGTGKTATFAISIL | |
| | QQIELDLKATQALVLAPTRELAQQIQKVVMALGDYMGASCHA | |
| | CIGGTNVRAEVQKLQMEAPHIIVGTPGRVFDMLNRRYLSPKYIK | |
| | MFVLDEADEMLSRGFKDQIYDIFQKLNSNTQVVLLSATMPSDV | |
| | LEVTKKFMRDPIRILVKKEELTLEGIRQFYINVEREEWKLDTLC | |
| | DLYETLTITQAVIFINTRRKVDWLTEKMHARDFTVSAMHGDMD | |
| | QKERDVIMREFRSGSSRVLITTDLLARGIDVQQVSLVINYDLPTN | |
| | RENYIHRIGRGGRFGRKGVAINMVTEEDKRTLRDIETFYNTSIEE | |
| | MPLNVADLIGSDYKDDDDK* | |
| EIF4E | MATVEPETTPTPNPPTTEEEKTESNQEVANPEHYIKHPLQNRWA | 35 |
| | LWFFKNDKSKTWQANLRLISKFDTVEDFWALYNHIQLSSNLMP | |
| | GCDYSLFKDGIEPMWEDEKNKRGGRWLITLNKQQRRSDLDRF | |
| | WLETLLCLIGESFDDYSDDVCGAVVNVRAKGDKIAIWTTECEN | |
| | REAVTHIGRVYKERLGLPPKIVIGYQSHADTATKSGSTTKNRFV | |
| | V | |
| EIF4A | MSASQDSRSRDNGPDGMEPEGVIESNWNEIVDSFDDMNLSESL | 36 |
| | LRGIYAYGFEKPSAIQQRAILPCIKGYDVIAQAQSGTGKTATFAI | |
| | SILQQIELDLKATQALVLAPTRELAQQIQKVVMALGDYMGASC | |
| | HACIGGTNVRAEVQKLQMEAPHIIVGTPGRVFDMLNRRYLSPK | |
| | YIKMFVLDEADEMLSRGFKDQIYDIFQKLNSNTQVVLLSATMPS | |
| | DVLEVTKKFMRDPIRILVKKEELTLEGIRQFYINVEREEWKLDT | |
| | LCDLYETLTITQAVIFINTRRKVDWLTEKMHARDFTVSAMHGD | |
| | MDQKERDVIMREFRSGSSRVLITTDLLARGIDVQQVSLVINYDL | |
| | PTNRENYIHRIGRGGRFGRKGVAINMVTEEDKRTLRDIETFYNT | |
| | SIEEMPLNVADLI | |
| EIF4E- | MATVEPETTPTPNPPTTEEEKTESNQEVANPEHYIKHPLQNRWA | 25 |
| EIF4A | LWFFKNDKSKTWQANLRLISKFDTVEDFWALYNHIQLSSNLMP | |
| | GCDYSLFKDGIEPMWEDEKNKRGGRWLITLNKQQRRSDLDRF | |
| | WLETLLCLIGESFDDYSDDVCGAVVNVRAKGDKIAIWTTECEN | |
| | REAVTHIGRVYKERLGLPPKIVIGYQSHADTATKSGSTTKNRFV | |
| | VGGSGGSGGSGGSGGSGGSMSASQDSRSRDNGPDGMEPEGVIE | |
| | SNWNEIVDSFDDMNLSESLLRGIYAYGFEKPSAIQQRAILPCIKG | |
| | YDVIAQAQSGTGKTATFAISILQQIELDLKATQALVLAPTRELA | |
| | QQIQKVVMALGDYMGASCHACIGGTNVRAEVQKLQMEAPHII | |
| | VGTPGRVFDMLNRRYLSPKYIKMFVLDEADEMLSRGFKDQIYD | |
| | IFQKLNSNTQVVLLSATMPSDVLEVTKKFMRDPIRILVKKEELT | |
| | LEGIRQFYINVEREEWKLDTLCDLYETLTITQAVIFINTRRKVDW | |
| | LTEKMHARDFTVSAMHGDMDQKERDVIMREFRSGSSRVLITTD | |
| | LLARGIDVQQVSLVINYDLPTNRENYIHRIGRGGRFGRKGVAIN | |
| | MVTEEDKRTLRDIETFYNTSIEEMPLNVADLI | |
| CIRT-8 | MGIINTLQKYYCRVRGGRCAVLSCLPKEEQIGKCSTRGRKCCR | 8 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMSASQDSRSRDNGPDGMEPE | |
| | GVIESNWNEIVDSFDDMNLSESLLRGIYAYGFEKPSAIQQRAILP | |
| | CIKGYDVIAQAQSGTGKTATFAISILQQIELDLKATQALVLAPTR | |
| | ELAQQIQKVVMALGDYMGASCHACIGGTNVRAEVQKLQMEA | |
| | PHIIVGTPGRVFDMLNRRYLSPKYIKMFVLDEADEMLSRGFKD | |
| | QIYDIFQKLNSNTQVVLLSATMPSDVLEVTKKFMRDPIRILVKK | |
| | EELTLEGIRQFYINVEREEWKLDTLCDLYETLTITQAVIFINTRR | |
| | KVDWLTEKMHARDFTVSAMHGDMDQKERDVIMREFRSGSSR | |
| | VLITTDLLARGIDVQQVSLVINYDLPTNRENYIHRIGRGGRFGRK | |
| | GVAINMVTEEDKRTLRDIETFYNTSIEEMPLNVADLIGGSGGSG | |
| | GSGGSGGSGGSMSDGFDRAPGAGRGRSRGLGRGGGGPEGGGF | |
| | PNGAGPAERARHQPPQPKAPGFLQPPPLRQPRTTPPPGAQCEVP | |
| | ASPQRPSRPGALPEQTRPLRAPPSSQDKIPQQNSESAMAKPQVV | |
| | VAPVLMSKLSVNAPEFYPSGYSSSYTESYEDGCEDYPTLSEYVQ | |
| | DFLNHLTEQPGSFETEIEQFAETLNGCVTTDDALQELVELIYQQ | |
| | ATSIPNFSYMGARLCNYLSHHLTISPQSGNFRQLLLQRCRTEYE | |
| | VKDQAAKGDEVTRKRFHAFVLFLGELYLNLEIKGTNGQVTRA | |
| | DILQVGLRELLNALFSNPMDDNLICAVKLLKLTGSVLEDAWKE | |
| | KGKMDMEEIIQRIENVVLDANCSRDVKQMLLKLVELRSSNWG | |
| | RVHATSTYREATPENDPNYFMNEPTFYTSDGVPFTAADPDYQE | |
| | KYQELLEREDFFPDYEENGTDLSGAGDPYLDDIDDEMDPEIEEA | |
| | YEKFCLESERKRKQGSDYKDDDDK* | |
| EIF4A | MSASQDSRSRDNGPDGMEPEGVIESNWNEIVDSFDDMNLSESL | 36 |
| | LRGIYAYGFEKPSAIQQRAILPCIKGYDVIAQAQSGTGKTATFAI | |
| | SILQQIELDLKATQALVLAPTRELAQQIQKVVMALGDYMGASC | |
| | HACIGGTNVRAEVQKLQMEAPHIIVGTPGRVEDMLNRRYLSPK | |
| | YIKMFVLDEADEMLSRGFKDQIYDIFQKLNSNTQVVLLSATMPS | |
| | DVLEVTKKFMRDPIRILVKKEELTLEGIRQFYINVEREEWKLDT | |
| | LCDLYETLTITQAVIFINTRRKVDWLTEKMHARDFTVSAMHGD | |
| | MDQKERDVIMREFRSGSSRVLITTDLLARGIDVQQVSLVINYDL | |
| | PTNRENYIHRIGRGGRFGRKGVAINMVTEEDKRTLRDIETFYNT | |
| | SIEEMPLNVADLI | |
| PAIP | MSDGFDRAPGAGRGRSRGLGRGGGGPEGGGFPNGAGPAERAR | 37 |
| | HQPPQPKAPGFLQPPPLRQPRTTPPPGAQCEVPASPQRPSRPGAL | |
| | PEQTRPLRAPPSSQDKIPQQNSESAMAKPQVVVAPVLMSKLSV | |
| | NAPEFYPSGYSSSYTESYEDGCEDYPTLSEYVQDFLNHLTEQPG | |
| | SFETEIEQFAETLNGCVTTDDALQELVELIYQQATSIPNFSYMGA | |
| | RLCNYLSHHLTISPQSGNFRQLLLQRCRTEYEVKDQAAKGDEV | |
| | TRKRFHAFVLFLGELYLNLEIKGTNGQVTRADILQVGLRELLNA | |
| | LFSNPMDDNLICAVKLLKLTGSVLEDAWKEKGKMDMEEIIQRI | |
| | ENVVLDANCSRDVKQMLLKLVELRSSNWGRVHATSTYREATP | |
| | ENDPNYFMNEPTFYTSDGVPFTAADPDYQEKYQELLEREDFFP | |
| | DYEENGTDLSGAGDPYLDDIDDEMDPEIEEAYEKFCLESERKRK | |
| | Q | |
| EIF4A- | MSASQDSRSRDNGPDGMEPEGVIESNWNEIVDSFDDMNLSESL | 26 |
| PAIP | LRGIYAYGFEKPSAIQQRAILPCIKGYDVIAQAQSGTGKTATFAI | |
| | SILQQIELDLKATQALVLAPTRELAQQIQKVVMALGDYMGASC | |
| | HACIGGTNVRAEVQKLQMEAPHIIVGTPGRVFDMLNRRYLSPK | |
| | YIKMFVLDEADEMLSRGFKDQIYDIFQKLNSNTQVVLLSATMPS | |
| | DVLEVTKKFMRDPIRILVKKEELTLEGIRQFYINVEREEWKLDT | |
| | LCDLYETLTITQAVIFINTRRKVDWLTEKMHARDFTVSAMHGD | |
| | MDQKERDVIMREFRSGSSRVLITTDLLARGIDVQQVSLVINYDL | |
| | PTNRENYIHRIGRGGRFGRKGVAINMVTEEDKRTLRDIETFYNT | |
| | SIEEMPLNVADLIGGSGGSGGSGGSGGSGGSMSDGFDRAPGAG | |
| | RGRSRGLGRGGGGPEGGGFPNGAGPAERARHQPPQPKAPGFLQ | |
| | PPPLRQPRTTPPPGAQCEVPASPQRPSRPGALPEQTRPLRAPPSS | |
| | QDKIPQQNSESAMAKPQVVVAPVLMSKLSVNAPEFYPSGYSSS | |
| | YTESYEDGCEDYPTLSEYVQDFLNHLTEQPGSFETEIEQFAETLN | |
| | GCVTTDDALQELVELIYQQATSIPNFSYMGARLCNYLSHHLTIS | |
| | PQSGNFRQLLLQRCRTEYEVKDQAAKGDEVTRKRFHAFVLFLG | |
| | ELYLNLEIKGTNGQVTRADILQVGLRELLNALFSNPMDDNLICA | |
| | VKLLKLTGSVLEDAWKEKGKMDMEEIIQRIENVVLDANCSRDV | |
| | KQMLLKLVELRSSNWGRVHATSTYREATPENDPNYFMNEPTF | |
| | YTSDGVPFTAADPDYQEKYQELLEREDFFPDYEENGTDLSGAG | |
| | DPYLDDIDDEMDPEIEEAYEKFCLESERKRKQ | |
| CIRT-9 | MGIINTLQKYYCRVRGGRCAVLSCLPKEEQIGKCSTRGRKCCR | 9 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMSATSVDTQRTKGQDNKVQ | |
| | NGSLHQKDTVHDNDFEPYLTGQSNQSNSYPSMSDPYLSSYYPP | |
| | SIGFPYSLNEAPWSTAGDPPIPYLTTYGQLSNGDHHFMHDAVFG | |
| | QPGGLGNNIYQHRFNFFPENPAFSAWGTSGSQGQQTQSSAYGS | |
| | SYTYPPSSLGGTVVDGQPGFHSDTLSKAPGMNSLEQGMVGLKI | |
| | GDVSSSAVKTVGSVVSSVALTGVLSGNGGTNVNMPVSKPTSW | |
| | AAIASKPAKPQPKMKTKSGPVMGGGLPPPPIKHNMDIGTWDNK | |
| | GPVPKAPVPQQAPSPQAAPQPQQVAQPLPAQPPALAQPQYQSP | |
| | QQPPQTRWVAPRNRNAAFGQSGGAGSDSNSPGNVQPNSAPSV | |
| | ESGGSGGSGGSGGSGGSGGSMSATSVDQRPKGQGNKVSVQNG | |
| | SIHQKDAVNDDDFEPYLSSQTNQSNSYPPMSDPYMPSYYAPSIG | |
| | FPYSLGEAAWSTAGDQPMPYLTTYGQMSNGEHHYIPDGVFSQP | |
| | GALGNTPPFLGQHGFNFFPGNADFSTWGTSGSQGQSTQSSAYSS | |
| | SYGYPPSSLGRAITDGQAGFGNDTLSKVPGISSIEQGMTGLKIGG | |
| | DLTAAVTKTVGTALSSSGMTSIATNSVPPVSSAAPKPTSWAAIA | |
| | RKPAKPQPKLKPKGNVGIGGSAVPPPPIKHNMNIGTWDEKGSV | |
| | VKAPPTQPVLPPQTIIQQPQPLIQPPPLVQSQLPQQQPQPPQPQQ | |
| | QQGPQPQAQPHQVQPQQQQLQNRWVAPRNRGAGFNQNNGAG | |
| | SENFGLGVVPVSASPSSVEVHPVLEKLKAINNYNPKDFDWNLK | |
| | NGSDYKDDDDK* | |
| YTHDF1 | MSATSVDTQRTKGQDNKVQNGSLHQKDTVHDNDFEPYLTGQS | 38 |
| | NQSNSYPSMSDPYLSSYYPPSIGFPYSLNEAPWSTAGDPPIPYLT | |
| | TYGQLSNGDHHFMHDAVFGQPGGLGNNIYQHRFNFFPENPAFS | |
| | AWGTSGSQGQQTQSSAYGSSYTYPPSSLGGTVVDGQPGFHSDT | |
| | LSKAPGMNSLEQGMVGLKIGDVSSSAVKTVGSVVSSVALTGVL | |
| | SGNGGTNVNMPVSKPTSWAAIASKPAKPQPKMKTKSGPVMGG | |
| | GLPPPPIKHNMDIGTWDNKGPVPKAPVPQQAPSPQAAPQPQQV | |
| | AQPLPAQPPALAQPQYQSPQQPPQTRWVAPRNRNAAFGQSGG | |
| | AGSDSNSPGNVQPNSAPSVES | |
| YTHDF3 | MSATSVDQRPKGQGNKVSVQNGSIHQKDAVNDDDFEPYLSSQ | 39 |
| | TNQSNSYPPMSDPYMPSYYAPSIGFPYSLGEAAWSTAGDQPMP | |
| | YLTTYGQMSNGEHHYIPDGVFSQPGALGNTPPFLGQHGFNFFP | |
| | GNADFSTWGTSGSQGQSTQSSAYSSSYGYPPSSLGRAITDGQAG | |
| | FGNDTLSKVPGISSIEQGMTGLKIGGDLTAAVTKTVGTALSSSG | |
| | MTSIATNSVPPVSSAAPKPTSWAAIARKPAKPQPKLKPKGNVGI | |
| | GGSAVPPPPIKHNMNIGTWDEKGSVVKAPPTQPVLPPQTIIQQP | |
| | QPLIQPPPLVQSQLPQQQPQPPQPQQQQGPQPQAQPHQVQPQQ | |
| | QQLQNRWVAPRNRGAGFNQNNGAGSENFGLGVVPVSASPSSV | |
| | EVHPVLEKLKAINNYNPKDFDWNLKN | |
| YTHDF1- | MSATSVDTQRTKGQDNKVQNGSLHQKDTVHDNDFEPYLTGQS | 27 |
| YTHDF3 | NQSNSYPSMSDPYLSSYYPPSIGFPYSLNEAPWSTAGDPPIPYLT | |
| | TYGQLSNGDHHFMHDAVFGQPGGLGNNIYQHRFNFFPENPAFS | |
| | AWGTSGSQGQQTQSSAYGSSYTYPPSSLGGTVVDGQPGFHSDT | |
| | LSKAPGMNSLEQGMVGLKIGDVSSSAVKTVGSVVSSVALTGVL | |
| | SGNGGTNVNMPVSKPTSWAAIASKPAKPQPKMKTKSGPVMGG | |
| | GLPPPPIKHNMDIGTWDNKGPVPKAPVPQQAPSPQAAPQPQQV | |
| | AQPLPAQPPALAQPQYQSPQQPPQTRWVAPRNRNAAFGQSGG | |
| | AGSDSNSPGNVQPNSAPSVESGGSGGSGGSGGSGGSGGSMSAT | |
| | SVDQRPKGQGNKVSVQNGSIHQKDAVNDDDFEPYLSSQTNQS | |
| | NSYPPMSDPYMPSYYAPSIGFPYSLGEAAWSTAGDQPMPYLTT | |
| | YGQMSNGEHHYIPDGVFSQPGALGNTPPFLGQHGFNFFPGNAD | |
| | FSTWGTSGSQGQSTQSSAYSSSYGYPPSSLGRAITDGQAGFGND | |
| | TLSKVPGISSIEQGMTGLKIGGDLTAAVTKTVGTALSSSGMTSIA | |
| | TNSVPPVSSAAPKPTSWAAIARKPAKPQPKLKPKGNVGIGGSAV | |
| | PPPPIKHNMNIGTWDEKGSVVKAPPTQPVLPPQTIIQQPQPLIQPP | |
| | PLVQSQLPQQQPQPPQPQQQQGPQPQAQPHQVQPQQQQLQNR | |
| | WVAPRNRGAGFNQNNGAGSENFGLGVVPVSASPSSVEVHPVL | |
| | EKLKAINNYNPKDFDWNLKN | |
| CIRT-10 | MGIINTLQKYYCRVRGGRCAVLSCLPKEEQIGKCSTRGRKCCR | 10 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMSGDEMIFDPTMSKKKKKK | |
| | KKPFMLDEEGDTQTEETQPSETKEVEPEPTEDKDLEADEEDTRK | |
| | KDASDDLDDLNFFNQKKKKKKTKKIFDIDEAEEGVKDLKIESD | |
| | VQEPTEPEDDLDIMLGNKKKKKKNVKFPDEDEILEKDEALEDE | |
| | DNKKDDGISFSNQTGPAWAGSERDYTYEELLNRVFNIMREKNP | |
| | DMVAGEKRKFVMKPPQVVRVGTKKTSFVNFTDICKLLHRQPK | |
| | HLLAFLLAELGTSGSIDGNNQLVIKGRFQQKQIENVLRRYIKEY | |
| | VTCHTCRSPDTILQKDTRLYFLQCETCHSRCSVASIKTGFQAVT | |
| | GKRAQLRAKANGSDYKDDDDK* | |
| EIF2S2 | MSGDEMIFDPTMSKKKKKKKKPFMLDEEGDTQTEETQPSETKE | 28 |
| | VEPEPTEDKDLEADEEDTRKKDASDDLDDLNFFNQKKKKKKT | |
| | KKIFDIDEAEEGVKDLKIESDVQEPTEPEDDLDIMLGNKKKKKK | |
| | NVKFPDEDEILEKDEALEDEDNKKDDGISFSNQTGPAWAGSER | |
| | DYTYEELLNRVFNIMREKNPDMVAGEKRKFVMKPPQVVRVGT | |
| | KKTSFVNFTDICKLLHRQPKHLLAFLLAELGTSGSIDGNNQLVIK | |
| | GRFQQKQIENVLRRYIKEYVTCHTCRSPDTILQKDTRLYFLQCE | |
| | TCHSRCSVASIKTGFQAVTGKRAQLRAKAN | |
| CIRT-11 | MGIINTLQKYYCRVRGGRCAVLSCLPKEEQIGKCSTRGRKCCR | 11 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMTERRVPFSLLRGPSWDPFR | |
| | DWYPHSRLFDQAFGLPRLPEEWSQWLGGSSWPGYVRPLPPAAI | |
| | ESPAVAAPAYSRALSRQLSSGVSEIRHTADRWRVSLDVNHFAP | |
| | DELTVKTKDGVVEITGKHEERQDEHGYISRCFTRKYTLPPGVDP | |
| | TQVSSSLSPEGTLTVEAPMPKLATQSNEITIPVTFESRAQLGGPE | |
| | AAKSDETAAKLGSDYKDDDDK* | |
| HSPB1 | MTERRVPFSLLRGPSWDPFRDWYPHSRLFDQAFGLPRLPEEWS | 29 |
| | QWLGGSSWPGYVRPLPPAAIESPAVAAPAYSRALSRQLSSGVSE | |
| | IRHTADRWRVSLDVNHFAPDELTVKTKDGVVEITGKHEERQDE | |
| | HGYISRCFTRKYTLPPGVDPTQVSSSLSPEGTLTVEAPMPKLAT | |
| | QSNEITIPVTFESRAQLGGPEAAKSDETAAKL | |
| CIRT-12 | MGIINTLQKYYCRVRGGRCAVLSCLPKEEQIGKCSTRGRKCCR | 12 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMDDREDLVYQAKLAEQAER | |
| | YDEMVESMKKVAGMDVELTVEERNLLSVAYKNVIGARRASW | |
| | RIISSIEQKEENKGGEDKLKMIREYRQMVETELKLICCDILDVLD | |
| | KHLIPAANTGESKVFYYKMKGDYHRYLAEFATGNDRKEAAEN | |
| | SLVAYKAASDIAMTELPPTHPIRLGLALNFSVFYYEILNSPDRAC | |
| | RLAKAAFDDAIAELDTLSEESYKDSTLIMQLLRDNLTLWTSDM | |
| | QGDGEEQNKEALQDVEDENQGSDYKDDDDK* | |
| YWHAE | MDDREDLVYQAKLAEQAERYDEMVESMKKVAGMDVELTVEE | 30 |
| | RNLLSVAYKNVIGARRASWRIISSIEQKEENKGGEDKLKMIREY | |
| | RQMVETELKLICCDILDVLDKHLIPAANTGESKVFYYKMKGDY | |
| | HRYLAEFATGNDRKEAAENSLVAYKAASDIAMTELPPTHPIRL | |
| | GLALNFSVFYYEILNSPDRACRLAKAAFDDAIAELDTLSEESYK | |
| | DSTLIMQLLRDNLTLWTSDMQGDGEEQNKEALQDVEDENQ | |
| CIRT-13 | MGIINTLQKYYCRVRGGRCAVLSCLPKEEQIGKCSTRGRKCCR | 13 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMAEEMVEAAGEDERELAAE | |
| | MAAAFLNENLPESIFGAPKAGNGQWASVIRVMNPIQGNTLDLV | |
| | QLEQNEAAFSVAVCRFSNIGEDWYVLVGVAKDLILNPRSVAGG | |
| | FVYTYKLVNNGEKLEFLHKTPVEEVPAAIAPFQGRVLIGVGKLL | |
| | RVYDLGKKKLLRKCENKHIANYISGIQTIGHRVIVSDVQESFIW | |
| | VRYKRNENQLIIFADDTYPRWVTTASLLDYDTVAGADKFGNIC | |
| | VVRLPPNTNDEVDEDPTGNKALWDRGLLNGASQKAEVIMNYH | |
| | VGETVLSLQKTTLIPGGSESLVYTTLSGGIGILVPFTSHEDHDFFQ | |
| | HVEMHLRSEHPPLCGRDHLSFRSYYFPVKNVIDGDLCEQFNSM | |
| | EPNKQKNVSEELDRTPPEVSKKLEDIRTRYAFGSDYKDDDDK* | |
| SF3B3 | MAEEMVEAAGEDERELAAEMAAAFLNENLPESIFGAPKAGNG | 31 |
| | QWASVIRVMNPIQGNTLDLVQLEQNEAAFSVAVCRFSNIGEDW | |
| | YVLVGVAKDLILNPRSVAGGFVYTYKLVNNGEKLEFLHKTPVE | |
| | EVPAAIAPFQGRVLIGVGKLLRVYDLGKKKLLRKCENKHIANYI | |
| | SGIQTIGHRVIVSDVQESFIWVRYKRNENQLIIFADDTYPRWVTT | |
| | ASLLDYDTVAGADKFGNICVVRLPPNTNDEVDEDPTGNKALW | |
| | DRGLLNGASQKAEVIMNYHVGETVLSLQKTTLIPGGSESLVYTT | |
| | LSGGIGILVPFTSHEDHDFFQHVEMHLRSEHPPLCGRDHLSFRSY | |
| | YFPVKNVIDGDLCEQFNSMEPNKQKNVSEELDRTPPEVSKKLE | |
| | DIRTRYAF | |
| CIRT-14 | MGIINTLQKYYCRVRGGRCAVLSCLPKEEQIGKCSTRGRKCCR | 63 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMAVPETRPNHTIYINNLNEKI | |
| | KKDELKKSLYAIFSQFGQILDILVSRSLKMRGQAFVIFKEVSSAT | |
| | NALRSMQGFPFYDKPMRIQYAKTDSDIIAKMKGTFVERDRKRE | |
| | KRKPKSQETPATKKAVQGGGATPVVGAVQGPVPGMPPMTQAP | |
| | RIMHHMPGQPPYMPPPGMIPPPGLAPGQIPPGAMPPQQLMPGQ | |
| | MPPAQPLSENPPNHILFLTNLPEETNELMLSMLFNQFPGFKEVR | |
| | LVPGRHDIAFVEFDNEVQAGAARDALQGFKITQNNAMKISFAK | |
| | KGSDYKDDDDK | |
| SNRPA | MAVPETRPNHTIYINNLNEKIKKDELKKSLYAIFSQFGQILDILVS | 60 |
| | RSLKMRGQAFVIFKEVSSATNALRSMQGFPFYDKPMRIQYAKT | |
| | DSDIIAKMKGTFVERDRKREKRKPKSQETPATKKAVQGGGATP | |
| | VVGAVQGPVPGMPPMTQAPRIMHHMPGQPPYMPPPGMIPPPGL | |
| | APGQIPPGAMPPQQLMPGQMPPAQPLSENPPNHILFLTNLPEET | |
| | NELMLSMLFNQFPGFKEVRLVPGRHDIAFVEFDNEVQAGAARD | |
| | ALQGFKITQNNAMKISFAKK | |
| CIRT-15 | MGIINTLQKYYCRVRGGRCAVLSCLPKEEQIGKCSTRGRKCCR | 64 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSQLLRDNTTGSNSSLNTSLPSP | |
| | GAWPYSASDNSFTNVHSTSAKFPDYKSTWSPDPIGHNPTHLSN | |
| | KMWKNHISSRNTTPLPRPPPGLTNPKPSSPWSSTAPRSVRGWGT | |
| | QDSRLASASTWSDGGSVRPSYWLVLHNLTPQIDGSTLRTICMQ | |
| | HGPLLTFHLNLTQGTALIRYSTKQEAAKAQTALHMCVLGNTTI | |
| | LAEFATDDEVSRFLAQAQPPTPAATPSAPAAGWQSLETGQNQS | |
| | DPVGPALNLFGGSTGLGQWSSSAGGSSGADLAGASLWGPPNYS | |
| | SSLWGVPTVEDPHRMGSPAPLLPGDLLGGGSDSIGSDYKDDDD | |
| | K* | |
| TNRC6B | QLLRDNTTGSNSSLNTSLPSPGAWPYSASDNSFTNVHSTSAKFP | 61 |
| | DYKSTWSPDPIGHNPTHLSNKMWKNHISSRNTTPLPRPPPGLTN | |
| | PKPSSPWSSTAPRSVRGWGTQDSRLASASTWSDGGSVRPSYWL | |
| | VLHNLTPQIDGSTLRTICMQHGPLLTFHLNLTQGTALIRYSTKQE | |
| | AAKAQTALHMCVLGNTTILAEFATDDEVSRFLAQAQPPTPAAT | |
| | PSAPAAGWQSLETGQNQSDPVGPALNLFGGSTGLGQWSSSAG | |
| | GSSGADLAGASLWGPPNYSSSLWGVPTVEDPHRMGSPAPLLPG | |
| | DLLGGGSDSI | |
| CIRT-16 | MGIINTLQKYYCRVRGGRCAVLSCLPKEEQIGKCSTRGRKCCR | 65 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMSAANPETPNSTISREASTQS | |
| | SSAAASQGWVLPEGKIVPNTVFVGGIDARMDETEIGSCFGRYGS | |
| | VKEVKIITNRTGVSKGYGFVSFVNDVDVQKIVGSQIHFHGKKLK | |
| | LGPAIRKQKLCARHVQPRPLVVNPPPPPQFQNVWRNPNTETYL | |
| | QPQITPNPVTQHVQSAANPETPNSTISREASTQSSSAAASQGWV | |
| | LPEGKIVPNTVFVGGIDARMDETEIGSCFGRYGSVKEVKIITNRT | |
| | GVSKGYGFVSFVNDVDVQKIVGSQIHFHGKKLKLGPAIRKQKL | |
| | CARHVQPRPLVVNPPPPPQFQNVWRNPNTETYLQPQITPNPVTQ | |
| | HVQAYSAYPHSPGQVITGCQLLVYNYQEYPTYPDSAFQVTTGY | |
| | QLPVYNYQPFPAYPRSPFQVTAGYQLPVYNYQAFPAYPNSPFQ | |
| | VATGYQFPVYNYQPFPAYPSSPFQVTAGYQLPVYNYQAFPAYP | |
| | NSPFQVATGYQFPVYNYQAFPAYPNSPVQVTTGYQLPVYNYQ | |
| | AFPAYPSSPFQVTTGYQLPVYNYQAFPAYPNSAVQVTTGYQFH | |
| | VYNYQMPPQCPVGEQRRNLWTEAYKWWYLVCLIQRRDGSDY | |
| | KDDDDK* | |
| DAZ4 | MSAANPETPNSTISREASTQSSSAAASQGWVLPEGKIVPNTVFV | 62 |
| | GGIDARMDETEIGSCFGRYGSVKEVKIITNRTGVSKGYGFVSFV | |
| | NDVDVQKIVGSQIHFHGKKLKLGPAIRKQKLCARHVQPRPLVV | |
| | NPPPPPQFQNVWRNPNTETYLQPQITPNPVTQHVQSAANPETPN | |
| | STISREASTQSSSAAASQGWVLPEGKIVPNTVFVGGIDARMDET | |
| | EIGSCFGRYGSVKEVKIITNRTGVSKGYGFVSFVNDVDVQKIVG | |
| | SQIHFHGKKLKLGPAIRKQKLCARHVQPRPLVVNPPPPPQFQNV | |
| | WRNPNTETYLQPQITPNPVTQHVQAYSAYPHSPGQVITGCQLL | |
| | VYNYQEYPTYPDSAFQVTTGYQLPVYNYQPFPAYPRSPFQVTA | |
| | GYQLPVYNYQAFPAYPNSPFQVATGYQFPVYNYQPFPAYPSSP | |
| | FQVTAGYQLPVYNYQAFPAYPNSPFQVATGYQFPVYNYQAFP | |
| | AYPNSPVQVTTGYQLPVYNYQAFPAYPSSPFQVTTGYQLPVYN | |
| | YQAFPAYPNSAVQVTTGYQFHVYNYQMPPQCPVGEQRRNLWT | |
| | EAYKWWYLVCLIQRRD | |
| CIRT-17 | MGIINTLQKYYCRVRGGRCAVLSCLPKEEQIGKCSTRGRKCCR | 66 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMSSEAETQQPPAAPPAAPALS | |
| | AADTKPGTTGSGAGSGGPGGLTSAAPAGGDKKVIATKVLGTV | |
| | KWFNVRNGYGFINRNDTKEDVFVHQTAIKKNNPRKYLRSVGD | |
| | GETVEFDVVEGEKGAEAANVTGPGGVPVQGSKYAADRNHYRR | |
| | YPRRRGPPRNYQQNYQNSESGEKNEGSESAPEGQAQQRRPYRR | |
| | RRFPPYYMRRPYGRRPQYSNPPVQGEVMEGADNQGAGEQGRP | |
| | VRQNMYRGYRPRFRRGPPRQRQPREDGNEEDKENQGDETQGQ | |
| | QPPQRRYRRNFNYRRRRPENPKPQDGKETKAADPPAENSSAPE | |
| | AEQGGAEGSDYKDDDDK* | |
| YB-1 | MSSEAETQQPPAAPPAAPALSAADTKPGTTGSGAGSGGPGGLT | 69 |
| | SAAPAGGDKKVIATKVLGTVKWFNVRNGYGFINRNDTKEDVF | |
| | VHQTAIKKNNPRKYLRSVGDGETVEFDVVEGEKGAEAANVTG | |
| | PGGVPVQGSKYAADRNHYRRYPRRRGPPRNYQQNYQNSESGE | |
| | KNEGSESAPEGQAQQRRPYRRRRFPPYYMRRPYGRRPQYSNPP | |
| | VQGEVMEGADNQGAGEQGRPVRQNMYRGYRPRFRRGPPRQR | |
| | QPREDGNEEDKENQGDETQGQQPPQRRYRRNFNYRRRRPENP | |
| | KPQDGKETKAADPPAENSSAPEAEQGGAE | |
| CIRT-18 | MGIINTLQKYYCRVRGGRCAVLSCLPKEEQIGKCSTRGRKCCR | 67 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMSSEAETQQPPAAPPAAPALS | |
| | AADTKPGTTGSGAGSGGPGGLTSAAPAGGDKKVIATKVLGTV | |
| | KWFNVRNGYGFINRNDTKEDVFVHQTAIKKNNPRKYLRSVGD | |
| | GETVEFDVVEGEKGAEAANVTGPGGVPVQGSKYAADRNHYRR | |
| | YPRRRGPPRNYQQNYQNSESGEKNEGSESAPEGQAQQRRPYRR | |
| | RRFPPYYMRRPYGRRPQYSNPPVQGEVMEGADNQGAGEQGRP | |
| | VRQNMYRGYRPRFRRGPPRQRQPREDGNEEDKENQGDETQGQ | |
| | QPPQRRYRRNFNYRRRRPENPKPQDGKETKAADPPAENSSAPE | |
| | AEQGGAEGGSGGSGGSGGSGGSGGSLHLDQTPSRQPIPSEGLQL | |
| | HLPQVLADAVSRLVLGKFGDLTDNESSPHARRKVLAGVVMTT | |
| | GTDVKDAKVISVSTGTKCINGEYMSDRGLALNDCHAEIISRRSL | |
| | LRFLYTQLELYLNNKDDQKRSIFQKSERGGFRLKENVQFHLYIS | |
| | TSPCGDARIFSPHEPILEEPADRHPNRKARGQLRTKIESGQGTIPV | |
| | RSNASIQTWDGVLQGERLLTMSCSDKIARWNVVGIQGSLLSIFV | |
| | EPIYFSSIILGSLYHGDHLSRAMYQRISNIEDLPPLYTLNKPLLSGI | |
| | SNAEARQPGKAPNFSVNWTVGDSAIEVINATTGKDELGRASRL | |
| | CKHALYCRWMRVHGKVPSHLLRSKITKPNVYHESKLAAKEYQ | |
| | AAKARLFTAFIKAGLGAWVEKPTEQDQFSLTPGSDYKDDDDK* | |
| YB-1- | MSSEAETQQPPAAPPAAPALSAADTKPGTTGSGAGSGGPGGLT | 72 |
| ADAR2 | SAAPAGGDKKVIATKVLGTVKWFNVRNGYGFINRNDTKEDVF | |
| (E488Q) | VHQTAIKKNNPRKYLRSVGDGETVEFDVVEGEKGAEAANVTG | |
| | PGGVPVQGSKYAADRNHYRRYPRRRGPPRNYQQNYQNSESGE | |
| | KNEGSESAPEGQAQQRRPYRRRRFPPYYMRRPYGRRPQYSNPP | |
| | VQGEVMEGADNQGAGEQGRPVRQNMYRGYRPRFRRGPPRQR | |
| | QPREDGNEEDKENQGDETQGQQPPQRRYRRNFNYRRRRPENP | |
| | KPQDGKETKAADPPAENSSAPEAEQGGAEGGSGGSGGSGGSG | |
| | GSGGSLHLDQTPSRQPIPSEGLQLHLPQVLADAVSRLVLGKFGD | |
| | LTDNFSSPHARRKVLAGVVMTTGTDVKDAKVISVSTGTKCING | |
| | EYMSDRGLALNDCHAEIISRRSLLRFLYTQLELYLNNKDDQKRS | |
| | IFQKSERGGFRLKENVQFHLYISTSPCGDARIFSPHEPILEEPADR | |
| | HPNRKARGQLRTKIESGQGTIPVRSNASIQTWDGVLQGERLLT | |
| | MSCSDKIARWNVVGIQGSLLSIFVEPIYFSSIILGSLYHGDHLSRA | |
| | MYQRISNIEDLPPLYTLNKPLLSGISNAEARQPGKAPNFSVNWT | |
| | VGDSAIEVINATTGKDELGRASRLCKHALYCRWMRVHGKVPS | |
| | HLLRSKITKPNVYHESKLAAKEYQAAKARLFTAFIKAGLGAWV | |
| | EKPTEQDQFSLTP | |
| ADAR2 | LHLDQTPSRQPIPSEGLQLHLPQVLADAVSRLVLGKFGDLTDNF | 70 |
| (E488Q) | SSPHARRKVLAGVVMTTGTDVKDAKVISVSTGTKCINGEYMSD | |
| | RGLALNDCHAEIISRRSLLRFLYTQLELYLNNKDDQKRSIFQKSE | |
| | RGGFRLKENVQFHLYISTSPCGDARIFSPHEPILEEPADRHPNRK | |
| | ARGQLRTKIESGQGTIPVRSNASIQTWDGVLQGERLLTMSCSDK | |
| | IARWNVVGIQGSLLSIFVEPIYFSSIILGSLYHGDHLSRAMYQRIS | |
| | NIEDLPPLYTLNKPLLSGISNAEARQPGKAPNFSVNWTVGDSAIE | |
| | VINATTGKDELGRASRLCKHALYCRWMRVHGKVPSHLLRSKIT | |
| | KPNVYHESKLAAKEYQAAKARLFTAFIKAGLGAWVEKPTEQD | |
| | QFSLTP | |
| CIRT-19 | MGIINTLQKYYCRVRGGRCAVLSCLPKEEQIGKCSTRGRKCCR | 68 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMSSEAETQQPPAAPPAAPALS | |
| | AADTKPGTTGSGAGSGGPGGLTSAAPAGGDKKVIATKVLGTV | |
| | KWFNVRNGYGFINRNDTKEDVFVHQTAIKKNNPRKYLRSVGD | |
| | GETVEFDVVEGEKGAEAANVTGPGGVPVQGSKYAADRNHYRR | |
| | YPRRRGPPRNYQQNYQNSESGEKNEGSESAPEGQAQQRRPYRR | |
| | RRFPPYYMRRPYGRRPQYSNPPVQGEVMEGADNQGAGEQGRP | |
| | VRQNMYRGYRPRFRRGPPRQRQPREDGNEEDKENQGDETQGQ | |
| | QPPQRRYRRNFNYRRRRPENPKPQDGKETKAADPPAENSSAPE | |
| | AEQGGAEGGSGGSGGSGGSGGSGGSLHLDQTPSRQPIPSEGLQL | |
| | HLPQVLADAVSRLVLGKFGDLTDNFSSPHARRKVLAGVVMTT | |
| | GTDVKDAKVISVSTGTKCINGEYMSDRGLALNDCHAEIISRRSL | |
| | LRFLYTQLELYLNNKDDQKRSIFQKSERGGFRLKENVQFHLYIS | |
| | TSPCGDARIFSPHEPILEEPADRHPNRKARGQLRTKIESGEGTIPV | |
| | RSNASIQTWDGVLQGERLLTMSCSDKIARWNVVGIQGSLLSIFV | |
| | EPIYFSSIILGSLYHGDHLSRAMYQRISNIEDLPPLYTLNKPLLSGI | |
| | SNAEARQPGKAPNFSVNWTVGDSAIEVINATTGKDELGRASRL | |
| | CKHALYCRWMRVHGKVPSHLLRSKITKPNVYHESKLAAKEYQ | |
| | AAKARLFTAFIKAGLGAWVEKPTEQDQFSLTP | |
| | GSDYKDDDDK* | |
| YB-1- | MSSEAETQQPPAAPPAAPALSAADTKPGTTGSGAGSGGPGGLT | 73 |
| ADAR2 | SAAPAGGDKKVIATKVLGTVKWFNVRNGYGFINRNDTKEDVF | |
| (wt) | VHQTAIKKNNPRKYLRSVGDGETVEFDVVEGEKGAEAANVTG | |
| | PGGVPVQGSKYAADRNHYRRYPRRRGPPRNYQQNYQNSESGE | |
| | KNEGSESAPEGQAQQRRPYRRRRFPPYYMRRPYGRRPQYSNPP | |
| | VQGEVMEGADNQGAGEQGRPVRQNMYRGYRPRFRRGPPRQR | |
| | QPREDGNEEDKENQGDETQGQQPPQRRYRRNFNYRRRRPENP | |
| | KPQDGKETKAADPPAENSSAPEAEQGGAEGGSGGSGGSGGSG | |
| | GSGGSLHLDQTPSRQPIPSEGLQLHLPQVLADAVSRLVLGKFGD | |
| | LTDNFSSPHARRKVLAGVVMTTGTDVKDAKVISVSTGTKCING | |
| | EYMSDRGLALNDCHAEIISRRSLLRFLYTQLELYLNNKDDQKRS | |
| | IFQKSERGGFRLKENVQFHLYISTSPCGDARIFSPHEPILEEPADR | |
| | HPNRKARGQLRTKIESGEGTIPVRSNASIQTWDGVLQGERLLTM | |
| | SCSDKIARWNVVGIQGSLLSIFVEPIYFSSIILGSLYHGDHLSRAM | |
| | YQRISNIEDLPPLYTLNKPLLSGISNAEARQPGKAPNFSVNWTV | |
| | GDSAIEVINATTGKDELGRASRLCKHALYCRWMRVHGKVPSH | |
| | LLRSKITKPNVYHESKLAAKEYQAAKARLFTAFIKAGLGAWVE | |
| | KPTEQDQFSLTP | |
| ADAR2 | LHLDQTPSRQPIPSEGLQLHLPQVLADAVSRLVLGKFGDLTDNF | 71 |
| (wt) | SSPHARRKVLAGVVMTTGTDVKDAKVISVSTGTKCINGEYMSD | |
| | RGLALNDCHAEIISRRSLLRFLYTQLELYLNNKDDQKRSIFQKSE | |
| | RGGFRLKENVQFHLYISTSPCGDARIFSPHEPILEEPADRHPNRK | |
| | ARGQLRTKIESGEGTIPVRSNASIQTWDGVLQGERLLTMSCSDK | |
| | IARWNVVGIQGSLLSIFVEPIYFSSIILGSLYHGDHLSRAMYQRIS | |
| | NIEDLPPLYTLNKPLLSGISNAEARQPGKAPNFSVNWTVGDSAIE | |
| | VINATTGKDELGRASRLCKHALYCRWMRVHGKVPSHLLRSKIT | |
| | KPNVYHESKLAAKEYQAAKARLFTAFIKAGLGAWVEKPTEQD | |
| | QFSLTP | |
| CIRT-20 | MGIINTLQKYYCRVRGGRCAVLSCLPKEEQIGKCSTRGRKCCR | 77 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMAELTVEVRGSNGAFYKGFI | |
| | KDVHEDSLTVVFENNWQPERQVPFNEVRLPPPPDIKKEISEGDE | |
| | VEVYSRANDQEPCGWWLAKVRMMKGEFYVIEYAACDATYNE | |
| | IVTFERLRPVNQNKTVKKNTFFKCTVDVPEDLREACANENAHK | |
| | DFKKAVGACRIFYHPETTQLMILSASEATVKRVNILSDMHLRSI | |
| | RTKLMLMSRNEEATKHLECTKQLAAAFHEEFVVREDLMGLAI | |
| | GTHGSNIQQARKVPGVTAIELDEDTGTFRIYGESADAVKKARGF | |
| | LEFVEDFIQVPRNLVGKVIGKNGKVIQEIVDKSGVVRVRIEGDN | |
| | ENKLPREDGMVPFVFVGTKESIGNVQVLLEYHIAYLKEVEQLR | |
| | MERLQIDEQLRQIGSRSYSGRGRGRRGPNYTSGYGTNSELSNPS | |
| | ETESERKDELSDWSLAGEDDRDSRHQRDSRRRPGGRGRSVSGG | |
| | RGRGGPRGGKSSISSVLKDPDSNPYSLLDNTESDQTADTDASES | |
| | HHSTNRRRRSRRRRTDEDAVLMDGMTESDTASVNENGLGKRC | |
| | DGSDYKDDDDK | |
| FXR1 | MAELTVEVRGSNGAFYKGFIKDVHEDSLTVVFENNWQPERQV | 74 |
| | PFNEVRLPPPPDIKKEISEGDEVEVYSRANDQEPCGWWLAKVR | |
| | MMKGEFYVIEYAACDATYNEIVTFERLRPVNQNKTVKKNTFFK | |
| | CTVDVPEDLREACANENAHKDFKKAVGACRIFYHPETTQLMIL | |
| | SASEATVKRVNILSDMHLRSIRTKLMLMSRNEEATKHLECTKQ | |
| | LAAAFHEEFVVREDLMGLAIGTHGSNIQQARKVPGVTAIELDE | |
| | DTGTFRIYGESADAVKKARGFLEFVEDFIQVPRNLVGKVIGKNG | |
| | KVIQEIVDKSGVVRVRIEGDNENKLPREDGMVPFVFVGTKESIG | |
| | NVQVLLEYHIAYLKEVEQLRMERLQIDEQLRQIGSRSYSGRGRG | |
| | RRGPNYTSGYGTNSELSNPSETESERKDELSDWSLAGEDDRDS | |
| | RHQRDSRRRPGGRGRSVSGGRGRGGPRGGKSSISSVLKDPDSN | |
| | PYSLLDNTESDQTADTDASESHHSTNRRRRSRRRRTDEDAVLM | |
| | DGMTESDTASVNENGLGKRCD | |
| CIRT-21 | MGIINTLQKYYCRVRGGRCAVLSCLPKEEQIGKCSTRGRKCCR | 78 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMADRAPRQPTSLGSSPQRCG | |
| | SPPSAPPPPPDRGERIEDCLAPLCPPVVGGRGGCALSRFLPVDLS | |
| | LLRSSRTAAVELVAGGVCRSPSQENGSDYKDDDDK | |
| APPLE | MADRAPRQPTSLGSSPQRCGSPPSAPPPPPDRGERIEDCLAPLCP | 75 |
| | PVVGGRGGCALSRFLPVDLSLLRSSRTAAVELVAGGVCRSPSQ | |
| | EN | |
| CIRT-22 | MGIINTLQKYYCRVRGGRCAVLSCLPKEEQIGKCSTRGRKCCR | 79 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMNNKFDALKDDDSGDHDQN | |
| | EENSTQKDGEKEKTERDKNQSSSKRKAVVPGPAEHPLQYNYTF | |
| | WYSRRTPGRPTSSQSYEQNIKQIGTFASVEQFWRFYSHMVRPG | |
| | DLTGHSDFHLFKEGIKPMWEDDANKNGGKWIIRLRKGLASRC | |
| | WENLILAMLGEQFMVGEEICGAVVSVRFQEDIISIWNKTASDQA | |
| | TTARIRDTLRRVLNLPPNTIMEYKTHTDSIKMPGRLGPQRLLFQ | |
| | NLWKPRLNVPGSDYKDDDDK | |
| eIF4E2 | MNNKFDALKDDDSGDHDQNEENSTQKDGEKEKTERDKNQSSS | 76 |
| | KRKAVVPGPAEHPLQYNYTFWYSRRTPGRPTSSQSYEQNIKQIG | |
| | TFASVEQFWRFYSHMVRPGDLTGHSDFHLFKEGIKPMWEDDA | |
| | NKNGGKWIIRLRKGLASRCWENLILAMLGEQFMVGEEICGAVV | |
| | SVRFQEDIISIWNKTASDQATTARIRDTLRRVLNLPPNTIMEYKT | |
| | HTDSIKMPGRLGPQRLLFQNLWKPRLNVP | |

### VIII. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: KNOCKDOWN OF PMP22 TRANSCRIPT IN NIH/3T3 CELLS BY CIRTS-Pin AND CIRTS-Y2

NIH-3T3 mouse fibroblasts were plated on 96-well plates and transfected at 70% confluency 24 hours later. 125 ng of the indicated gRNA-CIRTS expression plasmids was transfected per well using Lipofectamine LTX and Opti-MEM I Reduced Serum Medium (ThermoFisher Scientific) according to the manufacture's protocol. After 48 hours, total RNA was harvested and isolated using the RNeasy Mini Kit (QIAGEN). After RNA isolation, RNA was reverse transcribed to cDNA using the PrimeScript RT Reagent Kit (TaKaRa). All qPCR reactions were run at 20 μL volumes with at least three biological replicates using PowerUp SYBR Green Master Mix (Applied Biosystems) and amplified on a LightCycler 96 Instrument (Roche). All qPCR primers used were designed on NCBI Primer BLAST and validated by no measured amplification in the absence of cDNA, a sharp, single-product melting temperature, and an amplification efficiency within 95-105%.

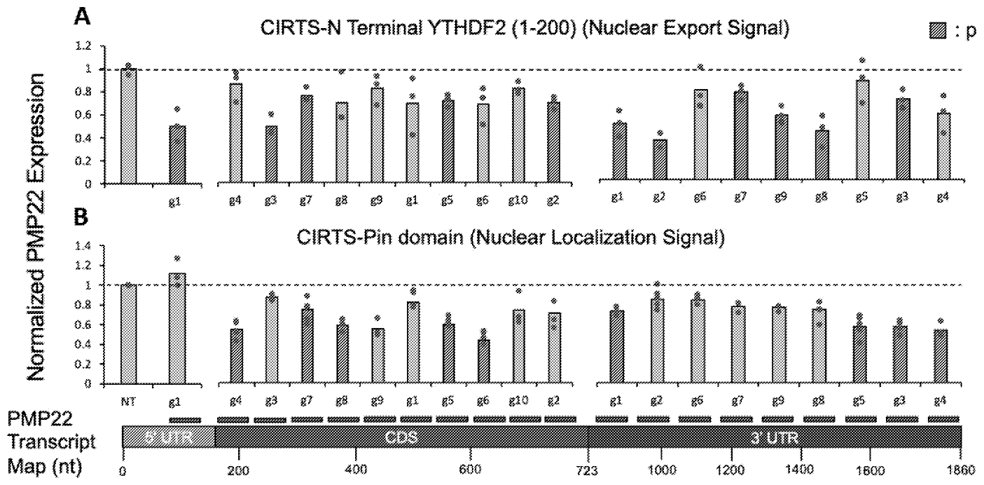

Expression levels were calculated using the housekeeping control gene (GAPDH) cycle threshold (Ct) value and the gene of interest Ct value. The relative expression level of PMP22 was determined by 2dCt, where dCt=Ct(PMP22)−Ct(GAPDH). Relative expression level for targeted gene was obtained upon normalizing the targeted gene expression level of cells experiments treated with the on-target gRNA to those treated by the nontargeting (NT) gRNA. The qPCR primers used in FIG. 1 are shown in the table below:

| | | SEQ |
|---|---|---|
| Sequences of qPCR primers used in FIG. 1 | | ID NO: |
| mPMP22 Primer 1 | cgtccaacactgctactcctca | 83 |
| mPMP22 Primer 2 | gcctttggtgagagtgaagagc | 84 |
| mGAPDH Primer 1 | CATCACTGCCACCCAGAAGACTG | 85 |
| mGAPDH Primer 2 | ATGCCAGTGAGCTTCCCGTTCAG | 86 |

### Example 2: PMP22 ACTIVATION WITH eIF4E AND Y1-BASED CIRTS ACTIVATORS

SK-MEL-28 cells were plated in a 12-well plate and transfected 24 hours later using Lipofectamine LTX according to the manufacturer's protocol. Each well was transfected with a CIRTS (Bdef-TBP) expression plasmid containing either Y1 or EIF4E as the effector domain, along with a gRNA expression plasmid which was either nontargeting (NT) or complementary to the PMP22 mRNA. Protein was isolated from the SK-MEL-28 cells 48 hours later by incubation with RIPA buffer and protease inhibitors.

For western blotting, 22.5 μg of total protein was loaded for each condition then PMP22 and GAPDH levels were detected by incubation with anti-PMP22-HRP or anti-GAPDH-HRP antibody followed by chemiluminescent detection. When compared to the GAPDH loading control, transfection with Bdef-TBP-EIF4E and either g15, g17, or g1 appears to increase PMP22 protein present compared to the NT gRNA. Transfection with Bdef-TBP-Y1 and either g15 or g17 may also demonstrate detectable increases in PMP22 protein levels. All other conditions show PMP22 levels comparable to the NT negative control.

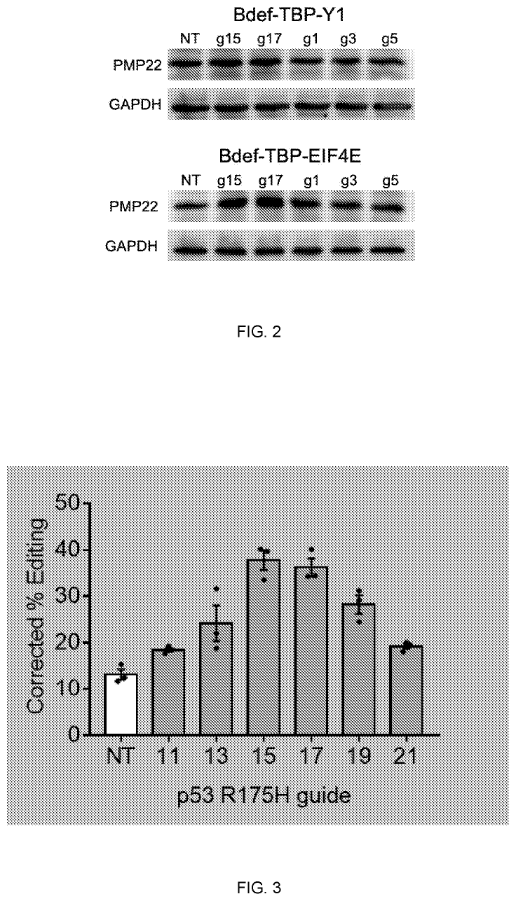

As shown in FIG. 2, g15 (targets 5′UTR) and g17 (targets 3′UTR) activate PMP22 expression, especially with an eIF4E-based CIRTS system.

### Example 3: P53 EDITING WITH CIRTS-ADAR EDITOR

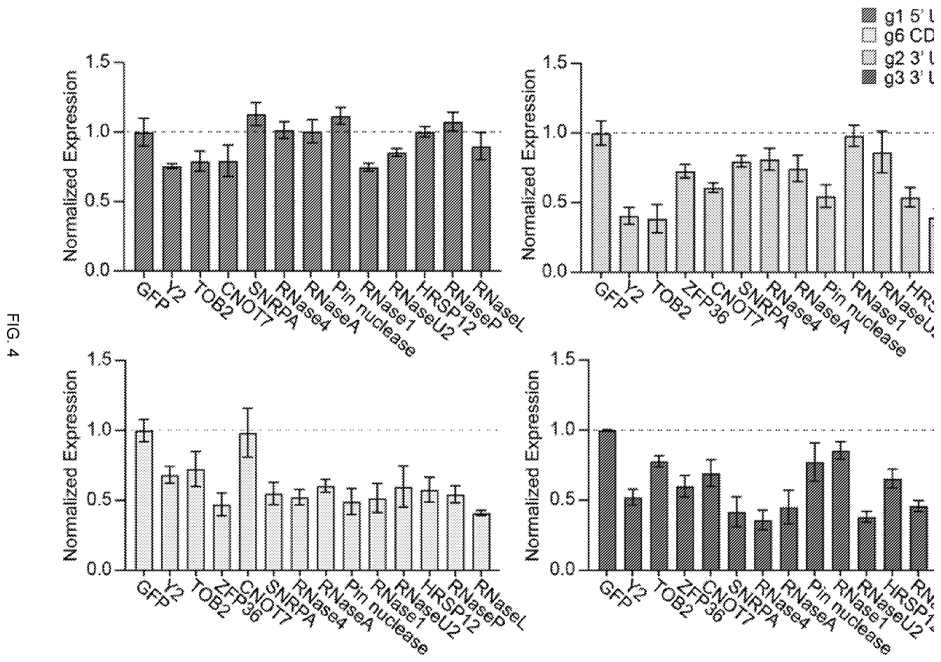

HEK293 cells were transfected using Lipofectamine 2000 with CIRTS-ADAR expression plasmid (125 ng), a gRNA expression plasmid (100 ng), and a P53 R175H reporter (12 ng). The gRNA expression plasmids expressed either a non-targeting control guide or one of six editing guides tiling the p53 R175 position. Each of the editing guides were designed such that the mismatch position, which directs the intended editing of p53 R175H, occurred at 11, 13, 15, 17, 19, or 21 nt from the 5′ end of the gRNA sequence. Each gRNA included two TAR hairpins. Editing efficiency of the guides to generate p53 R175H edited mRNA was monitored by Sanger sequence of RT-qPCR products. FIG. 3 demonstrates that the CIRTS-ADAR editor delivered to cells along with gRNAs can edit p53 mismatch sites and the highest editing is observed when the mismatch occurs 15 nucleotides from the 5′ end of the gRNA sequence.

The gRNAs used in FIGS. 1-2 are shown in the tables below.

FIG. 1 mouse PMP22 targeting sequences

| gRNA | | SEQ |
|---|---|---|
| Localization | | ID |
| and Number | gRNA Sequence | NO: |
| Non- | TGACAGCCCACATGGCATTCCACTTATCACTGGCATCCTT | 87 |
| targeting | | |
| guide (NT) | | |

5′ Untranslated Region (UTR)

| | | |
|---|---|---|
| g1 | GTGGCTGGGAGTTGGGCTCGGGATCAGAGGAGCGGTGCCT | 88 |

Coding Sequence (CDS)

| | | |
|---|---|---|
| g1 | CAGAAGAACAGGAACAGAGCCAGGACGCTGAAGATGACA | 89 |
| | G | |
| g2 | ATCACATAGATGATACCACTGAGGAGGGCTAGGGGAAAGG | 90 |
| g3 | GAGACGAAGAGCAACACTAGCACCGCGATGTGCAGGAAC | 91 |
| | A | |
| g4 | CGATGTGCAGGAACAGGATCCCCAACAAGAGTAGGAGCAT | 92 |
| g5 | AATCCAGTGATGTAAAACCGGCCGCCTTTGGTGAGAGTGA | 93 |
| g6 | ATGCCACTCACTGTGCCTCACTGTGTAGATGGCCGCTGCA | 94 |
| g7 | TCAGTCGTGTGTCCATTGCCCACGAGCCATTGGCTGACGA | 95 |
| g8 | AGACTGCAGCCATTCGCTCACTGATGAGGAGTAGCAGTGT | 96 |
| g9 | CTGAAGATGACAGACAGGATCATGGTGGCCTGGACAGACT | 97 |
| g10 | GCGAAGCCATAGGAGTAGTCAGTGTTGACATGCCACTCAC | 98 |

3′ UTR

| | | |
|---|---|---|
| g1 | CGCGCTCAGAGCCTAGACGGACGGTGCGTCGTCGGGCGCC | 99 |
| g2 | CCACAGGGAGGGAGGAAGGAAACCAGAGAACAAAACCAA | 100 |
| | C | |
| g3 | TTGGTGGCCAATACAAGTCATCGCCAGACAGTCCTTGGAG | 101 |
| g4 | CAGTTGGTATAAAATCAGAGAAAGCAAGCAAAAACAAGA | 102 |
| | A | |
| g5 | GATTTATTTATTGTAGTAATAACAACAGCCTAGCTAGGTA | 103 |
| g6 | GATATTATATACATCTTCAATCAACAGCAATCCCCACTCA | 104 |
| g7 | CAAAACAAAGATACTGAGCTTGGTTATAGTGTTAGGATGT | 105 |
| g8 | CTTGTCTCTGACTTCTCATTTAAACGTGTGGCCCCAGTAC | 106 |
| g9 | TGCACTGGGTCACCCATAGTGTAAAGGTCTTCAACTGTCT | 107 |

FIG. 2 human PMP22 targeting sequences

| gRNA | | SEQ |
|---|---|---|
| Localization | | ID |
| and Number | gRNA Sequence | NO: |
| Non- | TGACAGCCCACATGGCATTCCACTTATCACTGGCATCCTT | 87 |
| targeting | | |
| guide (NT) | | |

5′ UTR

| | | |
|---|---|---|
| g1 | GCTCAGCGGAGTTTCTGCCCGGCCAAACAGCGTAACCCCT | 108 |
| g15 | CTGGCGGCAAGTTCTGCTCAGCGGAGTTTCTGCCCGGCCA | 109 |

Coding Sequence (CDS)

| | | |
|---|---|---|
| g3 | GGGTGAAGAGTTGGCAGAAGAACAGGAACAGAGACAGAA | 110 |
| | T | |

3′ UTR

| | | |
|---|---|---|
| g5 | TTTTCCCTTCCTCCCTTCCCTATGTACGCTCAGAGCCTCAG | 111 |
| g17 | CCTATGTACGCTCAGAGCCTCAGACAGACCGTCTGGGCGC | 112 |

| | | |
|---|---|---|
| Description | Sequences | SEQ ID NO: |

CIRTS used in FIG 2.

| | | |
|---|---|---|
| Bdef-TBP- | MGIINTLQKYYCRVRGGRCAVLSCLPKEEQIGKCSTRGRKCCRRK | 80 |
| Y1 (N- | KGGSGGSGGSGGSGGSGGSMAVPETRPNHTIYINNLNSKIKKDEL | |
| terminal | KKSLYAIFSQFGQILDILVPRQRTPRGQAFVIFKEVSSATNALRSM | |
| truncation) | QGFPFYDKPMRIQYAKTDKRIPAKMKGTFVGSLQLPPLERLTLGG | |
| | SGGSGGSGGSGGSGGSMSATSVDTQRTKGQDNKVQNGSLHQKD | |
| | TVHDNDFEPYLTGQSNQSNSYPSMSDPYLSSYYPPSIGFPYSLNEA | |
| | PWSTAGDPPIPYLTTYGQLSNGDHHFMHDAVFGQPGGLGNNIYQ | |
| | HRFNFFPENPAFSAWGTSGSQGQQTQSSAYGSSYTYPPSSLGGTV | |
| | VDGQPGFHSDTLSKAPGMNSLEQGMVGLKIGDVSSSAVKTVGSV | |
| | VSSVALTGVLSGNGGTNVNMPVSKPTSWAAIASKPAKPQPKMKT | |
| | KSGPVMGGGLPPPPIKHNMDIGTWDNKGPVPKAPVPQQAPSPQA | |
| | APQPQQVAQPLPAQPPALAQPQYQSPQQPPQTRWVAPRNRNAAF | |
| | GQSGGAGSDSNSPGNVQPNSAPSVESGSDYKDDDDK | |
| Bdef-TBP- | MGIINTLQKYYCRVRGGRCAVLSCLPKEEQIGKCSTRGRKCCRRK | 81 |
| EIF4E | KGGSGGSGGSGGSGGSGGSMAVPETRPNHTIYINNLNSKIKKDEL | |
| | KKSLYAIFSQFGQILDILVPRQRTPRGQAFVIFKEVSSATNALRSM | |
| | QGFPFYDKPMRIQYAKTDKRIPAKMKGTFVGSLQLPPLERLTLGG | |
| | SGGSGGSGGSGGSGGSMATVEPETTPTPNPPTTEEEKTESNQEVA | |
| | NPEHYIKHPLQNRWALWFFKNDKSKTWQANLRLISKFDTVEDFW | |
| | ALYNHIQLSSNLMPGCDYSLFKDGIEPMWEDEKNKRGGRWLITL | |
| | NKQQRRSDLDRFWLETLLCLIGESFDDYSDDVCGAVVNVRAKGD | |
| | KIAIWTTECENREAVTHIGRVYKERLGLPPKIVIGYQSHADTATKS | |
| | GSTTKNRFVVGSDYKDDDDK | |

CIRTS used in FIG 3.

| | | |
|---|---|---|
| CIRTS- | MGIINTLQKYYCRVRGGRCAVLSCLPKEEQIGKCSTRGRKCCRRK | 82 |
| ADAR | KGGSGGSGGSGGSGGSGGSMAVPETRPNHTIYINNLNSKIKKDEL | |
| (Bdef-TBP- | KKSLYAIFSQFGQILDILVPRQRTPRGQAFVIFKEVSSATNALRSM | |
| ADAR) | QGFPFYDKPMRIQYAKTDKRIPAKMKGTFVGGSGGSGGSGGSGG | |
| | SGGSLHLDQTPSRQPIPSEGLQLHLPQVLADAVSRLVLGKFGDLT | |
| | DNFSSPHARRKVLAGVVMTTGTDVKDAKVISVSTGTKCINGEYM | |
| | SDRGLALNDCHAEIISRRSLLRFLYTQLELYLNNKDDQKRSIFQKS | |
| | ERGGFRLKENVQFHLYISTSPCGDARIFSPHEPILEEPADRHPNRKA | |
| | RGQLRTKIESGEGTIPVRSNASIQTWDGVLQGERLLTMSCSDKIAR | |
| | WNVVGIQGSLLSIFVEPIYFSSIILGSLYHGDHLSRAMYQRISNIEDL | |
| | PPLYTLNKPLLSGISNAEARQPGKAPNFSVNWTVGDSAIEVINATT | |
| | GKDELGRASRLCKHALYCRWMRVHGKVPSHLLRSKITKPNVYHE | |
| | SKLAAKEYQAAKARLFTAFIKAGLGAWVEKPTEQDQFSLTPKRP | |
| | AATKKAGQAKKKKGSDYKDDDDK | |

### Example 4: RNA Degraders

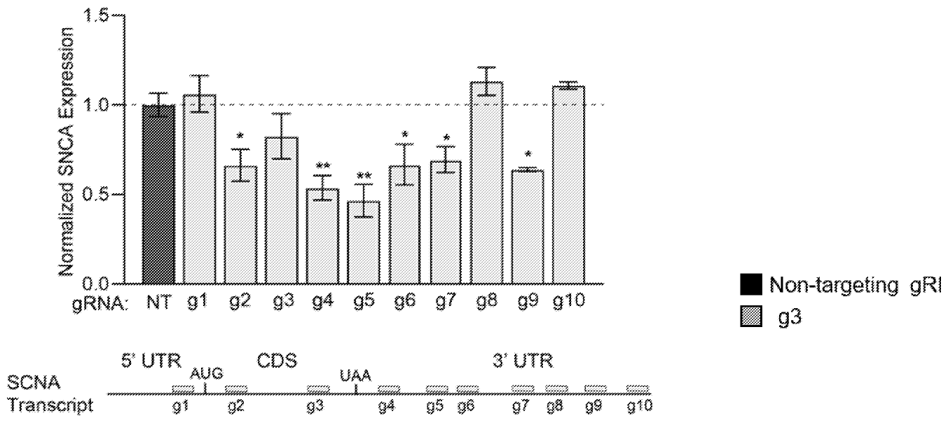

### A. FIG. 4 Methods and Results

Methods: See qPCR and transfection methods described in Example 1.

Results: Several novel CIRTS degraders were tested in NIH-3T3 cells along with gRNAs validated in Example 1 that land in the coding sequence (g6 CDS), 3′ UTR (g2 and g3 3′ UTR), and 5′ UTR (g1 5′ UTR). PMP22 transcript degradation was measured by RT-qPCR as described in Example 1. These experiments demonstrated PMP22 transcript degradation from each CIRTS tested with PMP22 expression levels depending on the effector and gRNA pair deployed. Additionally, we have targeted these degraders to the 5′ UTR as well but found this gRNA landing site was generally suboptimal for CIRTS-mediated degradation.

TABLE
Effectors

| | | SEQ |
|---|---|---|
| | | ID |
| Description | Sequence | NO: |
| GFP | VSKGEELFTGVVPILVELDGDVNGHKFSVSGEGEGDATYGKLT | 113 |
| | LKFICTTGKLPVPWPTLVTTLTYGVQCFSRYPDHMKQHDFFKS | |
| | AMPEGYVQERTIFFKDDGNYKTRAEVKFEGDTLVNRIELKGIDF | |
| | KEDGNILGHKLEYNYNSHNVYIMADKQKNGIKVNFKIRHNIED | |
| | GSVQLADHYQQNTPIGDGPVLLPDNHYLSTQSALSKDPNEKRD | |
| | HMVLLEFVTAAGITLGMDELYK | |
| TOB2 | MQLEIKVALNFIISYLYNKLPRRRADLFGEELERLLKKKYEGHW | 114 |
| | YPEKPLKGSGFRCVHIGEMVDPVVELAAKRSGLAVEDVRANVP | |
| | EELSVWIDPFEVSYQIGEKGAVKVLYLDDSEGCGAPELDKEIKS | |
| | SFNPDAQVFVPIGSQDSSLSNSPSPSFGQSPSPTFIPRSAQPITFTT | |
| | ASFAATKFGSTKMKKGGGAASGGGVASSGAGGQQPPQQPRM | |
| | ARSPTNSLLKHKSLSLSMHSLNFITANPAPQSQLSPNAKEFVYN | |
| | GGGSPSLFFDAADGQGSGTPGPFGGSGAGTCNSSSFDMAQVFG | |
| | GGANSLFLEKTPFVEGLSYNLNTMQYPSQQFQPVVLAN | |
| RNase4 | MQDGMYQRFLRQHVHPEETGGSDRYCNLMMQRRKMTLYHC | 115 |
| | KRFNTFIHEDIWNIRSICSTTNIQCKNGKMNCHEGVVKVTDCRD | |
| | TGSSRAPNCRYRAIASTRRVVIACEGNPQVPVHFDG | |
| RNaseL | WKPQSSHWGAALKDLHRIYRPMIGKLKFFIDEKYKIADTSEGGI | 116 |
| | YLGFYEKQEVAVKTFCEGSPRAQREVSCLQSSRENSHLVTFYG | |
| | SESHRGHLFVCVTLCEQTLEACLDVHRGEDVENEEDEFARNVL | |
| | SSIFKAVQELHLSCGYTHQDLQPQNILIDSKKAAHLADFDKSIK | |
| | WAGDPQEVKRDLEDLGRLVLYVVKKGSISFEDLKAQSNEEVV | |
| | QLSPDEETKDLIHRLFHPGEHVRDCLSDLLGHPFFWTWESRYRT | |
| | LRNVGNESDIKTRKSESEILRLLQPGPSEHSKSFDKWTTKINECV | |
| | MKKMNKFYEKRGNFYQNTVGDLLKFIRNLGEHIDEEKHKKMK | |
| | LKIGDPSLYFQKTFPDLVIYVYTKLQNTEYRKHFPQTHSPNKPQ | |
| | CDGAGGASGLASPGC | |
| RNaseA | ETSAEKFERQHMDADSSPASNSNYCNKMMKSRSMTTDRCKPV | 117 |
| | NTFIHEPLADVQAVCSQTNVACKNGQTNCYQSKAAMRITDCRE | |
| | TGSSKYPDCAYTTTEEEKHIIVACEGNPYVPVHFDATV | |
| RNase1 | MKESRAKKFQRQHMDSDSSPSSSSTYCNQMMRRRNMTQGRC | 118 |
| | KPVNTFVHEPLVDVQNVCFQEKVTCKNGQGNCYKSNSSMHIT | |
| | DCRLINGSRYPNCAYRTSPKERHIIVACEGSPYVPVHFDASVED | |
| | ST | |
| RNaseU2 | CDIPQSTNCGGNVYSNDDINTAIQGALDDVANGDRPDNYPHQY | 119 |
| | YDEASEDITLCCGSGPWSEFPLVYNGPYYSSRDNYVSPGPDRVI | |
| | YQTNTGEFCATVTHTGAASYDGFTQCS | |
| HRSP12 | MSSLIRRVISTAKAPGAIGPYSQAVLVDRTIYISGQIGMDPSSGQ | 120 |
| | LVSGGVAEEAKQALKNMGEILKAAGCDFTNVVKTTVLLADIN | |
| | DFNTVNEIYKQYFKSNFPARAAYQVAALPKGSRIEIEAVAIQGP | |
| | LTTASL | |
| RNaseP | MENFRKVRSEEAPAGCGAEGGGPGSGPFADLAPGAVHMRVKE | 121 |
| (RPP25) | GSKIRNLMAFATASMAQPATRAIVFSGCGRATTKTVTCAEILKR | |
| | RLAGLHQVTRLRYRSVREVWQSLPPGPTQGQTPGEPAASLSVL | |
| | KNVPGLAILLSKDALDPRQPGYQPPNPHPGPSSPPAAPASKRSL | |
| | GEPAAGEGSAKRSQPEPGVADEDQTA | |

TABLE
Full CIRTS sequences used in FIG. 4: (Bdef-(GGS)₆-TBP6.7-NES-(GGS)₆-Effector-
Tag

| | | SEQ ID |
|---|---|---|
| Description | Sequence | NO: |
| GFP | MGIINTLQKYYCRVRGGRCAVLSCLPKEEQIGKCSTRGRKC | 148 |
| | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSVSKGEELFT | |
| | GVVPILVELDGDVNGHKFSVSGEGEGDATYGKLTLKFICTT | |
| | GKLPVPWPTLVTTLTYGVQCFSRYPDHMKQHDFFKSAMPE | |
| | GYVQERTIFFKDDGNYKTRAEVKFEGDTLVNRIELKGIDFKE | |
| | DGNILGHKLEYNYNSHNVYIMADKQKNGIKVNFKIRHNIED | |
| | GSVQLADHYQQNTPIGDGPVLLPDNHYLSTQSALSKDPNEK | |
| | RDHMVLLEFVTAAGITLGMDELYKGSDYKDDDDK | |
| Y2 (1-191) | MGIINTLQKYYCRVRGGRCAVLSCLPKEEQIGKCSTRGRKC | 149 |
| | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSMSASSLLEQ | |
| | RPKGQGNKVQNGSVHQKDGLNDDDFEPYLSPQARPNNAYT | |
| | AMSDSYLPSYYSPSIGFSYSLGEAAWSTGGDTAMPYLTSYG | |
| | QLSNGEPHFLPDAMFGQPGALGSTPFLGQHGFNFFPSGIDFS | |
| | AWGNNSSQGQSTQSSGYSSNYAYAPSSLGGAMIDGQSAFA | |
| | NETLNKAPGMNTIDQGMAAGSDYKDDDDK | |
| CNOT7 | MGIINTLQKYYCRVRGGRCAVLSCLPKEEQIGKCSTRGRKC | 150 |
| | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSMPAATVDHS | |
| | QRICEVWACNLDEEMKKIRQVIRKYNYVAMDTEFPGVVAR | |
| | PIGEFRSNADYQYQLLRCNVDLLKIIQLGLTFMNEQGEYPPG | |
| | TSTWQFNFKFNLTEDMYAQDSIELLTTSGIQFKKHEEEGIET | |
| | QYFAELLMTSGVVLCEGVKWLSFHSGYDFGYLIKILTNSNL | |
| | PEEELDFFEILRLFFPVIYDVKYLMKSCKNLKGGLQEVAEQL | |
| | ELERIGPQHQAGSDSLLTGMAFFKMREMFFEDHIDDAKYCG | |
| | HLYGLGSGSSYVQNGTGNAYEEEANKQSGSDYKDDDDK | |
| SNRPA | MGIINTLQKYYCRVRGGRCAVLSCLPKEEQIGKCSTRGRKC | 63 |
| | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSMAVPETRPN | |
| | HTIYINNLNEKIKKDELKKSLYAIFSQFGQILDILVSRSLKMR | |
| | GQAFVIFKEVSSATNALRSMQGFPFYDKPMRIQYAKTDSDII | |
| | AKMKGTFVERDRKREKRKPKSQETPATKKAVQGGGATPVV | |
| | GAVQGPVPGMPPMTQAPRIMHHMPGQPPYMPPPGMIPPPG | |
| | LAPGQIPPGAMPPQQLMPGQMPPAQPLSENPPNHILFLTNLP | |
| | EETNELMLSMLFNQFPGFKEVRLVPGRHDIAFVEFDNEVQA | |
| | GAARDALQGFKITQNNAMKISFAKKGSDYKDDDDK | |
| ZFP36 | MGIINTLQKYYCRVRGGRCAVLSCLPKEEQIGKCSTRGRKC | 151 |
| | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSMDLTAIYESL | |
| | LSLSPDVPVPSDHGGTESSPGWGSSGPWSLSPSDSSPSGVTS | |
| | RLPGRSTSLVEGRSCGWVPPPPGFAPLAPRLGPELSPSPTSPT | |
| | ATSTTPSRYKTELCRTFSESGRCRYGAKCQFAHGLGELRQA | |
| | NRHPKYKTELCHKFYLQGRCPYGSRCHFIHNPSEDLAAPGH | |
| | PPVLRQSISFSGLPSGRRTSPPPPGLAGPSLSSSSFSPSSSPPPP | |
| | GDLPLSPSAFSAAPGTPLARRDPTPVCCPSCRRATPISVWGP | |
| | LGGLVRTPSVQSLGSDPDEYASSGSSLGGSDSPVFEAGVFAP | |
| | PQPVAAPRRLPIFNRISVSEGSDYKDDDDK | |
| TOB2 | MGIINTLQKYYCRVRGGRCAVLSCLPKEEQIGKCSTRGRKC | 152 |
| | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSMQLEIKVAL | |
| | NFIISYLYNKLPRRRADLFGEELERLLKKKYEGHWYPEKPLK | |
| | GSGFRCVHIGEMVDPVVELAAKRSGLAVEDVRANVPEELS | |
| | VWIDPFEVSYQIGEKGAVKVLYLDDSEGCGAPELDKEIKSSF | |
| | NPDAQVFVPIGSQDSSLSNSPSPSFGQSPSPTFIPRSAQPITFTT | |
| | ASFAATKFGSTKMKKGGGAASGGGVASSGAGGQQPPQQPR | |
| | MARSPTNSLLKHKSLSLSMHSLNFITANPAPQSQLSPNAKEF | |
| | VYNGGGSPSLFFDAADGQGSGTPGPFGGSGAGTCNSSSFDM | |
| | AQVFGGGANSLFLEKTPFVEGLSYNLNTMQYPSQQFQPVVL | |
| | ANGSDYKDDDDK | |
| RNase4 | MGIINTLQKYYCRVRGGRCAVLSCLPKEEQIGKCSTRGRKC | 153 |
| | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSMQDGMYQR | |
| | FLRQHVHPEETGGSDRYCNLMMQRRKMTLYHCKRFNTFIH | |
| | EDIWNIRSICSTTNIQCKNGKMNCHEGVVKVTDCRDTGSSR | |
| | APNCRYRAIASTRRVVIACEGNPQVPVHFDGGSDYKDDDD | |
| | K | |
| RNaseL | MGIINTLQKYYCRVRGGRCAVLSCLPKEEQIGKCSTRGRKC | 154 |
| | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSWKPQSSHW | |
| | GAALKDLHRIYRPMIGKLKFFIDEKYKIADTSEGGIYLGFYE | |
| | KQEVAVKTFCEGSPRAQREVSCLQSSRENSHLVTFYGSESH | |
| | RGHLFVCVTLCEQTLEACLDVHRGEDVENEEDEFARNVLSS | |
| | IFKAVQELHLSCGYTHQDLQPQNILIDSKKAAHLADFDKSIK | |
| | WAGDPQEVKRDLEDLGRLVLYVVKKGSISFEDLKAQSNEE | |
| | VVQLSPDEETKDLIHRLFHPGEHVRDCLSDLLGHPFFWTWE | |
| | SRYRTLRNVGNESDIKTRKSESEILRLLQPGPSEHSKSFDKW | |
| | TTKINECVMKKMNKFYEKRGNFYQNTVGDLLKFIRNLGEHI | |
| | DEEKHKKMKLKIGDPSLYFQKTFPDLVIYVYTKLQNTEYRK | |
| | HFPQTHSPNKPQCDGAGGASGLASPGCGSDYKDDDDK | |
| RNaseA | MGIINTLQKYYCRVRGGRCAVLSCLPKEEQIGKCSTRGRKC | 155 |
| | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSETSAEKFERQ | |
| | HMDADSSPASNSNYCNKMMKSRSMTTDRCKPVNTFIHEPL | |
| | ADVQAVCSQTNVACKNGQTNCYQSKAAMRITDCRETGSSK | |
| | YPDCAYTTTEEEKHIIVACEGNPYVPVHFDATVGSDYKDDD | |
| | DK | |
| Pin nuclease | MGIINTLQKYYCRVRGGRCAVLSCLPKEEQIGKCSTRGRKC | 156 |
| | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSMELEIRPLFL | |
| | VPDTNGFIDHLASLARLLESRKYILVVPLIVINELDGLAKGQE | |
| | TDHRAGGYARVVQEKARKSIEFLEQRFESRDSCLRALTSRG | |
| | NELESIAFRSEDITGQLGNNDDLILSCCLHYCKDKAKDFMPA | |
| | SKEEPIRLLREVVLLTDDRNLRVKALTRNVPVRDIPAFLTWA | |
| | QVGGSDYKDDDDK | |
| RNase1 | MGIINTLQKYYCRVRGGRCAVLSCLPKEEQIGKCSTRGRKC | 157 |
| | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSMKESRAKKF | |
| | QRQHMDSDSSPSSSSTYCNQMMRRRNMTQGRCKPVNTFVH | |
| | EPLVDVQNVCFQEKVTCKNGQGNCYKSNSSMHITDCRLTN | |
| | GSRYPNCAYRTSPKERHIIVACEGSPYVPVHFDASVEDSTGS | |
| | DYKDDDDK | |
| RNaseU2 | MGIINTLQKYYCRVRGGRCAVLSCLPKEEQIGKCSTRGRKC | 158 |
| | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSCDIPQSTNCG | |
| | GNVYSNDDINTAIQGALDDVANGDRPDNYPHQYYDEASED | |
| | ITLCCGSGPWSEFPLVYNGPYYSSRDNYVSPGPDRVIYQTNT | |
| | GEFCATVTHTGAASYDGFTQCSGSDYKDDDDK | |
| HRSP12 | MGIINTLQKYYCRVRGGRCAVLSCLPKEEQIGKCSTRGRKC | 159 |
| | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSMSSLIRRVIS | |
| | TAKAPGAIGPYSQAVLVDRTIYISGQIGMDPSSGQLVSGGVA | |
| | EEAKQALKNMGEILKAAGCDFTNVVKTTVLLADINDFNTV | |
| | NEIYKQYFKSNFPARAAYQVAALPKGSRIEIEAVAIQGPLTT | |
| | ASLGSDYKDDDDK | |
| RNaseP | MGIINTLQKYYCRVRGGRCAVLSCLPKEEQIGKCSTRGRKC | 160 |
| (RPP25) | CRRKKGGSGGSGGSGGSGGSGGSMAVPETRPNHTIYINNLN | |
| | SKIKKDELKKSLYAIFSQFGQILDILVPRQRTPRGQAFVIFKE | |
| | VSSATNALRSMQGFPFYDKPMRIQYAKTDKRIPAKMKGTF | |
| | VGSLQLPPLERLTLGGSGGSGGSGGSGGSGGSMENFRKVRS | |
| | EEAPAGCGAEGGGPGSGPFADLAPGAVHMRVKEGSKIRNL | |
| | MAFATASMAQPATRAIVESGCGRATTKTVTCAEILKRRLAG | |
| | LHQVTRLRYRSVREVWQSLPPGPTQGQTPGEPAASLSVLKN | |
| | VPGLAILLSKDALDPRQPGYQPPNPHPGPSSPPAAPASKRSL | |
| | GEPAAGEGSAKRSQPEPGVADEDQTAGSDYKDDDDK | |

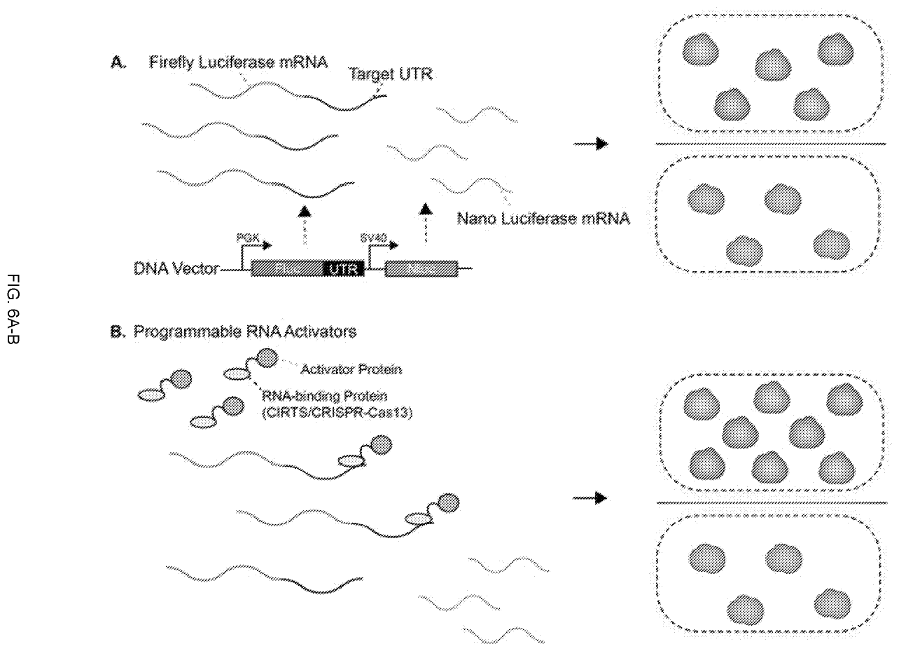

### B. FIG. 5 Methods and Results

Methods: HEK293T cells were plated on 96-well plates and transfected at 60% confluency 24 hours later. 250 ng of the indicated gRNA-CIRTS expression plasmids was transfected per well using Lipofectamine 2000 and Opti-MEM I Reduced Serum Medium (ThermoFisher Scientific) according to the manufacture's protocol. After 48 hours, total RNA was harvested and isolated using the RNeasy Mini Kit (QIAGEN). After RNA isolation, RNA was reverse transcribed to cDNA using the PrimeScript RT Reagent Kit (TaKaRa). All qPCR reactions were run at 20 AL volumes with at least three biological replicates using PowerUp SYBR Green Master Mix (Applied Biosystems) and amplified on a Quantstudio 6 (Applied Biosystems). All qPCR primers used were designed on NCBI Primer BLAST and validated by no measured amplification in the absence of cDNA, a sharp, single-product melting temperature, and an amplification efficiency within 95-105%.

Expression levels were calculated using the housekeeping control gene (beta-actin) cycle threshold (Ct) value and the gene of interest Ct value. The relative expression level of SNCA was determined by 2dCt, where dCt=Ct(SNCA)−Ct(beta-actin). Relative expression level for targeted gene was obtained upon normalizing the targeted gene expression level of cells experiments treated with the on-target gRNA to those treated by the nontargeting (NT) gRNA.

Results: We deployed CIRTS-Y2 with a gRNA panel targeting SNCA in HEK293T cells and demonstrated that CIRTS-Y2 can degrade SNCA transcript levels in a gRNA-dependent manner.

gRNA sequences used in FIG. 5

| | | SEQ ID |
|---|---|---|
| Description | Sequence | NO: |
| g1 | TAATGAATTCCTTTACACCACACTGTCGTCGAATGGCCAC | 122 |
| g2 | TCTTCTCAGCCACTGTTGCCACACCATGCACCACTCCCTC | 123 |
| g3 | AGAAGGCATTTCATAAGCCTCATTGTCAGGATCCACAGGC | 124 |
| g4 | TTAACATCGTAGATTGAAGCCACAAAATCCACAGCACACA | 125 |
| g5 | TTTCAATAATTAATCACAGCCACTTAAGGAACCAGTGCAT | 126 |
| g6 | CGAATATTATTTATTGTCAGAAAGGTACAGCATTCACACC | 127 |
| g7 | GCAACATTTAAAGGAGGCCATGAAATTTTTATCAGTCTAG | 128 |
| g8 | CAGAGTTTTTCACCATCAAGTTTATTTTCAGCACCCATGG | 129 |
| g9 | CTACTAGTGACTTTTAGAAATGACTATGCCCCAGAATAAT | 130 |
| g10 | TAGATTTAAAATATATCCTAACCGCCACTTTCTAACCTTC | 131 |
| | | SEQ ID |
| Sequences of qPCR primers used in FIG. 5 | | NO: |
| SNCA Primer 1 | CAACAGTGGCTGAGAAGACCA | 132 |
| SNCA Primer 2 | GCTCCTTCTTCATTCTTGCCCA | 133 |
| Beta-actin | CACCAACTGGGACGACAT | 134 |
| Primer 1 | | |
| Beta-actin | ACAGCCTGGATAGCAACG | 135 |
| Primer 2 | | |

### Example 5: CIRTS Activators

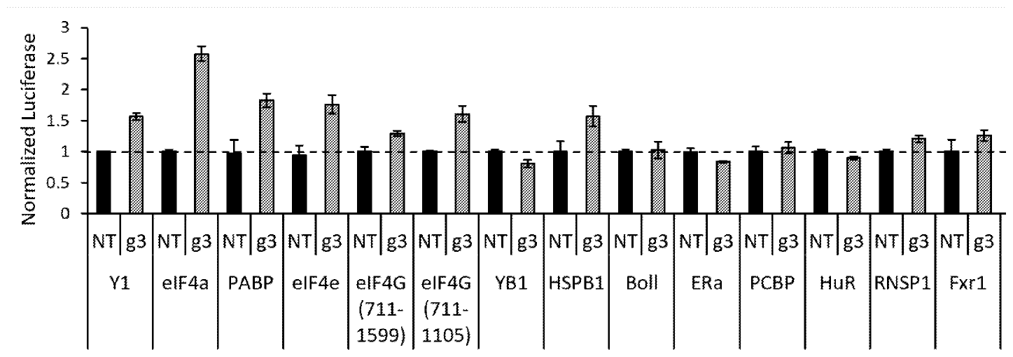

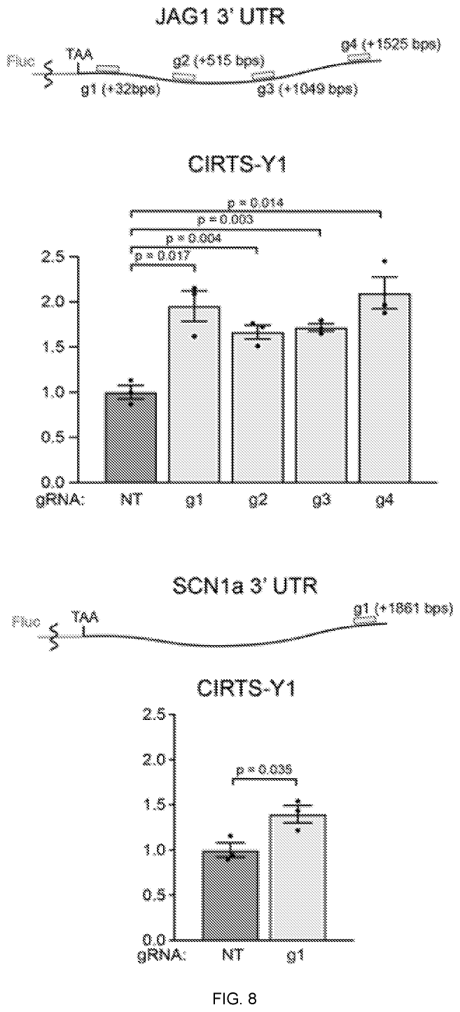

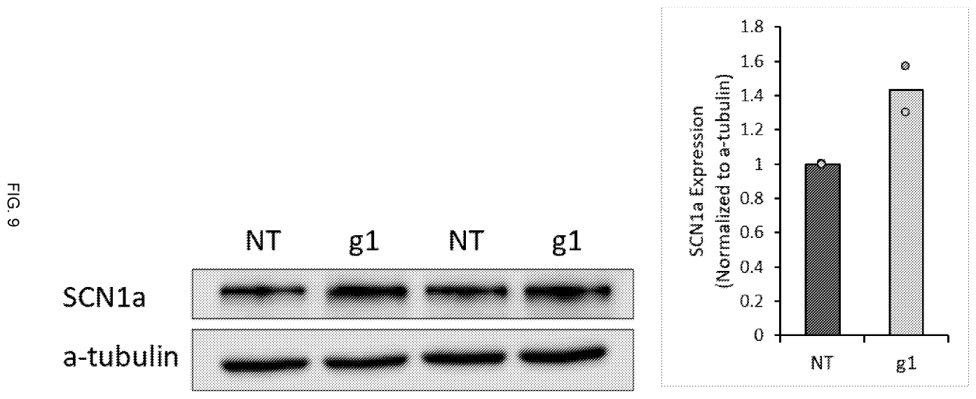

FIG. 6 graphically represents the dual luciferase assays carried out in FIG. 7 and FIG. 8 to characterize the ability of novel CIRTS activators to increase Firefly luciferase levels in a gRNA-dependent manner.

### A. FIG. 7 Methods and Results

Methods: Gibson Assembly was used to assemble all plasmids and plasmids were sequenced by the University of Chicago Comprehensive Cancer Center DNA Sequencing and Genotyping Facility. PCR fragments used for Gibson Assembly were amplified with Q5 DNA Polymerase (NEB). All transcript UTR regions were either cloned from gBlock (IDT) or amplified from reverse transcription reactions completed with total RNA isolated from Neuro 2a (ATCC) cells for SCN1a 3′ UTR.

HEK293T cells were plated on 96-well plates and transfected at 70% confluency 24 hours later. 250 ng of the indicated gRNA-CIRTS expression plasmids and 12 ng reporter plasmid were transfected per well using Lipofectamine 2000 and Opti-MEM I Reduced Serum Medium (ThermoFisher Scientific) according to the manufacture's protocol. After 48 hours, luciferase activity for both Firefly and nano luciferase was assayed according to protocols previously developed and described by Baker et. al (using the nano luciferase in place of Renilla luciferase). Firefly luciferase values were then normalized to the corresponding nano luciferase readout by dividing the firefly RLU by nano luciferase RLU for each individual well. Fold-changes were then calculated by averaging the luciferase ratio for the negative control and dividing all biological replicates by the negative control's luciferase ratio (see, for example, J. M. Baker and F. M. Boyce, J. Vis. Exp. JoVE, 2014, 50282).

Results: Plasmids were constructed in order to develop a dual-luciferase reporter assay which expresses a luciferase transcript appended with the mouse PMP22 transcript 5′ and 3′ UTR regions. CIRTS targeting the PMP22 UTRs that affect translation create changes in the luminescence readout that can be readily normalized by a second, untargeted luciferase (nano luciferase) also expressed from the reporter construct. This reporter system was leveraged to test previously reported (Y1) and novel CIRTS activators for the ability to increase luciferase expression when directed to the reporter's PMP22 3′ UTR (g3) compared to effector-matched constructs with non-targeting (NT) gRNA. These results demonstrate several novel CIRTS with the ability to increase firefly expression in a gRNA-dependent manner.

TABLE
Effectors

| | | SEQ |
|---|---|---|
| | | ID |
| Description | Sequence | NO:: |
| PABP | MNPSAPSYPMASLYVGDLHPDVTEAMLYEKFSPAGPILSIRVCR | 136 |
| | DMITRRSLGYAYVNFQQPADAERALDTMNFDVIKGKPVRIMWS | |
| | QRDPSLRKSGVGNIFIKNLDKSIDNKALYDTFSAFGNILSCKVVC | |
| | DENGSKGYGFVHFETQEAAERAIEKMNGMLLNDRKVFVGRFKS | |
| | RKEREAELGARAKEFTNVYIKNFGEDMDDERLKDLFGKFGPAL | |
| | SVKVMTDESGKSKGFGFVSFERHEDAQKAVDEMNGKELNGKQI | |
| | YVGRAQKKVERQTELKRKFEQMKQDRITRYQGVNLYVKNLDD | |
| | GIDDERLRKEFSPFGTITSAKVMMEGGRSKGFGFVCFSSPEEATK | |
| | AVTEMNGRIVATKPLYVALAQRKEERQAHLTNQYMQRMASVR | |
| | AVPNPVINPYQPAPPSGYFMAAIPQTQNRAAYYPPSQIAQLRPSP | |
| | RWTAQGARPHPFQNMPGAIRPAAPRPPFSTMRPASSQVPRVMST | |
| | QRVANTSTQTMGPRPAAAAAAATPAVRTVPQYKYAAGVRNPQ | |
| | QHLNAQPQVTMQQPAVHVQGQEPLTASMLASAPPQEQKQMLG | |
| | ERLFPLIQAMHPTLAGKITGMLLEIDNSELLHMLESPESLRSKVD | |
| | EAVAVLQAHQAKEAAQKAVNSATGVPTV | |
| eIF4G (711- | EPRKIIATVLMTEDIKLNKAEKAWKPSSKRTAADKDRGEEDADG | 137 |
| 1599) | SKTQDLFRRVRSILNKLTPQMFQQLMKQVTQLAIDTEERLKGVI | |
| | DLIFEKAISEPNFSVAYANMCRCLMALKVPTTEKPTVTVNFRKL | |
| | LLNRCQKEFEKDKDDDEVFEKKQKEMDEAATAEERGRLKEELE | |
| | EARDIARRRSLGNIKFIGELFKLKMLTEAIMHDCVVKLLKNHDE | |
| | ESLECLCRLLTTIGKDLDFEKAKPRMDQYFNQMEKIIKEKKTSSR | |
| | IRFMLQDVLDLRGSNWVPRRGDQGPKTIDQIHKEAEMEEHREHI | |
| | KVQQLMAKGSDKRRGGPPGPPISRGLPLVDDGGWNTVPISKGSR | |
| | PIDTSRLTKITKPGSIDSNNQLFAPGGRLSWGKGSSGGSGAKPSD | |
| | AASEAARPATSTLNRFSALQQAVPTESTDNRRVVQRSSLSRERG | |
| | EKAGDRGDRLERSERGGDRGDRLDRARTPATKRSFSKEVEERSR | |
| | ERPSQPEGLRKAASLTEDRDRGRDAVKREAALPPVSPLKAALSE | |
| | EELEKKSKAIIEEYLHLNDMKEAVQCVQELASPSLLFIFVRHGVE | |
| | STLERSAIAREHMGQLLHQLLCAGHLSTAQYYQGLYEILELAED | |
| | MEIDIPHVWLYLAELVTPILQEGGVPMGELFREITKPLRPLGKAA | |
| | SLLLEILGLLCKSMGPKKVGTLWREAGLSWKEFLPEGQDIGAFV | |
| | AEQKVEYTLGEESEAPGQRALPSEELNRQLEKLLKEGSSNQRVF | |
| | DWIEANLSEQQIVSNTLVRALMTAVCYSAIIFETPLRVDVAVLK | |
| | ARAKLLQKYLCDEQKELQALYALQALVVTLEQPPNLLRMFFDA | |
| | LYDEDVVKEDAFYSWESSKDPAEQQGKGVALKSVTAFFKWLR | |
| | EAEEESDHN | |
| eIF4G (711- | EPRKIIATVLMTEDIKLNKAEKAWKPSSKRTAADKDRGEEDADG | 138 |
| 1105) | SKTQDLFRRVRSILNKLTPQMFQQLMKQVTQLAIDTEERLKGVI | |
| | DLIFEKAISEPNFSVAYANMCRCLMALKVPTTEKPTVTVNFRKL | |
| | LLNRCQKEFEKDKDDDEVFEKKQKEMDEAATAEERGRLKEELE | |
| | EARDIARRRSLGNIKFIGELFKLKMLTEAIMHDCVVKLLKNHDE | |
| | ESLECLCRLLTTIGKDLDFEKAKPRMDQYFNQMEKIIKEKKTSSR | |
| | IRFMLQDVLDLRGSNWVPRRGDQGPKTIDQIHKEAEMEEHREHI | |
| | KVQQLMAKGSDKRRGGPPGPPISRGLPLVDDGGWNTVPISKGSR | |
| | PIDTSRLTKITKPGSIDSNNQLFAPGGRLSWGKGSSGGSGAK | |
| Boll | MQTDSLSPSPNPVSPVPLNNPTSAPRYGTVIPNRIFVGGIDFKTNE | 139 |
| | SDLRKFFSQYGSVKEVKIVNDRAGVSKGYGFVTFETQEDAQKIL | |
| | QEAEKLNYKDKKLNIGPAIRKQQVGIPRSSIMPAAGTMYLTTST | |
| | GYPYTYHNGVAYFHTPEVTSVPPPWPSRSVCSSPVMVAQPIYQQ | |
| | PAYHYQATTQYLPGQWQWSVPQPSASSAPFLYLQPSEVIYQPVE | |
| | IAQDGGCVPPPLSLMETSVPEPYSDHGVQATYHQVYAPSAITMP | |
| | APVMQPEPIKTVWSIHY | |
| ERa | MTMTLHTKASGMALLHQIQGNELEPLNRPQLKIPLERPLGEVYL | 140 |
| | DSSKPAVYNYPEGAAYEFNAAAAANAQVYGQTGLPYGPGSEA | |
| | AAFGSNGLGGFPPLNSVSPSPLMLLHPPPQLSPFLQPHGQQVPYY | |
| | LENEPSGYTVREAGPPAFYRPNSDNRRQGGRERLASTNDKGSM | |
| | AMESAKETRYCAVCNDYASGYHYGVWSCEGCKAFFKRSIQGH | |
| | NDYMCPATNQCTIDKNRRKSCQACRLRKCYEVGMMKGGIRKD | |
| | RRGGRMLKHKRQRDDGEGRGEVGSAGDMRAANLWPSPLMIKR | |
| | SKKNSLALSLTADQMVSALLDAEPPILYSEYDPTRPFSEASMMG | |
| | LLTNLADRELVHMINWAKRVPGFVDLTLHDQVHLLECAWLEIL | |
| | MIGLVWRSMEHPGKLLFAPNLLLDRNQGKCVEGMVEIFDMLLA | |
| | TSSRFRMMNLQGEEFVCLKSIILLNSGVYTFLSSTLKSLEEKDHIH | |
| | RVLDKITDTLIHLMAKAGLTLQQQHQRLAQLLLILSHIRHMSNK | |
| | GMEHLYSMKCKNVVPLYDLLLEMLDAHRLHAPTSRGGASVEE | |
| | TDQSHLATAGSTSSHSLQKYYITGEAEGFPATV | |
| PCBP | MDTGVIEGGLNVTLTIRLLMHGKEVGSIIGKKGESVKKMREESG | 141 |
| | ARINISEGNCPERIITLAGPTNAIFKAFAMIIDKLEEDISSSMTNST | |
| | AASRPPVTLRLVVPASQCGSLIGKGGCKIKEIRESTGAQVQVAG | |
| | DMLPNSTERAITIAGIPQSIIECVKQICVVMLESPPKGVTIPYRPKP | |
| | SSSPVIFAGGQAYTIQGQYAIPQPDLTKLHQLAMQQSHFPMTHG | |
| | NTGFSGIESSSPEVKGYWAGLDASAQTTSHELTIPNDLIGCIIGRQ | |
| | GAKINEIRQMSGAQIKIANPVEGSTDRQVTITGSAASISLAQYLIN | |
| | VRLSSETGGMGSS | |
| HuR | MSNGYEDHMAEDCRGDIGRTNLIVNYLPQNMTQDELRSLESSIG | 142 |
| | EVESAKLIRDKVAGHSLGYGFVNYVTAKDAERAINTLNGLRLQS | |
| | KTIKVSYARPSSEVIKDANLYISGLPRTMTQKDVEDMFSRFGRII | |
| | NSRVLVDQTTGLSRGVAFIRFDKRSEAEEAITSFNGHKPPGSSEPI | |
| | TVKFAANPNQNKNVALLSQLYHSPARRFGGPVHHQAQRFRFSP | |
| | MGVDHMSGLSGVNVPGNASSGWCIFIYNLGQDADEGILWQMF | |
| | GPFGAVTNVKVIRDFNTNKCKGFGFVTMTNYEEAAMAIASLNG | |
| | YRLGDKILQVSFKTNKSHK | |
| RNSP1 | MDLSGVKKKSLLGVKENNKKSSTRAPSPTKRKDRSDEKSKDRS | 143 |
| | KDKGATKESSEKDRGRDKTRKRRSASSGSSSTRSRSSSTSSSGSS | |
| | TSTGSSSGSSSSSASSRSGSSSTSRSSSSSSSSGSPSPSRRRHDNRRR | |
| | SRSKSKPPKRDEKERKRRSPSPKPTKVHIGRLTRNVTKDHIMEIFS | |
| | TYGKIKMIDMPVERMHPHLSKGYAYVEFENPDEAEKALKHMD | |
| | GGQIDGQEITATAVLAPWPRPPPRRFSPPRRMLPPPPMWRRSPPR | |
| | MRRRSRSPRRRSPVRRRSRSPGRRRHRSRSSSNSSR | |

TABLE
Full CIRTS sequences used in FIG. 7: (Bdef-(GGS)_{₆}-TBP6.7-NES-(GGS)_{₆}-Effector-
Tag

| | | SEQ |
|---|---|---|
| | | ID |
| Description | Sequence | NO: |
| Y1 (1-364) | MGIINTLQKYYCRVRGGRCAVLSCLPKEEQIGKCSTRGRKCCR | 80 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMSATSVDTQRTKGQDNKVQ | |
| | NGSLHQKDTVHDNDFEPYLTGQSNQSNSYPSMSDPYLSSYYPP | |
| | SIGFPYSLNEAPWSTAGDPPIPYLTTYGQLSNGDHHFMHDAVFG | |
| | QPGGLGNNIYQHRFNFFPENPAFSAWGTSGSQGQQTQSSAYGS | |
| | SYTYPPSSLGGTVVDGQPGFHSDTLSKAPGMNSLEQGMVGLKI | |
| | GDVSSSAVKTVGSVVSSVALTGVLSGNGGTNVNMPVSKPTSW | |
| | AAIASKPAKPQPKMKTKSGPVMGGGLPPPPIKHNMDIGTWDNK | |
| | GPVPKAPVPQQAPSPQAAPQPQQVAQPLPAQPPALAQPQYQSP | |
| | QQPPQTRWVAPRNRNAAFGQSGGAGSDSNSPGNVQPNSAPSV | |
| | ESGSDYKDDDDK | |
| eIF4a | MGIINTLQKYYCRVRGGRCAVLSCLPKEEQIGKCSTRGRKCCR | 161 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMSASQDSRSRDNGPDGMEPE | |
| | GVIESNWNEIVDSFDDMNLSESLLRGIYAYGFEKPSAIQQRAILP | |
| | CIKGYDVIAQAQSGTGKTATFAISILQQIELDLKATQALVLAPTR | |
| | ELAQQIQKVVMALGDYMGASCHACIGGTNVRAEVQKLQMEA | |
| | PHIIVGTPGRVFDMLNRRYLSPKYIKMFVLDEADEMLSRGFKD | |
| | QIYDIFQKLNSNTQVVLLSATMPSDVLEVTKKFMRDPIRILVKK | |
| | EELTLEGIRQFYINVEREEWKLDTLCDLYETLTITQAVIFINTRR | |
| | KVDWLTEKMHARDFTVSAMHGDMDQKERDVIMREFRSGSSR | |
| | VLITTDLLARGIDVQQVSLVINYDLPTNRENYIHRIGRGGRFGRK | |
| | GVAINMVTEEDKRTLRDIETFYNTSIEEMPLNVADLIGSDYKDD | |
| | DDK | |
| eIF4e | MGIINTLQKYYCRVRGGRCAVLSCLPKEEQIGKCSTRGRKCCR | 81 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMATVEPETTPTPNPPTTEEEK | |
| | TESNQEVANPEHYIKHPLQNRWALWFFKNDKSKTWQANLRLIS | |
| | KFDTVEDFWALYNHIQLSSNLMPGCDYSLFKDGIEPMWEDEKN | |
| | KRGGRWLITLNKQQRRSDLDRFWLETLLCLIGESFDDYSDDVC | |
| | GAVVNVRAKGDKIAIWTTECENREAVTHIGRVYKERLGLPPKI | |
| | VIGYQSHADTATKSGSTTKNRFVVGSDYKDDDDK | |
| YB1 | MGIINTLQKYYCRVRGGRCAVLSCLPKEEQIGKCSTRGRKCCR | 66 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMSSEAETQQPPAAPPAAPALS | |
| | AADTKPGTTGSGAGSGGPGGLTSAAPAGGDKKVIATKVLGTV | |
| | KWFNVRNGYGFINRNDTKEDVFVHQTAIKKNNPRKYLRSVGD | |
| | GETVEFDVVEGEKGAEAANVTGPGGVPVQGSKYAADRNHYRR | |
| | YPRRRGPPRNYQQNYQNSESGEKNEGSESAPEGQAQQRRPYRR | |
| | RRFPPYYMRRPYGRRPQYSNPPVQGEVMEGADNQGAGEQGRP | |
| | VRQNMYRGYRPRFRRGPPRQRQPREDGNEEDKENQGDETQGQ | |
| | QPPQRRYRRNFNYRRRRPENPKPQDGKETKAADPPAENSSAPE | |
| | AEQGGAEGSDYKDDDDK | |
| HSPB1 | MGIINTLQKYYCRVRGGRCAVLSCLPKEEQIGKCSTRGRKCCR | 11 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMTERRVPFSLLRGPSWDPFR | |
| | DWYPHSRLFDQAFGLPRLPEEWSQWLGGSSWPGYVRPLPPAAI | |
| | ESPAVAAPAYSRALSRQLSSGVSEIRHTADRWRVSLDVNHFAP | |
| | DELTVKTKDGVVEITGKHEERQDEHGYISRCFTRKYTLPPGVDP | |
| | TQVSSSLSPEGTLTVEAPMPKLATQSNEITIPVTFESRAQLGGPE | |
| | AAKSDETAAKLGSDYKDDDDK | |
| PABP | MGIINTLQKYYCRVRGGRCAVLSCLPKEEQIGKCSTRGRKCCR | 162 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMNPSAPSYPMASLYVGDLHP | |
| | DVTEAMLYEKFSPAGPILSIRVCRDMITRRSLGYAYVNFQQPAD | |
| | AERALDTMNFDVIKGKPVRIMWSQRDPSLRKSGVGNIFIKNLD | |
| | KSIDNKALYDTFSAFGNILSCKVVCDENGSKGYGFVHFETQEA | |
| | AERAIEKMNGMLLNDRKVFVGRFKSRKEREAELGARAKEFTN | |
| | VYIKNFGEDMDDERLKDLFGKFGPALSVKVMTDESGKSKGFGF | |
| | VSFERHEDAQKAVDEMNGKELNGKQIYVGRAQKKVERQTELK | |
| | RKFEQMKQDRITRYQGVNLYVKNLDDGIDDERLRKEFSPFGTIT | |
| | SAKVMMEGGRSKGFGFVCFSSPEEATKAVTEMNGRIVATKPLY | |
| | VALAQRKEERQAHLTNQYMQRMASVRAVPNPVINPYQPAPPS | |
| | GYFMAAIPQTQNRAAYYPPSQIAQLRPSPRWTAQGARPHPFQN | |
| | MPGAIRPAAPRPPFSTMRPASSQVPRVMSTQRVANTSTQTMGP | |
| | RPAAAAAAATPAVRTVPQYKYAAGVRNPQQHLNAQPQVTMQ | |
| | QPAVHVQGQEPLTASMLASAPPQEQKQMLGERLFPLIQAMHPT | |
| | LAGKITGMLLEIDNSELLHMLESPESLRSKVDEAVAVLQAHQA | |
| | KEAAQKAVNSATGVPTVGSDYKDDDDK | |
| eIF4G (711- | MGIINTLQKYYCRVRGGRCAVLSCLPKEEQIGKCSTRGRKCCR | 163 |
| 1599) | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSEPRKIIATVLMTEDIKLNKAE | |
| | KAWKPSSKRTAADKDRGEEDADGSKTQDLFRRVRSILNKLTPQ | |
| | MFQQLMKQVTQLAIDTEERLKGVIDLIFEKAISEPNFSVAYANM | |
| | CRCLMALKVPTTEKPTVTVNFRKLLLNRCQKEFEKDKDDDEVF | |
| | EKKQKEMDEAATAEERGRLKEELEEARDIARRRSLGNIKFIGEL | |
| | FKLKMLTEAIMHDCVVKLLKNHDEESLECLCRLLTTIGKDLDFE | |
| | KAKPRMDQYFNQMEKIIKEKKTSSRIRFMLQDVLDLRGSNWVP | |
| | RRGDQGPKTIDQIHKEAEMEEHREHIKVQQLMAKGSDKRRGGP | |
| | PGPPISRGLPLVDDGGWNTVPISKGSRPIDTSRLTKITKPGSIDSN | |
| | NQLFAPGGRLSWGKGSSGGSGAKPSDAASEAARPATSTLNRFS | |
| | ALQQAVPTESTDNRRVVQRSSLSRERGEKAGDRGDRLERSERG | |
| | GDRGDRLDRARTPATKRSFSKEVEERSRERPSQPEGLRKAASLT | |
| | EDRDRGRDAVKREAALPPVSPLKAALSEEELEKKSKAIIEEYLH | |
| | LNDMKEAVQCVQELASPSLLFIFVRHGVESTLERSAIAREHMGQ | |
| | LLHQLLCAGHLSTAQYYQGLYEILELAEDMEIDIPHVWLYLAEL | |
| | VTPILQEGGVPMGELFREITKPLRPLGKAASLLLEILGLLCKSMG | |
| | PKKVGTLWREAGLSWKEFLPEGQDIGAFVAEQKVEYTLGEESE | |
| | APGQRALPSEELNRQLEKLLKEGSSNQRVFDWIEANLSEQQIVS | |
| | NTLVRALMTAVCYSAIIFETPLRVDVAVLKARAKLLQKYLCDE | |
| | QKELQALYALQALVVTLEQPPNLLRMFFDALYDEDVVKEDAF | |
| | YSWESSKDPAEQQGKGVALKSVTAFFKWLREAEEESDHNGSD | |
| | YKDDDDK | |
| eIF4G (711- | MGIINTLQKYYCRVRGGRCAVLSCLPKEEQIGKCSTRGRKCCR | 164 |
| 1105) | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSEPRKIIATVLMTEDIKLNKAE | |
| | KAWKPSSKRTAADKDRGEEDADGSKTQDLFRRVRSILNKLTPQ | |
| | MFQQLMKQVTQLAIDTEERLKGVIDLIFEKAISEPNFSVAYANM | |
| | CRCLMALKVPTTEKPTVTVNFRKLLLNRCQKEFEKDKDDDEVF | |
| | EKKQKEMDEAATAEERGRLKEELEEARDIARRRSLGNIKFIGEL | |
| | FKLKMLTEAIMHDCVVKLLKNHDEESLECLCRLLTTIGKDLDFE | |
| | KAKPRMDQYFNQMEKIIKEKKTSSRIRFMLQDVLDLRGSNWVP | |
| | RRGDQGPKTIDQIHKEAEMEEHREHIKVQQLMAKGSDKRRGGP | |
| | PGPPISRGLPLVDDGGWNTVPISKGSRPIDTSRLTKITKPGSIDSN | |
| | NQLFAPGGRLSWGKGSSGGSGAKGSDYKDDDDK | |
| Boll | MGIINTLQKYYCRVRGGRCAVLSCLPKEEQIGKCSTRGRKCCR | 165 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMQTDSLSPSPNPVSPVPLNNP | |
| | TSAPRYGTVIPNRIFVGGIDFKTNESDLRKFFSQYGSVKEVKIVN | |
| | DRAGVSKGYGFVTFETQEDAQKILQEAEKLNYKDKKLNIGPAI | |
| | RKQQVGIPRSSIMPAAGTMYLTTSTGYPYTYHNGVAYFHTPEV | |
| | TSVPPPWPSRSVCSSPVMVAQPIYQQPAYHYQATTQYLPGQWQ | |
| | WSVPQPSASSAPFLYLQPSEVIYQPVEIAQDGGCVPPPLSLMETS | |
| | VPEPYSDHGVQATYHQVYAPSAITMPAPVMQPEPIKTVWSIHY | |
| | GSDYKDDDDK | |
| ERa | MGIINTLQKYYCRVRGGRCAVLSCLPKEEQIGKCSTRGRKCCR | 166 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMTMTLHTKASGMALLHQIQ | |
| | GNELEPLNRPQLKIPLERPLGEVYLDSSKPAVYNYPEGAAYEFN | |
| | AAAAANAQVYGQTGLPYGPGSEAAAFGSNGLGGFPPLNSVSPS | |
| | PLMLLHPPPQLSPFLQPHGQQVPYYLENEPSGYTVREAGPPAFY | |
| | RPNSDNRRQGGRERLASTNDKGSMAMESAKETRYCAVCNDY | |
| | ASGYHYGVWSCEGCKAFFKRSIQGHNDYMCPATNQCTIDKNR | |
| | RKSCQACRLRKCYEVGMMKGGIRKDRRGGRMLKHKRQRDDG | |
| | EGRGEVGSAGDMRAANLWPSPLMIKRSKKNSLALSLTADQMV | |
| | SALLDAEPPILYSEYDPTRPFSEASMMGLLTNLADRELVHMINW | |
| | AKRVPGFVDLTLHDQVHLLECAWLEILMIGLVWRSMEHPGKL | |
| | LFAPNLLLDRNQGKCVEGMVEIFDMLLATSSRFRMMNLQGEEF | |
| | VCLKSIILLNSGVYTFLSSTLKSLEEKDHIHRVLDKITDTLIHLMA | |
| | KAGLTLQQQHQRLAQLLLILSHIRHMSNKGMEHLYSMKCKNV | |
| | VPLYDLLLEMLDAHRLHAPTSRGGASVEETDQSHLATAGSTSS | |
| | HSLQKYYITGEAEGFPATVGSDYKDDDDK | |
| PCBP | MGIINTLQKYYCRVRGGRCAVLSCLPKEEQIGKCSTRGRKCCR | 167 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMDTGVIEGGLNVTLTIRLLM | |
| | HGKEVGSIIGKKGESVKKMREESGARINISEGNCPERIITLAGPT | |
| | NAIFKAFAMIIDKLEEDISSSMTNSTAASRPPVTLRLVVPASQCG | |
| | SLIGKGGCKIKEIRESTGAQVQVAGDMLPNSTERAITIAGIPQSII | |
| | ECVKQICVVMLESPPKGVTIPYRPKPSSSPVIFAGGQAYTIQGQY | |
| | AIPQPDLTKLHQLAMQQSHFPMTHGNTGFSGIESSSPEVKGYW | |
| | AGLDASAQTTSHELTIPNDLIGCIIGRQGAKINEIRQMSGAQIKIA | |
| | NPVEGSTDRQVTITGSAASISLAQYLINVRLSSETGGMGSSGSDY | |
| | KDDDDK | |
| HuR | MGIINTLQKYYCRVRGGRCAVLSCLPKEEQIGKCSTRGRKCCR | 168 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMSNGYEDHMAEDCRGDIGR | |
| | TNLIVNYLPQNMTQDELRSLFSSIGEVESAKLIRDKVAGHSLGY | |
| | GFVNYVTAKDAERAINTLNGLRLQSKTIKVSYARPSSEVIKDAN | |
| | LYISGLPRTMTQKDVEDMFSRFGRIINSRVLVDQTTGLSRGVAFI | |
| | RFDKRSEAEEAITSFNGHKPPGSSEPITVKFAANPNQNKNVALL | |
| | SQLYHSPARRFGGPVHHQAQRFRFSPMGVDHMSGLSGVNVPG | |
| | NASSGWCIFIYNLGQDADEGILWQMFGPFGAVTNVKVIRDENT | |
| | NKCKGFGFVTMTNYEEAAMAIASLNGYRLGDKILQVSFKTNKS | |
| | HKGSDYKDDDDK | |
| RNSP1 | MGIINTLQKYYCRVRGGRCAVLSCLPKEEQIGKCSTRGRKCCR | 169 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMDLSGVKKKSLLGVKENNK | |
| | KSSTRAPSPTKRKDRSDEKSKDRSKDKGATKESSEKDRGRDKT | |
| | RKRRSASSGSSSTRSRSSSTSSSGSSTSTGSSSGSSSSSASSRSGSS | |
| | STSRSSSSSSSSGSPSPSRRRHDNRRRSRSKSKPPKRDEKERKRR | |
| | SPSPKPTKVHIGRLTRNVTKDHIMEIFSTYGKIKMIDMPVERMH | |
| | PHLSKGYAYVEFENPDEAEKALKHMDGGQIDGQEITATAVLAP | |
| | WPRPPPRRFSPPRRMLPPPPMWRRSPPRMRRRSRSPRRRSPVRR | |
| | RSRSPGRRRHRSRSSSNSSRGSDYKDDDDK | |
| FXR1 | MGIINTLQKYYCRVRGGRCAVLSCLPKEEQIGKCSTRGRKCCR | 77 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMAELTVEVRGSNGAFYKGFI | |
| | KDVHEDSLTVVFENNWQPERQVPFNEVRLPPPPDIKKEISEGDE | |
| | VEVYSRANDQEPCGWWLAKVRMMKGEFYVIEYAACDATYNE | |
| | IVTFERLRPVNQNKTVKKNTFFKCTVDVPEDLREACANENAHK | |
| | DFKKAVGACRIFYHPETTQLMILSASEATVKRVNILSDMHLRSI | |
| | RTKLMLMSRNEEATKHLECTKQLAAAFHEEFVVREDLMGLAI | |
| | GTHGSNIQQARKVPGVTAIELDEDTGTFRIYGESADAVKKARGF | |
| | LEFVEDFIQVPRNLVGKVIGKNGKVIQEIVDKSGVVRVRIEGDN | |
| | ENKLPREDGMVPFVFVGTKESIGNVQVLLEYHIAYLKEVEQLR | |
| | MERLQIDEQLRQIGSRSYSGRGRGRRGPNYTSGYGTNSELSNPS | |
| | ETESERKDELSDWSLAGEDDRDSRHQRDSRRRPGGRGRSVSGG | |
| | RGRGGPRGGKSSISSVLKDPDSNPYSLLDNTESDQTADTDASES | |
| | HHSTNRRRRSRRRRTDEDAVLMDGMTESDTASVNENGLGKRC | |
| | DGSDYKDDDDK | |
| DAZ4 | MGIINTLQKYYCRVRGGRCAVLSCLPKEEQIGKCSTRGRKCCR | 65 |
| | RKKGGSGGSGGSGGSGGSGGSMAVPETRPNHTIYINNLNSKIK | |
| | KDELKKSLYAIFSQFGQILDILVPRQRTPRGQAFVIFKEVSSATN | |
| | ALRSMQGFPFYDKPMRIQYAKTDKRIPAKMKGTFVGSLQLPPL | |
| | ERLTLGGSGGSGGSGGSGGSGGSMSAANPETPNSTISREASTQS | |
| | SSAAASQGWVLPEGKIVPNTVFVGGIDARMDETEIGSCFGRYGS | |
| | VKEVKIITNRTGVSKGYGFVSFVNDVDVQKIVGSQIHFHGKKLK | |
| | LGPAIRKQKLCARHVQPRPLVVNPPPPPQFQNVWRNPNTETYL | |
| | QPQITPNPVTQHVQSAANPETPNSTISREASTQSSSAAASQGWV | |
| | LPEGKIVPNTVFVGGIDARMDETEIGSCFGRYGSVKEVKIITNRT | |
| | GVSKGYGFVSFVNDVDVQKIVGSQIHFHGKKLKLGPAIRKQKL | |
| | CARHVQPRPLVVNPPPPPQFQNVWRNPNTETYLQPQITPNPVTQ | |
| | HVQAYSAYPHSPGQVITGCQLLVYNYQEYPTYPDSAFQVTTGY | |
| | QLPVYNYQPFPAYPRSPFQVTAGYQLPVYNYQAFPAYPNSPFQ | |
| | VATGYQFPVYNYQPFPAYPSSPFQVTAGYQLPVYNYQAFPAYP | |
| | NSPFQVATGYQFPVYNYQAFPAYPNSPVQVTTGYQLPVYNYQ | |
| | AFPAYPSSPFQVTTGYQLPVYNYQAFPAYPNSAVQVTTGYQFH | |
| | VYNYQMPPQCPVGEQRRNLWTEAYKWWYLVCLIQRRDGSDY | |
| | KDDDDK* | |

### B. FIG. 8 Methods and Results

Methods: See methods described for FIG. 7.

Results: Additional dual luciferase reporter plasmids were constructed with human Jagged1 (JAG1) 3′ UTR and mouse SCN1a 3′ UTR appended to the Firefly luciferase open-reading frame. Then CIRTS-Y1 was tested for the ability to increase Firefly luciferase expression in a gRNA-dependent manner when targeting the Jag1 and SCN1a 3′ UTRs. At least one gRNA was identified by each reporter system to mediate CIRTS-Y1 and gRNA-dependent increase of Firefly expression. gRNA sequences used in FIG. 8.

| | | SEQ |
|---|---|---|
| | | ID |
| Description | Sequence | NO: |
| Jag1 g1 | GCCGGTAGAACTACGTAAGCTCAGAAGAGGCCACACTGTT | 144 |
| Jag1 g2 | GAGTATGACACGACAGTTTAAAGAACTACAAGCCCTCAGA | 145 |
| Jag1 g3 | TAAATTCTTCATTTTACCAGCAACTGCTGACATCAAAGTC | 146 |
| Jag1 g4 | TTAATCATATTCAACCACTCACATTTTGGCTATGTTAAGT | 147 |
| SCN1a g1 | CTCTCTGATTTTGAGGAAAAGAAATTCTGAAGCCCACATT | 55 |

### C. FIG. 9 Methods and Results

Methods: Neuro 2a cells were plated in a 12-well plate and transfected 24 hours later using Lipofectamine 3000 according to the manufacturer's protocol. Each well was transfected with a plasmid expressing CIRTS-Y1 and either a NT gRNA or SCN1a g1. Protein was isolated from the Neuro 2a cells 48 hours later by incubation with RIPA buffer and protease inhibitors.

For western blotting, 15 μg of total protein was loaded for each condition. SCN1a and alpha-tubulin (a-tubulin) levels were detected by incubation with anti-SCN1a, or anti-alpha-tubulin antibody followed by incubation with an appropriate secondary antibody conjugated to HRP then chemiluminescent detection.

Results: When compared to the alpha-tubulin loading control, transfection with Bdef-TBP-Y1 (SEQ ID NO:80) and SCN1a g1 appears to increase SCN1a protein present compared to the NT gRNA by approximately 1.4-fold.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. The references cited herein, to the extent that they provide exemplary procedural or other details supplementary to those set forth herein, are specifically incorporated herein by reference.

1. A polypeptide comprising an effector domain wherein the effector domain comprises the amino acid sequence of one of SEQ ID NOs: 19-39, 60-62, 69-76, 113-121, or 136-143 or comprises an amino acid sequence with at least 80% sequence identity to one of SEQ ID NOs: 19-39, 60-62, 69-76, 113-121, or 136-143; and wherein the polypeptide further comprises one or more of a stabilizer domain, linker, RNA hairpin binding domain, nuclear export signal, and molecular tag.

2. A polypeptide comprising an effector domain, wherein the effector domain comprises a fusion between at least two polypeptides, wherein each polypeptide is independently selected from one of SEQ ID NOs: 22-24, 28-39, 60-62, 69-76, 113-121, or 136-143 or a polypeptide with at least 80% sequence identity to one of SEQ ID NOs: 22-24, 28-39, 60-62, 69-76, 113-121, or 136-143.

3. The polypeptide of claim 2, wherein each polypeptide is independently selected from one of SEQ ID NOs: 32-39 or 69-76 or a polypeptide with at least 80% sequence identity to one of SEQ ID NOs: 32-39 or 69-76.

4. The polypeptide of claim 2 or 3, wherein the polypeptide comprises a linker between the two polypeptides.

5. The polypeptide of claim 4, wherein the linker comprises SEQ ID NO:15.

6. The polypeptide of any one of claims 2-5, wherein the effector domain comprises: a fusion between a polypeptide comprising at least 80% sequence identity to SEQ ID NO:32 and a polypeptide comprising at least 80% sequence identity to SEQ ID NO:33; a fusion between a polypeptide comprising at least 80% sequence identity to SEQ ID NO:34 and a polypeptide comprising at least 80% sequence identity to SEQ ID NO:33; a fusion between a polypeptide comprising at least 80% sequence identity to SEQ ID NO:34 and a polypeptide comprising at least 80% sequence identity to SEQ ID NO:32; a fusion between a polypeptide comprising at least 80% sequence identity to SEQ ID NO:35 and a polypeptide comprising at least 80% sequence identity to SEQ ID NO:36; a fusion between a polypeptide comprising at least 80% sequence identity to SEQ ID NO:36 and a polypeptide comprising at least 80% sequence identity to SEQ ID NO:37; a fusion between a polypeptide comprising at least 80% sequence identity to SEQ ID NO:38 and a polypeptide comprising at least 80% sequence identity to SEQ ID NO: 39; a fusion between a polypeptide comprising at least 80% sequence identity to SEQ ID NO: 69 and a polypeptide comprising at least 80% sequence identity to SEQ ID NO:70; or a fusion between a polypeptide comprising at least 80% sequence identity to SEQ ID NO:69 and a polypeptide comprising at least 80% sequence identity to SEQ ID NO:71.

7. The polypeptide of any one of claims 2-6, wherein the effector domain comprises: a fusion between a polypeptide comprising SEQ ID NO:32 and a polypeptide comprising SEQ ID NO: 33; a fusion between a polypeptide comprising SEQ ID NO:34 and a polypeptide comprising SEQ ID NO:33; a fusion between a polypeptide comprising SEQ ID NO:34 and a polypeptide comprising SEQ ID NO:32; a fusion between a polypeptide comprising SEQ ID NO: 35 and a polypeptide comprising SEQ ID NO:36; a fusion between a polypeptide comprising SEQ ID NO:36 and a polypeptide comprising SEQ ID NO:37; a fusion between SEQ ID NO:38 and a polypeptide comprising SEQ ID NO:39; a fusion between a polypeptide comprising SEQ ID NO:69 and a polypeptide comprising SEQ ID NO:70; or a fusion between a polypeptide comprising SEQ ID NO:69 and a polypeptide comprising SEQ ID NO:71.

8. The polypeptide of any one of claims 2-7, wherein the effector domain comprises the amino acid sequence of one of SEQ ID NOs: 19-21, 25-27, 72 or 73 or a the effector domain comprises an amino acid sequence with at least 80% sequence identity to one of SEQ ID NOs: 19-21, 25-27, 72 or 73.

9. The polypeptide of any one of claims 2-8, wherein the polypeptide further comprises one or more of a stabilizer domain, linker, RNA hairpin binding domain, nuclear export signal, and molecular tag.

10. The polypeptide of any one of claims 1-9, wherein the polypeptide comprises a RNA hairpin binding domain.

11. The polypeptide of claim 10, wherein the hairpin binding domain comprises SEQ ID NO: 16 or a polypeptide with at least 80% sequence identity to SEQ ID NO:16.

12. The polypeptide of any one of claims 1-11, wherein the polypeptide comprises a stabilizer domain.

13. The polypeptide of claim 12, wherein the stabilizer domain comprises SEQ ID NO:14, 58, or 59 or a polypeptide with at least 80% sequence identity to SEQ ID NO:14, 58, or 59.

14. The polypeptide of any one of claims 1-13, wherein the polypeptide comprises a nuclear export signal.

15. The polypeptide of claim 14, wherein the nuclear export signal comprises SEQ ID NO: 17, or a polypeptide with at least 80% sequence identity to SEQ ID NO:17.

16. The polypeptide of any one of claims 1-15, wherein the polypeptide comprises a molecular tag.

17. The polypeptide of claim 16, wherein the molecular tag comprises SEQ ID NO:18, or a polypeptide with at least 80% sequence identity to SEQ ID NO:18.

18. The polypeptide of any one of claims 1-17, wherein the hairpin binding domain is amino-proximal to the effector domain.

19. The polypeptide of any one of claims 1-18, wherein the stabilizer domain is amino-proximal to the RNA hairpin binding domain and/or effector domain.

20. The polypeptide of any one of claims 1-19, wherein the nuclear export signal is amino-proximal to the effector domain.

21. The polypeptide of any one of claims 1-20, wherein the molecular tag is carboxy-proximal to the effector domain.

22. The polypeptide of any one of claims 1-21, wherein the polypeptide comprises, from N-terminus to the C-terminus, a stabilizer domain, a glycine serine linker, a RNA hairpin binding region, a nuclear export signal, an effector domain, and a molecular tag.

23. The polypeptide of any one of claims 1-22, wherein the polypeptide comprises the amino acid sequence of one of SEQ ID NOs: 1-13, 63-68, 77-82, or 148-169 or an amino acid with at least 80% sequence identity to one of SEQ ID NOs: 1-13, 63-68, 77-82, or 148-169.

24. The polypeptide of claim 23, wherein the polypeptide comprises at least one substitution relative to SEQ ID NOs: 1-13, 63-68, 77-82, or 148-169.

25. A nucleic acid encoding the polypeptide of any one of claims 1-24.

26. A nucleic acid encoding a RNA targeting molecule comprising a RNA targeting region and at least one hairpin structure, wherein the hairpin structure of the RNA targeting molecule specifically binds to the RNA hairpin binding domain of the polypeptide.

27. The nucleic acid of claim 26, wherein the RNA targeting region hybridizes to the 5′UTR of a RNA.

28. The nucleic acid of claim 26, wherein the RNA targeting region hybridizes to the 3′UTR of a RNA.

29. The nucleic acid of claim 26 or 27, wherein the RNA targeting region hybridizes to the 5′UTR or 3′UTR of a mammalian mRNA.

30. The nucleic acid of any one of claims 26-29, wherein the RNA targeting region comprises a nucleic acid sequence of one of SEQ ID NOs: 55, 83-86, 88-112, 122-131, or 144-147 or a nucleic acid sequence having at least 80% sequence identity to one of of SEQ ID NOs: 55, 83-86, 88-112, 122-131, or 144-147.

31. The nucleic acid of any one of claims 26-30, wherein the RNA targeting molecule comprises a TAR hairpin scaffold or a human SLBP hairpin scaffold.

32. The nucleic acid of any one of claims 26-31, wherein the RNA targeting molecule comprises at least one of SEQ ID NO:40-54, 56, 57, or the sequence UCCCGA.

33. The nucleic acid of claim 32, wherein the RNA targeting molecule comprises SEQ ID NO: 40 or 41.

34. An expression vector comprising the nucleic acid of any one of claims 25-33.

35. A host cell comprising the nucleic acid of any one of claims 25-33 or expression vector of claim 34.

36. A delivery vehicle comprising the polypeptide of any one of claims 1-24, nucleic acid of any one of claims 25-33, or expression vector of claim 34.

37. The delivery vehicle of claim 36, wherein the delivery vehicle comprises liposome(s), particle(s), exosome(s), microvesicle(s), a gene-gun or one or more nucleic acid vector(s).

38. The delivery vehicle of claim 36 or 37, wherein the delivery vehicle further comprises a RNA targeting molecule comprising a RNA targeting region and at least one hairpin structure, wherein the hairpin structure of the RNA targeting molecule specifically binds to the RNA hairpin binding domain of the polypeptide.

39. The delivery vehicle of claim 38, wherein the RNA targeting region hybridizes to the 5′UTR of a RNA.

40. The delivery vehicle of claim 38, wherein the RNA targeting region hybridizes to the 3′UTR of a RNA.

41. The delivery vehicle of claim 39 or 40, wherein the RNA targeting region hybridizes to the 5′UTR or 3′UTR of a mammalian mRNA.

42. The delivery vehicle of any one of claims 38-41, wherein the RNA targeting region comprises a nucleic acid sequence of one of SEQ ID NOs: 55, 83-86, 88-112, 122-131, or 144-147 or a nucleic acid sequence having at least 80% sequence identity to one of SEQ ID NOs: 55, 83-86, 88-112, 122-131, or 144-147.

43. The delivery vehicle of any one of claims 38-42, wherein the RNA targeting molecule comprises a TAR hairpin scaffold or a human SLBP hairpin scaffold.

44. The delivery vehicle of any one of claims 38-43, wherein the RNA targeting molecule comprises at least one of SEQ ID NO:40-54, 56, 57, or the sequence UCCCGA.

45. The delivery vehicle of claim 44, wherein the RNA targeting molecule comprises SEQ ID NO: 40 or 41.

46. A composition comprising the polypeptide of any one of claims 1-24, nucleic acid of any one of claims 25-33, expression vector of claim 34, host cell of claim 35, or delivery vehicle of any one of claims 36-45.

47. The composition of claim 46, wherein the composition further comprises a RNA targeting molecule comprising a RNA targeting region and at least one hairpin structure, wherein the hairpin structure of the RNA targeting molecule specifically binds to the RNA hairpin binding domain of the polypeptide.

48. The composition of claim 47, wherein the RNA targeting region hybridizes to the 5′UTR of a RNA.

49. The composition of claim 47, wherein the RNA targeting region hybridizes to the 3′UTR of a RNA.

50. The composition of claim 48 or 49, wherein the RNA targeting region hybridizes to the 5′UTR or 3′UTR of a mammalian mRNA.

51. The composition of any one of claims 47-50, wherein the RNA targeting region comprises a nucleic acid sequence of one of SEQ ID NOs: 55, 83-86, 88-112, 122-131, or 144-147 or a nucleic acid sequence having at least 80% sequence identity to one of SEQ ID NOs: 55, 83-86, 88-112, 122-131, or 144-147.

52. The composition of any one of claims 47-51, wherein the RNA targeting molecule comprises a TAR hairpin scaffold or a human SLBP hairpin scaffold.

53. The composition of any one of claims 47-52, wherein the RNA targeting molecule comprises at least one of SEQ ID NO:40-54, 56, 57, or the sequence UCCCGA.

54. The composition of claim 53, wherein the RNA targeting molecule comprises SEQ ID NO: 40 or 41.

55. A method of making a polypeptide comprising transferring the nucleic acid of claim 25 or expression vector of claim 34 into a cell and isolating the expressed polypeptide.

56. A method of modulating at least one target RNA comprising contacting the target RNA with the polypeptide of any one of claims 1-24, the delivery vehicle of any one of claims 36-45, or the composition of any one of claims 46-54.

57. A method for modulating at least one target RNA in a subject, the method comprising administering the polypeptide of any one of claims 1-24, the delivery vehicle of any one of claims 36-45, or the composition of any one of claims 46-54 to the subject.

58. The method of claim 56 or 57, wherein modulating the at least one target RNA comprises cleaving, demethylating, methylating, activating translation, repressing translation, promoting degradation, and/or binding to the RNA.

59. The method of claim 56 or 58, wherein the target RNA is in a prokaryotic or eukaryotic cell.

60. The method of claim 59, wherein the target RNA is in a human cell.

61. The method of claim 59 or 60, wherein the target RNA is in vitro or in vivo.

62. A cell or progeny thereof comprising modulated target RNA, wherein the target RNA has been modulated according to any one of claims 56-61.

63. A multicellular organism comprising one or more cells according to claim 62.

64. A plant or animal comprising one or more cells according to claim 63.

65. A kit comprising the polypeptide of any one of claims 1-24, nucleic acid of any one of claims 25-33, expression vector of claim 34, host cell of claim 35, delivery vehicle of any one of claims 36-45, or the composition of any one of claims 46-54.
